(19) United States
(12) Patent Application Publication
(10) Pub. No.: US 2025/0206784 A1
(43) Pub. Date: Jun. 26, 2025

(54) MODIFIED SPIKE PROTEINS AND USES THEREOF

(71) Applicants: CILOA, Montpellier (FR); JERID, Saint-André-de-Sangonis (FR)

(72) Inventors: Zaïne El Abiddine Robert MAMOUN, Saint-André-de-Sangonis (FR); Bernadette Nadine TRENTIN, Montpellier (FR); Katarzyna POLAK, Montpellier (FR); Maëlle LACHAT, Montpellier (FR)

(73) Assignees: CILOA, Montpellier (FR); JERID, Saint-André-de-Sangonis (FR)

(21) Appl. No.: 18/851,843

(22) PCT Filed: Mar. 29, 2023
(86) PCT No.: PCT/EP2023/058193

§ 371 Date: Sep. 27, 2024

(30) Foreign Application Priority Data
Mar. 29, 2022 (EP) 22305397.6

(51) Int. Cl. C07K 14/005; A61K 39/00; A61K 39/215; A61P 31/14; G01N 33/569

(52) U.S. Cl. CPC C07K 14/005; A61K 39/215; A61P 31/14; G01N 33/56983; A61K 2039/60; C12N 2770/20022; C12N 2770/20034; G01N 2333/165; G01N 2469/20

(57) ABSTRACT

A nucleic acid coding for a modified Spike protein from a virus of the Orthocoronavirinae subfamily, an extracellular vesicle expressing a modified Spike protein from a virus of the Orthocoronavirinae subfamily, and a population of the extracellular vesicles. Also, the use of the nucleic acid, extracellular vesicle or population of extracellular vesicles for use in a method of immunizing a subject against a virus of the Orthocoronavirinae subfamily, in methods of production and screening of neutralizing antibodies.

### FIELD OF INVENTION

The present invention relates to a nucleic acid coding for a modified Spike protein from a virus of the Orthocoronavirinae subfamily, an extracellular vesicle expressing a modified Spike protein from a virus of the Orthocoronavirinae subfamily, and a population of said extracellular vesicles. The present invention further relates to the use of said nucleic acid, extracellular vesicle or population of extracellular vesicles, for use in a method of immunizing a subject against a virus of the Orthocoronavirinae subfamily, in methods of production and screening of neutralizing antibodies.

### BACKGROUND OF INVENTION

Orthocoronavirinae, more commonly known as coronaviruses, are enveloped, positive-sense single-stranded RNA viruses which infect birds and mammals.

Coronaviruses are divided into four genera: alphacoronavirus, betacoronavirus, gammacoronavirus and deltacoronavirus. Alphacoronaviruses and betacoronaviruses infect mammals, while gammacoronaviruses and deltacoronaviruses primarily infect birds. Among the betacoronaviruses, the most pathogenic coronaviruses for humans include Severe Acute Respiratory Syndrome Coronavirus 1 (SARS-CoV-1), the Middle Eastern respiratory syndrome coronavirus (MERS-CoV), and now Severe Acute Respiratory Syndrome Coronavirus 2 (SARS-CoV-2).

Vaccines have been developed to limit further spread of infection of coronaviruses. Most of those vaccines are designed to recognize proteins from specific strains of coronaviruses. However, the coronaviruses often mutated and the new generated variants may therefore escape from the existing vaccines. In the case of SARS-CoV-2 for example, the emergence of the Omicron (B.1.1.529) variant has generated serious concern about the continued efficacy of vaccines that were designed from the original strain of SARS-CoV-2 detected in Wuhan (China) in 2019. Thus, there is an urgent need to develop broadly protective vaccines that cover whole virus families or genera.

In the present application, the Applicant has developed a unique vaccine strategy, based inter alia on chimeric Spike proteins, that induces an immune response against several coronavirus strains at once.

### SUMMARY

The present invention relates to a nucleic acid comprising:
(i) a sequence coding for the S1 domain of a Spike protein from a virus of the Orthocoronavirinae subfamily, or a variant thereof;
(ii) a sequence coding for the S2 domain of a Spike protein from a virus of the Orthocoronavirinae subfamily, or a variant thereof; and
(iii) a sequence coding for a pilot peptide which interacts with ESCRT proteins,
wherein the sequences (i) and (ii) are from Spike proteins from a different virus of the Orthocoronavirinae subfamily, or from Spike proteins from different strains of the same virus of the Orthocoronavirinae subfamily.

In one embodiment, the sequence (ii) is from a Spike protein from Severe Acute Respiratory Syndrome Coronavirus 2 (SARS-CoV-2).

In one embodiment, the sequence (i) is from a Spike protein from a virus of the Orthocoronavirinae subfamily selected from the group comprising or consisting of Middle East respiratory syndrome-related coronavirus (MERS), Severe Acute Respiratory Syndrome Coronavirus 1 (SARS-CoV-1) and SARS-CoV-2.

In one embodiment, the sequence (ii) is from a Spike protein from SARS-CoV-2 Alpha strain or a sublineage thereof, and the sequence (i) is from a Spike protein from a virus of the Orthocoronavirinae subfamily selected from the group comprising or consisting of MERS, SARS-CoV-1 and the SARS-CoV2 Beta, Gamma, Delta and Omicron strains or sublineages thereof.

In one embodiment, the S2 domain of a Spike protein is modified to comprise at least one proline residue, preferably two consecutive proline residues, between the amino acid motifs K and V in the amino acid motif RLDKV (SEQ ID NO: 39).

In one embodiment, the pilot peptide comprises at least one YxxL motif or DYxxL motif, and at least one PxxP motif, in which “x” represents any amino acid residue,

In one embodiment, the pilot peptide comprises an amino acid sequence with SEQ ID NO: 8 or a variant thereof, with the proviso that a variant of SEQ ID NO: 8 retains three YxxL motifs and four PxxP motifs, in which “x” represents any amino acid residue.

The present invention also relates to the nucleic acid of the invention, being inserted into a nucleic acid expression vector, and being operably linked to regulatory elements.

The present invention also relates to an extracellular vesicle, preferably an exosome, harboring at its external surface a Spike protein from a virus of the Orthocoronavirinae subfamily, or a variant thereof, wherein said Spike protein comprises:
(i) a sequence of the S1 domain of a Spike protein from a virus of the Orthocoronavirinae subfamily, or a variant thereof;
(ii) a sequence of the S2 domain of a Spike protein from a virus of the Orthocoronavirinae subfamily, or a variant thereof; and
wherein the sequences (i) and (ii) are from Spike proteins from a different virus of the Orthocoronavirinae subfamily, or from Spike proteins from different strains of the same virus of the Orthocoronavirinae subfamily.

In one embodiment, the sequence (ii) is from a Spike protein from SARS-CoV-2.

In one embodiment, the sequence (i) is from a Spike protein from a virus of the Orthocoronavirinae subfamily selected from the group comprising or consisting of MERS, SARS-CoV-1 and SARS-CoV-2.

In one embodiment, the extracellular vesicle of the invention is obtainable by a method comprising steps of:
1) transfecting cells with the nucleic acid of the invention;
2) culturing the transfected cells for a time sufficient to allow extracellular vesicle production; and
3) purifying said extracellular vesicle.

The present invention also relates to a population of extracellular vesicles of the invention.

The present invention also relates to the nucleic acid of the invention, for use in a method of immunizing a subject against a virus of the Orthocoronavirinae subfamily, said method comprising:
1) at least one priming step, wherein at least one nucleic acid of the invention is to be administered to said subject; and
2) optionally at least one boosting step, wherein the extracellular vesicle of the invention or the population of extracellular vesicles of the invention, or an extracellular vesicle harboring at its external surface a Spike protein from a virus of the Orthocoronavirinae subfamily or a variant thereof or a population of said extracellular vesicles is to be administered to said subject,
thereby immunizing the subject against a virus of the Orthocoronavirinae subfamily, preferably wherein said method comprises two iterations of the priming step and one iteration of the boosting step.

In one embodiment, the Spike protein harbored at the extracellular vesicle surface comprises S1 and S2 domains derived from the same Spike protein. In one embodiment, the method comprises steps 1) and 2).

In one embodiment, the method of immunizing a subject further comprises at least one step of administering a nucleic acid comprising a sequence of a S gene coding for a Spike protein from a virus of the Orthocoronavirinae subfamily, or a variant thereof, preferably wherein said method further comprises three iterations of said step.

The present invention also relates to the nucleic acid of the invention, for use in a method of producing neutralizing antibodies, said method comprising at least one step of administering at least one nucleic acid of the invention.

The present invention also relates to a nucleic acid, an extracellular vesicle; or a population of extracellular vesicles as defined hereinabove, for use in an in vitro method of producing neutralizing antibodies.

The present invention also relates to a nucleic acid, an extracellular vesicle; or a population of extracellular vesicles, for use in an in vivo method of producing neutralizing antibodies, thereby preventing or treating an Orthocoronavirinae infectious disease in a subject in need thereof, said method comprising at least one step of administering at least one nucleic acid, an extracellular vesicle, or a population of extracellular vesicles as defined hereinabove to a subject.

The present invention further relates to a method of screening antibodies against the S2 domain of a Spike protein of a virus of the Orthocoronavirinae subfamily comprising the steps of:
(a) contacting the antibodies to be screened with a first extracellular vesicle, or a first population of extracellular vesicles harboring a Spike protein at the extracellular vesicle surface,
(b) selecting the antibodies which bind to the Spike protein harbored at the extracellular vesicle surface,
(c) contacting the antibodies selected in step (b) with a second extracellular vesicle or a second population of extracellular vesicles, wherein the extracellular vesicle(s) used in step (c) harbor(s) a Spike protein comprising the same S2 domain but a different S1 domain than the Spike protein harbored at the surface of the extracellular vesicle(s) used in step (a),
(d) selecting the antibodies which bind to the Spike protein harbored at the extracellular vesicle surface, thereby identifying antibodies which bind specifically to the S2 domain of Spike protein.

### Definitions

“Adjuvant” refers to a molecule or complex of molecules which allow(s) or otherwise facilitate(s) (1) the mobilization of antigen-presenting and/or polymorphonuclear cells; (2) the antigen uptake and presentation of the antigen(s) in a vaccine by antigen-presenting cells; (3) the secretion of proteins by antigen-presenting cells; (4) the recruitment, targeting and activation of antigen-specific cells; (5) the modulation of activities that regulate the ensuing immune responses; and/or (6) the protection of the antigen from degradation and elimination.

“Derived from” refers to a nucleic acid or amino acid sequence that may be obtained from a particular specified nucleic acid or amino acid sequence. This term includes, for example, any sequence obtained from a native nucleic acid sequence coding for a Spike protein, or the corresponding native amino acid sequence, and which is modified by insertions, deletions, or substitutions of nucleotides or amino acid residues.

“ESCRT” or “endosomal sorting complexes required for transport” refers originally to a cellular machinery made up of five multi-subunit protein complexes, which act cooperatively at specialized endosomes to facilitate the movement of specific cargoes from the limiting membrane into vesicles that bud into the endosome lumen. This machinery is hijacked by several envelope viruses to bud from cellular membranes, including the plasma membrane.

“Exosome” refers to an extracellular vesicle that is produced in the endosomal compartment of eukaryotic cells (Théry et al., 2018. J Extracell Vesicles. 7(1):1535750; Yáñez-Mó et al., 2015. J Extracell Vesicles. 4:27066; van Niel et al., 2018. Nat Rev Mol Cell Biol. 19(4):213-228). Typically, exosomes harbor at their surface the CD81, CD63 and CD9 markers.

“Expression vector” refers to a vector capable of directing expression of a nucleic acid sequence of interest (such as, e.g., a nucleic acid according to the present invention) in an appropriate host cell, comprising a promoter operatively linked to the nucleic acid sequence of interest, itself operatively linked to a termination sequence.

“Extracellular vesicle” refers to any vesicle composed of a lipid bilayer that are naturally released from a cell and comprise a cytosolic fraction of said cell. This expression in particular includes vesicles secreted into the extracellular space, i.e., “exosomes”.

“Global alignment” refers to alignment that aligns two sequences from beginning to end, aligning each letter in each sequence only once. An alignment is produced, regardless of whether or not there is similarity or identity between the sequences. For example, 50% sequence identity based on global alignment means that in an alignment of the full sequence of two compared sequences, each of 100 nucleotides or amino acid residues in length, 50% of the residues are the same. It is understood that global alignment can also be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman & Wunsch, 1970. J Mol Biol. 48(3):443-53). Exemplary programs and software for performing global alignment are publicly available and include the Global Sequence Alignment Tool available at the National Center for Biotechnology Information (NCBI) website (http://ncbi.nlm.nih.gov), and the program available at deepc2.psi.iastate.edu/aat/align/align.html.

“Identity” or “sequence identity”: refers to the number of identical or similar nucleotides or amino acid residues in a comparison between a test and a reference sequence. Sequence identity can be determined by sequence alignment of nucleic acid or amino acid sequences to identify regions of similarity or identity. For purposes herein, sequence identity is generally determined by alignment to identify identical nucleotides or amino acid residues. The alignment can be local or global. Matches, mismatches and gaps can be identified between compared sequences. Gaps are null nucleotides or amino acid residues inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps as

$$\frac{number of identical positions}{length of the total aligned sequence} \times 100.$$

For purposes herein, sequence identity can be determined by standard alignment algorithm programs used with default gap penalties established by each supplier. Default parameters for the GAP program can include:
a unary comparison matrix (containing a value of 1 for identities and 0 for non-identities) and the weighted comparison matrix of Gribskov & Burgess (1986. Nucleic Acids Res. 14(16):6745-63), as described by Schwartz & Dayhoff (1979. Matrices for detecting distant relationships. In Dayhoff (Ed.), Atlas of protein sequences. 5:353-358. Washington, DC: National Biomedical Research Foundation);
a penalty of 3.0 for each gap and an additional 0.10 penalty for each symbol in each gap; and
no penalty for end gaps.

“Isolated” and any declensions thereof, as well as “purified” and any declensions thereof, are used interchangeably, and mean that a molecular entity to which it refers (e.g., a protein or peptide, a nucleic acid, an extracellular vesicle, etc.) is substantially free of other components (i.e., of contaminants) found in the natural environment in which said molecular entity is normally found. Preferably, an isolated or purified molecular entity (e.g., an isolated or purified protein or peptide, an isolated or purified nucleic acid, an isolated or purified extracellular vesicle, etc.) is substantially free of other molecular entities with which it is associated in a cell or a virus. By “substantially free”, it is meant that said isolated or purified molecular entity represents more than 50% of a heterogeneous composition (i.e., is at least 50% pure), preferably, more than 60%, more than 70%, more than 80%, more than 90%, more than 95%, and more preferably still more than 98% or 99%. Purity can be evaluated by various methods known by the one skilled in the art, including, but not limited to, chromatography, gel electrophoresis, immunoassay, composition analysis, biological assay, and the like.

“Kit-of-parts” refers to a kit comprising a plurality of (different) items that may be functionally used together, concomitantly or one after another.

“Local alignment” refers to an alignment that aligns two sequence, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Smith & Waterman, 1981. Adv Appl Math. 2(4):482-9). For example, 50% sequence identity based on local alignment means that in an alignment of the full sequence of two compared sequences of any length, a region of similarity or identity of 100 nucleotides or amino acid residues in length has 50% of the residues that are the same in the region of similarity or identity.

“Native” refers to a nucleic acid sequence or an amino acid sequence that is normally expressed and present in a wild-type microorganism, e.g. a virus, in nature.

“Orthocoronavirinae infectious disease” refers to a disease caused by infection with a virus of the Orthocoronavirinae subfamily.

“Orthocoronavirinae subfamily” refers to one of the two sub-families in the family Coronaviridae, order Nidovirales, and realm Riboviria. The members of the Orthocoronavirinae subfamily are known as coronaviruses. These are enveloped viruses with a positive-sense single-stranded RNA genome and a nucleocapsid of helical symmetry. The genome size of coronaviruses ranges from approximately 26 to 32 kilobases, one of the largest among RNA viruses.

“Regulatory element” refers to nucleic acid sequences which are necessary or desirable to affect the expression of coding and non-coding sequences of interest (such as, e.g., a nucleic acid according to the present invention), to which they are operatively linked. Examples of regulatory elements include, but are not limited to, initiation signals, enhancers, regulators, promoters, and termination sequences. The nature and use of such regulatory sequences can differ, as is well known to the one skilled in the art, depending upon the host organism or cell.

“S gene” refers to a gene found in viruses of the Orthocoronavirinae subfamily, coding for the Spike protein.

“Spike” or “Spike protein”, also known as “surface glycoprotein”, refers to a transmembrane protein, also termed peplomer, found in viruses of the Orthocoronavirinae subfamily, and protruding from the virus surface as spikes, hence its name.

“Sublineage” refers to a group of similar viruses within a lineage. As used herein, a lineage refers to a group of genetically similar viruses with a common ancestor.

“Variant”: refers to nucleic acid or amino acid sequences that typically differs from a nucleic acid or an amino acid sequence specifically disclosed herein in one or more substitutions, deletions, additions and/or insertions. When referring to percentage of identity, it is meant a nucleic acid or amino acid sequence comprising at least 70%, preferably at least 75%, 80%, 85%, 90%, 95%, 96%, 97%, 98%, 99% or more sequence identity, preferably local sequence identity with the reference nucleic acid or amino acid sequence. In one embodiment, when referring to a variant of S1 or S2 domain of a Spike protein from a virus of the Orthocoronavirinae subfamily, said variant is from (or derived from) a virus of the Orthocoronavirinae subfamily, whose entire genome has at least 70%, preferably at least 75%, 80%, 85%, 90%, 95%, 96%, 97%, 98%, 99% or more sequence identity with the entire genome of the reference virus. In one embodiment, when referring to a variant of S1 or S2 domain of a Spike protein from a virus of the Orthocoronavirinae subfamily, said variant is from (or derived from) a virus of the Orthocoronavirinae subfamily, whose entire genome has at least 70%, preferably at least 75%, 80%, 85%, 90%, 95%, 96%, 97%, 98%, 99% or more sequence identity with the entire genome of the reference virus and said variant has a sequence comprising at least 70%, preferably at least 75%, 80%, 85%, 90%, 95%, 96%, 97%, 98%, 99% or more sequence identity, preferably local sequence identity with the reference nucleic acid or amino acid sequence.

“Vector” refers to a nucleic acid capable of transporting a nucleic acid of interest (such as, e.g., a nucleic acid according to the present invention) to which it has been linked. Vectors capable of directing the expression of a nucleic acid of interest (such as, e.g., a nucleic acid according to the present invention) are referred to as “expression vectors”. In general, expression vectors are in the form of plasmids. Herein, the terms “plasmid” and “vector” are used interchangeably. However, other forms of expression vectors, which serve equivalent functions, are also encompassed under the term vector.

### DETAILED DESCRIPTION

The present invention relates to a nucleic acid comprising:
(i) a sequence coding for a S1 domain of a Spike protein from a virus of the Orthocoronavirinae subfamily, or a variant thereof;
(ii) a sequence coding for a S2 domain of a Spike protein from a virus of the Orthocoronavirinae subfamily, or a variant thereof; and
(iii) a sequence coding for a pilot peptide which interacts with ESCRT proteins,
wherein the sequences (i) and (ii) are from (or derived from) Spike proteins from different viruses of the Orthocoronavirinae subfamily, or from Spike proteins from different strains of the same virus of the Orthocoronavirinae subfamily.

As mentioned hereinabove, the Orthocoronavirinae, more commonly known as coronaviruses, are divided into four genera: alphacoronavirus, betacoronavirus, gammacoronavirus and deltacoronavirus.

Exemplary species of alphacoronaviruses include, but are not limited to, alphacoronavirus 1 (also known as transmissible gastroenteritis virus, transmissible gastroenteritis coronavirus, or TGEV), human coronavirus 229E (also known as HCoV-229E), human coronavirus NL63 (also known as HCoV-NL63), miniopterus bat coronavirus 1 (also known as Bat-CoV MOP1), miniopterus bat coronavirus HKU8 (also known as Bat-CoV HKU8), porcine epidemic diarrhea virus (also known as PED virus or PEDV), rhinolophus bat coronavirus HKU2 (also known as Chinese horseshoe bat virus or Bat-CoV HKU2), and scotophilus bat coronavirus 512 (also known as Bat-CoV 512).

Exemplary species of betacoronaviruses include, but are not limited to, betacoronavirus 1 (including, without limitation, bovine coronavirus and human coronavirus OC43), hedgehog coronavirus 1, human coronavirus HKU1 (also known as HCoV-HKU1), Middle East respiratory syndrome-related coronavirus (also known as MERS-CoV, EMC/2012 or HCoV-EMC/2012), murine coronavirus (also known as M-CoV), pipistrellus bat coronavirus HKU5 (also known as Bat-CoV HKU5), rousettus bat coronavirus HKU9 (also known as HKU9-1), severe acute respiratory syndrome-related coronavirus (also known as SARSr-CoV or SARS-CoV; and including, without limitation, SARS-CoV and SARS-CoV-2), and tylonycteris bat coronavirus HKU4 (also known as Bat-CoV HKU4).

Exemplary species of gammacoronaviruses include, but are not limited to, avian coronavirus (also known as IBV) and beluga whale coronavirus SW1 (also known as Whale-CoV SW1).

Exemplary species of deltacoronaviruses include, but are not limited to, bulbul coronavirus HKU11 (also known as Bulbul-CoV HKU11) and porcine coronavirus HKU15 (also known as porcine coronavirus HKU15 or PorCoV HKU15).

In one embodiment, the virus of the Orthocoronavirinae subfamily is a virus of the betacoronavirus genus.

In one embodiment, the sequence (i) is from (or is derived from) a Spike protein from a virus of the betacoronavirus genus. In one embodiment, the sequence (i) is from (or is derived from) a Spike protein from respiratory syndrome-related coronaviruses.

In one embodiment, the sequence (i) is from (or is derived from) a Spike protein from a virus of the Orthocoronavirinae subfamily selected from the group comprising or consisting of Middle East respiratory syndrome-related coronavirus (MERS), Severe Acute Respiratory Syndrome Coronavirus 1 (SARS-CoV-1) and SARS-CoV-2.

In one embodiment, the sequence (i) is from (or is derived from) a Spike protein from a virus of the Orthocoronavirinae subfamily selected from the group comprising or consisting of MERS, SARS-CoV-1 and the SARS-CoV-2 Alpha, Beta, Gamma, Delta, Omicron, Lambda, Mu, Kappa, Iota, Eta, Epsilon, Zeta and Theta strains or sublineages thereof.

In one embodiment, the sequence (i) is from (or is derived from) a Spike protein from a virus of the Orthocoronavirinae subfamily selected from the group comprising or consisting of MERS, SARS-CoV-1 and the SARS-CoV-2 Alpha, Beta, Gamma, Delta and Omicron strains or sublineages thereof.

In one embodiment, the sequence (i) is from (or is derived from) a Spike protein from a virus of the Orthocoronavirinae subfamily selected from the group comprising or consisting of MERS, SARS-CoV-1 and the SARS-CoV-2 Beta, Gamma, Delta and Omicron strains or sublineages thereof.

As used herein, strains of SARS-CoV-2 are named according to the nomenclature established by the WHO label, i.e. use of Greek alphabet. However, the one skilled in the art knows the correspondence between the WHO labels of coronaviruses and other nomenclatures, and thus will not have any difficulty to understand which strains are disclosed herein.

Examples of nomenclature of SARS-CoV-2 strains are provided hereinbelow:
Original Wuhan L strain and its sub-variants, some examples being S, V, G, GH, GR strains,
SAlpha strain, corresponding to SARS-CoV-2 UK strain #B.1.1.7,
Beta strain, corresponding to SARS-CoV-2 South African strain #B.1.351,
Gamma strain, corresponding to SARS-CoV-2 Brazilian strain #P.1,
Delta strain, corresponding to SARS-CoV-2 Indian strain #B.1.617,
Omicron strain, corresponding to SARS-CoV-2 strain #B.1.1.529,
Lambda strain, corresponding to SARS-CoV-2 Peruvian strain #C.37,
Mu strain, corresponding to SARS-CoV-2 Colombian strain #B.1.621,
Kappa strain, corresponding to SARS-CoV-2 strain #B.1.617.1,
Iota strain, corresponding to SARS-CoV-2 strain #B.1.526,
Eta strain, corresponding to SARS-CoV-2 strain #B.1.525,
Epsilon strain, corresponding to SARS-CoV-2 strain #B.1.427/B.1.429,
Zeta strain, corresponding to SARS-CoV-2 strain #P.2,
Theta strain, corresponding to SARS-CoV-2 strain #P.3.

In one embodiment, the sequence (i) is from (or is derived from) a Spike protein from a virus of the Orthocoronavirinae subfamily selected from the group comprising or consisting of MERS, SARS-CoV-1 and the SARS-CoV-2 Omicron strain or a sublineage thereof. In one embodiment, the sequence (i) is from (or is derived from) a Spike protein from MERS or SARS-CoV-1.

In one embodiment, the sequence (i) codes for a S1 domain, wherein the entirety of the S1 domain is from (or is derived from) the same Spike protein.

In one embodiment, the sequence (i) codes for a S1 domain comprising two parts, wherein the C-terminal part (herein referred as the S1-C terminus) and the N-terminal part are from (or are derived from) different Spike proteins.

In one embodiment, said S1-C-terminus comprises between 10 and 20 amino acid residues, preferably between 12 and 15 amino acid residues, more preferably comprises 13 amino acid residues. In one embodiment, said S1-C-terminus starts with a SYQ motif and ends with a RRAR motif (SEQ ID NO: 10).

In one embodiment, the sequence (i) codes for a S1 domain comprising two parts, wherein the C-terminal part (herein referred as the S1-C terminus) and the N-terminal part are from (or are derived from) different Spike proteins, wherein the S1-C terminus is from the same Spike protein from which the S2 domain is derived.

In one embodiment, the sequence (i) codes for a S1 domain comprising two parts, wherein the S1-C terminus is the S1-C terminus of a Spike protein from SARS-CoV-2, preferably from SARS-CoV-2 Alpha strain or a sublineage thereof.

In one embodiment, the sequence coding for the S1-C terminus of a Spike protein from SARS-CoV-2 Alpha strain is the sequence SEQ ID NO: 37, corresponding to the amino acid sequence with SEQ ID NO: 38.

Thus, in one embodiment, the sequence (i) codes for a S1 domain comprising two parts, wherein the N-terminal part is from (or is derived from) a Spike protein from a virus of the Orthocoronavirinae subfamily, and wherein the S1-C terminus is coded by the sequence SEQ ID NO: 37 or a variant thereof sharing at least 70%, 75%, 80%, 85%, 90%, 95%, 96%, 97%, 98%, 99% or more sequence identity with said sequence.

In one embodiment, the sequence (i) codes for a S1 domain comprising two parts, wherein the N-terminal part is from (or is derived from) a Spike protein from a virus of the Orthocoronavirinae subfamily, and wherein the S1-C terminus is the amino acid sequence SEQ ID NO: 38 or a variant thereof sharing at least 70%, 75%, 80%, 85%, 90%, 95%, 96%, 97%, 98%, 99% or more sequence identity with said sequence.

In one embodiment, the sequence (i) further comprises a sequence coding for a signal peptide.

In one embodiment, said signal peptide is from the same Spike protein from which the S1 domain (or the N-terminal part of the S1 domain) is derived (i.e. native signal peptide).

Examples of signal peptides include, for example, the signal peptide from the Spike protein of SARS-CoV-1 strain Tor2 with sequence SEQ ID NO: 82, coded by the nucleic acid sequence SEQ ID NO: 83; the signal peptide from the Spike protein of MERS with sequence SEQ ID NO: 84, coded by the nucleic acid sequence SEQ ID NO: 85; and the signal peptide from the Spike protein of SARS-CoV-2 strain Omicron with sequence SEQ ID NO: 86, coded by the nucleic acid sequence SEQ ID NO: 87.

In one embodiment, the signal peptide is not the native signal peptide. Examples of signal peptides include, but are not limited to, the signal peptide of the metabotropic glutamate receptor 5 (mGluR5 or GlutR5).

In one embodiment, the sequence coding for the signal peptide of mGluR5 is SEQ ID NO: 5, corresponding to the amino acid sequence SEQ ID NO: 6.

Thus, in one embodiment, the sequence (i) further comprises a sequence coding for a signal peptide, said sequence coding for a signal peptide being SEQ ID NO: 5 or a variant thereof sharing at least 70%, 75%, 80%, 85%, 90%, 95%, 96%, 97%, 98%, 99% or more sequence identity with said sequence.

In one embodiment, the sequence (i) further comprises a sequence coding for a signal peptide having the amino acid sequence SEQ ID NO: 6 or a variant thereof sharing at least 70%, 75%, 80%, 85%, 90%, 95%, 96%, 97%, 98%, 99% or more sequence identity with said sequence.

In one embodiment, the sequence (i) codes for the N-terminal part of the S1 domain of a Spike protein from MERS, and a S1-C terminus of a Spike protein from SARS-CoV-2, preferably SARS-CoV-2 Alpha strain or a sublineage thereof.

In one embodiment, said sequence (i) comprises or consists of SEQ ID NO: 42, corresponding to the amino acid sequence with SEQ ID NO: 43 or a variant thereof sharing at least 70%, 75%, 80%, 85%, 90%, 95%, 96%, 97%, 98%, 99% or more sequence identity with said sequences.

In one embodiment, the sequence (i) codes for the N-terminal part of the S1 domain of a Spike protein from SARS-CoV-1, preferentially SARS-CoV-1 strain Tor2, and a S1-C terminus of a Spike protein from SARS-CoV-2, preferably SARS-CoV-2 Alpha strain or a sublineage thereof.

In one embodiment, said sequence (i) comprises or consists of SEQ ID NO: 44, corresponding to the amino acid sequence with SEQ ID NO: 45 or a variant thereof sharing at least 70%, 75%, 80%, 85%, 90%, 95%, 96%, 97%, 98%, 99% or more sequence identity with said sequences.

In one embodiment, the sequence (i) codes for the N-terminal part of the S1 domain of a Spike protein from SARS-CoV-2, preferentially SARS-CoV-2 Omicron strain, and a S1-C terminus of a Spike protein from SARS-CoV-2, preferably SARS-CoV-2 Alpha strain or a sublineage thereof.

In one embodiment, said sequence comprises or consists of SEQ ID NO: 46, corresponding to the amino acid sequence with SEQ ID NO: 47, or a variant thereof sharing at least 70%, 75%, 80%, 85%, 90%, 95%, 96%, 97%, 98%, 99% or more sequence identity with said sequences.

In one embodiment, the sequence (ii) is from (or derived from) a Spike protein from a virus of the betacoronavirus genus. In one embodiment, the sequence (ii) is from (or derived from) a Spike protein from respiratory syndrome-related coronaviruses.

In one embodiment, the sequence (ii) is from (or derived from) a Spike protein from a virus of the Orthocoronavirinae subfamily selected from the group comprising or consisting of MERS, SARS-CoV-1 and SARS-CoV-2.

In one embodiment, the sequence (ii) is from (or derived from) a Spike protein from SARS-CoV-2. In one embodiment, the sequence (ii) is from (or derived from) a Spike protein from a SARS-CoV-2 strain selected from the group comprising or consisting of Alpha, Beta, Gamma, Delta and Omicron strains, more preferably SARS-CoV-2 Alpha strain or a sublineage thereof.

In one embodiment, the sequence (ii) codes for a modified S2 domain of a Spike protein from a virus of the Orthocoronavirinae subfamily selected from the group comprising or consisting of MERS, SARS-CoV-1 and SARS-CoV-2.

In one embodiment, the sequence (ii) codes for a modified S2 domain of a Spike protein from SARS-CoV-2, preferably SARS-CoV-2 Alpha strain or a sublineage thereof.

In one embodiment, the modified S2 domain comprises the substitution of the Lysine (K) and Valine (V) residues in the amino acid motif RLDKV (SEQ ID NO: 39) by two proline (P) residues.

In one embodiment, the modified S2 domain comprises the insertion of at least one proline (P) residue between the Lysine (K) and Valine (V) residues in the amino acid motif RLDKV (SEQ ID NO: 39). In one embodiment, the modified S2 domain comprises the insertion of two proline (P) residues between the Lysine (K) and Valine (V) residues in the amino acid motif RLDKV (SEQ ID NO: 39).

In one embodiment, the insertion of 2 Prolines (PP) between the Lysine (K) and Valine (V) residues does not change the shape of the spike trimer while blocking the helices in a hairpin pre-fusion conformation.

In one embodiment, the sequence (ii) codes for a S2 domain of a Spike protein from SARS-CoV-2, preferably SARS-CoV-2 Alpha strain or a sublineage thereof, wherein said S2 domain is modified by the insertion of at least one proline (P) residue, preferably two consecutive proline residues, between the Lysine (K) and Valine (V) residues in the amino acid motif RLDKV (SEQ ID NO: 39).

In one embodiment, the totality or part of the cytosolic domain of the S2 domain of a Spike protein from SARS-CoV-2, preferably SARS-CoV-2 Alpha strain or a sublineage thereof, is removed.

In one embodiment, the modified S2 domain of a Spike protein from SARS-CoV-2, preferably SARS-CoV-2 Alpha strain or a sublineage thereof, does not comprise the cytosolic domain.

In one embodiment, the cytosolic domain of a Spike protein from SARS-CoV-2 consists of the amino acid sequence SEQ ID NO: 88.

SEQ ID NO: 88
MTSCCSCLKGCCSCGSCCKFDEDDSEPVLKGVKLHYT

In one embodiment, the totality or part of the transmembrane domain of the S2 domain of a Spike protein from SARS-CoV-2, preferably SARS-CoV-2 Alpha strain or a sublineage thereof, is removed.

In one embodiment, the modified S2 domain of a Spike protein from SARS-CoV-2, preferably SARS-CoV-2 Alpha strain or a sublineage thereof, does not comprise the last two Cysteine (C) residues from the transmembrane domain.

In one embodiment, the transmembrane domain of a Spike protein from SARS-CoV-2 consists of the amino acid sequence SEQ ID NO: 89.

SEQ ID NO: 89
WYIWLGFIAGLIAIVMVTIMLCC

It well known in the art that the cytosolic and transmembrane domains are highly conserved among the coronaviruses, in particular among the SARS-CoV-2 strains. Thus, the one skilled artisan in the art will not have any difficulty to define and identify the corresponding transmembrane and cytosolic domains in any coronavirus.

In one embodiment, the sequence (ii) comprises or consists of the sequence SEQ ID NO: 40 or a variant thereof sharing at least 70%, 75%, 80%, 85%, 90%, 95%, 96%, 97%, 98%, 99% or more sequence identity with said sequence.

In one embodiment, the sequence (ii) has a nucleic acid sequence coding for a S2 domain of a Spike protein comprising or consisting of SEQ ID NO: 41, or a variant thereof sharing at least 70%, 75%, 80%, 85%, 90%, 95%, 96%, 97%, 98%, 99% or more sequence identity with said sequence.

In one embodiment, the nucleic acid according to the present invention comprises:
a sequence (i) as defined hereinabove, and
a sequence (ii) as defined hereinabove.

In one embodiment, the nucleic acid according to the present invention comprises:
a sequence (i) from a Spike protein from a virus of the Orthocoronavirinae subfamily selected from the group comprising or consisting of MERS, SARS-CoV-1 and SARS-CoV-2, as defined hereinabove, and
a sequence (ii) from a SARS-CoV-2, as defined hereinabove, wherein the sequences (i) and (ii) are from (or derived from) Spike proteins from different viruses of the Orthocoronavirinae subfamily, or from Spike proteins from different strains of the same virus of the Orthocoronavirinae subfamily.

In one embodiment, the sequence (i) codes for a S1 domain comprising two parts, wherein the S1-C terminus and the N-terminal part are from (or are derived from) different Spike proteins, and wherein the S1-C terminus is from the same Spike protein from which the S2 domain is derived.

In one embodiment, the S1-C terminus is the S1-C terminus of a Spike protein from SARS-CoV-2, preferably from SARS-CoV-2 Alpha strain or a sublineage thereof.

In one embodiment, the nucleic acid according to the present invention comprises:
a sequence (i) from a Spike protein from a virus of the Orthocoronavirinae subfamily selected from the group comprising or consisting of MERS, SARS-CoV-1 and the SARS-CoV-2 Beta, Gamma, Delta and Omicron strains or sublineages thereof, as defined hereinabove, and
a sequence (ii) from a SARS-CoV-2 Alpha strain or a sublineage thereof, as defined hereinabove.

In one embodiment, the sequence (i) codes for a S1 domain comprising two parts, wherein the S1-C terminus and the N-terminal part are from (or are derived from) different Spike proteins, and wherein the S1-C terminus is from the same Spike protein from which the S2 domain is derived.

In one embodiment, the S1-C terminus is the S1-C terminus of a Spike protein from SARS-CoV-2 Alpha strain or a sublineage thereof.

In one embodiment, the nucleic acid according to the present invention comprises:
a sequence (i) from a Spike protein from a virus of the Orthocoronavirinae subfamily selected from the group comprising or consisting of MERS, SARS-CoV-1 and the SARS-CoV-2 Omicron strain or a sublineage thereof, as defined hereinabove, and
a sequence (ii) from a SARS-CoV-2 Alpha strain or a sublineage thereof, as defined hereinabove.

In one embodiment, the sequence (i) codes for a S1 domain comprising two parts, wherein the S1-C terminus and the N-terminal part are from (or are derived from) different Spike proteins, and wherein the S1-C terminus is from the same Spike protein from which the S2 domain is derived.

In one embodiment, the S1-C terminus is the S1-C terminus of a Spike protein from SARS-CoV-2 Alpha strain or a sublineage thereof.

In one embodiment, the nucleic acid according to the present invention comprises:
a sequence (i) coding for the N-terminal part of the S1 domain of a Spike protein from MERS, and a S1-C terminus of the Spike protein from SARS-CoV-2, preferably SARS-CoV-2 Alpha strain or a sublineage thereof, as defined hereinabove, and
a sequence (ii) coding for a modified S2 domain of a Spike protein from SARS-CoV-2, preferably SARS-CoV-2 Alpha strain or a sublineage thereof, as defined hereinabove, optionally wherein said S2 domain is modified by the insertion of at least one proline (P) residue, preferably two consecutive proline residues, between the Lysine (K) and Valine (V) residues in the amino acid motif RLDKV (SEQ ID NO: 39).

In one embodiment, the S1-C terminus is from the same Spike protein from which the S2 domain is derived.

In one embodiment, the nucleic acid according to the present invention comprises the sequence SEQ ID NO: 48, coding for an amino acid sequence with SEQ ID NO: 49, or a variant thereof sharing at least 70%, 75%, 80%, 85%, 90%, 95%, 96%, 97%, 98%, 99% or more sequence identity with said sequences.

In one embodiment, the nucleic acid according to the present invention comprises:
a sequence (i) coding for the N-terminal part of the S1 domain of a Spike protein from SARS-Cov-1, preferentially SARS-CoV-1 strain Tor2, and the S1-C terminus of the Spike protein from SARS-CoV-2, preferably SARS-CoV-2 Alpha strain or a sublineage thereof, as defined hereinabove, and
a sequence (ii) coding for a modified S2 domain of a Spike protein from SARS-CoV-2, preferably SARS-CoV-2 Alpha strain or a sublineage thereof, as defined hereinabove, optionally wherein said S2 domain is modified by the insertion of at least one proline (P) residue, preferably two consecutive proline residues, between the Lysine (K) and Valine (V) residues in the amino acid motif RLDKV (SEQ ID NO: 39).

In one embodiment, the S1-C terminus is from the same Spike protein from which the S2 domain is derived.

In one embodiment, the nucleic acid according to the present invention comprises the sequence SEQ ID NO: 50, coding for an amino acid sequence with SEQ ID NO: 51, or a variant thereof sharing at least 70%, 75%, 80%, 85%, 90%, 95%, 96%, 97%, 98%, 99% or more sequence identity with said sequences.

In one embodiment, the nucleic acid according to the present invention comprises:
a sequence (i) coding for the N-terminal part of the S1 domain of a Spike protein from SARS-CoV-2, preferentially SARS-CoV-2 Omicron strain or a sublineage thereof, and the S1-C terminus of the Spike protein from SARS-CoV-2, preferably SARS-CoV-2 Alpha strain or a sublineage thereof, as defined hereinabove, and
a sequence (ii) coding for a modified S2 domain of a Spike protein from SARS-CoV-2, preferably SARS-CoV-2 Alpha strain or a sublineage thereof, as defined hereinabove, optionally wherein said S2 domain is modified by the insertion of at least one proline (P) residue, preferably two consecutive proline residues, between the Lysine (K) and Valine (V) residues in the amino acid motif RLDKV (SEQ ID NO: 39),
   wherein the sequences (i) and (ii) are from (or derived from) Spike proteins from different strains of SARS-CoV-2.

In one embodiment, the S1-C terminus is from the same Spike protein from which the S2 domain is derived.

In one embodiment, the nucleic acid according to the present invention comprises the sequence SEQ ID NO: 52, coding for an amino acid sequence with SEQ ID NO: 53, or a variant thereof sharing at least 70%, 75%, 80%, 85%, 90%, 95%, 96%, 97%, 98%, 99% or more sequence identity with said sequence.

In one embodiment, the nucleic acid according to the present invention further comprises a sequence coding for a linker between the sequence (ii) and the sequence (iii).

In one embodiment, the sequence coding for a linker comprises or consists of the nucleic acid sequence TCTAGAGGC, coding for the amino acid sequence SRG, or a variant thereof sharing at least 70%, 75%, 80%, 85%, 90%, 95%, 96%, 97%, 98%, 99% or more sequence identity with said sequences.

According to the present invention, the nucleic acid comprises a sequence (iii) coding for a pilot peptide which interacts with ESCRT proteins.

By “pilot peptide which interacts with ESCRT proteins”, it is meant a polypeptide which allows the targeting of a peptide or polypeptide of interest with which it is associated, to membrane vesicles, in particular to exosomes.

Pilot peptides which interact with ESCRT proteins have been described in granted patents EP 2 268 816 and U.S. Pat. No. 9,546,371, which are incorporated by reference.

In one embodiment, the pilot peptide is capable of being addressed to the membrane vesicles, in particular to the exosome-forming vesicles, or to the cell compartment(s) involved in the formation of the membrane vesicles, and in particular the exosome-forming vesicles in eukaryotic cells. When integrated into a chimeric protein, such as a chimeric protein comprising an amino acid sequence of the Spike protein or any of the variants thereof defined hereinabove, fused or linked with said pilot peptide, the pilot peptide enables addressing said chimeric protein to the membrane vesicles and/or to their location(s) of formation and in particular to address said chimeric protein to the membrane of membrane vesicles, such that said protein can be secreted by a cell in association with the membrane vesicles (in particular exosomes), in particular an appropriate eukaryotic cell.

In one embodiment, the pilot peptide comprises at least one YxxL motif, in which “x” represents any amino acid residue. In particular, it may comprise one, two or three YxxL motifs.

In one embodiment, said YxxL motif or one of the YxxL motifs of the pilot peptide may, for example, be YINL (SEQ ID NO: 55) or YSHL (SEQ ID NO: 56).

In one embodiment, the pilot peptide comprises a DYxxL motif, in which “x” represents any residue.

In one embodiment, said DYxxL motif may, for example, be DYINL (SEQ ID NO: 58).

Alternatively or additionally, the pilot peptide comprises at least one motif equivalent to a YxxL motif, for example, a YxxF motif, in which “x” represents any residue.

Alternatively or additionally, the pilot peptide comprises at least one motif equivalent to a DYxxL motif, for example, a DYxxF motif, in which “x” represents any residue.

In one embodiment, the pilot peptide further comprises at least one PxxP motif, in which “x” represents any residue. In particular, it may comprise one, two, three or four PxxP motifs.

In one embodiment, said PxxP motif or one of the PxxP motifs of the pilot peptide is PSAP (SEQ ID NO: 62) or PTAP (SEQ ID NO: 63).

In one embodiment, the pilot peptide comprises at least one YxxL motif or DYxxL motif, and at least one PxxP motif.

In one embodiment, the pilot peptide consists of an amino acid sequence having one, two or three YxxL motif(s); and one, two, three or four PxxP motif(s). In one embodiment, the pilot peptide consists of an amino acid sequence having three YxxL motifs; and four PxxP motifs.

In one embodiment, the YxxL motif or one of the YxxL motifs is located downstream, i.e., in a C-terminal position, with respect to the one or more PxxP motif(s).

The proteins having a pilot peptide comprising at least one YxxL motif include cellular proteins and viral proteins. In particular, these viral proteins are proteins of enveloped viruses, such as transmembrane glycoproteins of enveloped viruses, or herpesvirus proteins, e.g., the LMP2-A protein of the Epstein-Barr virus which comprises at least two YxxL motifs.

In one embodiment, the pilot peptide is that of a transmembrane glycoprotein of a retrovirus. In one embodiment, the pilot peptide may be that of a transmembrane glycoprotein of a retrovirus selected from the group comprising or consisting of bovine leukemia virus (BLV), human immunodeficiency virus (HIV) (such as, without limitation, HIV-1 or HIV-2), human T-cell leukemia virus (HTLV) (such as, without limitation, HTLV-1 or HTLV-2), and Mason-Pfizer monkey virus (MPMV).

In one embodiment, the pilot peptide comprises one of the following amino acid sequences:

SEQ ID NO: 64:
PxxPxxxxPxxPxSxYxxLxPxxPExYxxLxPxxPDYxxL;
SEQ ID NO: 65:
PxxPxₙPxxPxₙSxYxxLxₙPxxPExₙYxxLxnPxxPDYxxL;
SEQ ID NO: 66:
PxxPxxxxPxxPxSxYxxLxPxxPExYxxLxPxxPDYxxLxxxx;
and
SEQ ID NO: 67:
PxxPxₙPxxPxₙSxYxxLxₙPxxPExₙYxxLxₙPxxPDYxxLxxxx;

in which “x” and “xₙ”, respectively, represent any residue and any one or several amino acid residue(s).

In one embodiment, the pilot peptide comprises one of the following amino acid sequences:

SEQ ID NO: 68:
PxxPxxxxxxxxxxxxYxxL;
SEQ ID NO: 69:
PxxPxxxxxxxxxxxDYxxL;
SEQ ID NO: 70:
PxxPxxYxxxxxxxxxYxxL;
SEQ ID NO: 71:
PxxPxxYxxxxxxxxDYxxL;
SEQ ID NO: 72:
PxxPExYxxLxPxxPDYxxL;
SEQ ID NO: 73:
PxxPxₙYxxL;
SEQ ID NO: 74:
PxxPxₙDYxxL;
SEQ ID NO: 75:
PxxPxₙYXₙYxxL;
SEQ ID NO: 76:
PxxPxₙYxₙDYxxL;
SEQ ID NO: 77:
PxxPEXₙYxxLxₙPxxPDYxxL;
SEQ ID NO: 78:
PxxPxxxxPxxPxxxYxxLxPxxPExYxxLxPxxPDYxxL;
SEQ ID NO: 79:
PxxPxₙPxxPxₙYxxLxₙPxxPEXₙYxxLxₙPxxPDYxxL;
SEQ ID NO: 80:
PxxPxxxxPxxPxxxYxxLxPxxPExYxxLxPxxPDYxxLxxxx;
and
SEQ ID NO: 81:
PxxPxₙPxxPxₙYxxLxₙPxxPEXₙYxxLxₙPxxPDYxxLxxxx,

in which “x” and “xₙ”, respectively, represent any residue and any one or several amino acid residue(s).

In particular, “n” may be greater than or equal to 1 and less than 50. “n” may, in particular, have any value between 1 and 20.

In one embodiment, the pilot peptide comprises from 6 to 100 amino acid residues, in particular from 20 to 80, from 30 to 70, or from 40 to 60 amino acid residues, for example 40, 41, 42, 43, 44, 45, 46, 47, 48, 49 or 50 amino acid residues.

In one embodiment, the pilot peptide comprises or consists of an amino acid sequence with SEQ ID NO: 8 (and may be referred to as “CilPP”) or a variant thereof.

In one embodiment, the nucleic acid sequence coding for the pilot peptide with SEQ ID NO: 8 comprises or consists of SEQ ID NO: 7 or a variant thereof.

SEQ ID NO: 7
GCGCCCCACTTCCCTGAAATCTCCTTCCCCCCTAAACCCGATTCTGATT
ATCAGGCCTTGCTACCATCCGCGCCAGAGATCTACTCTCACCTCTCCCC
CACCAAACCCGATTACATCAACCTTCGACCGGCGCCCTAA
SEQ ID NO: 8
APHFPEISFPPKPDSDYQALLPSAPEIYSHLSPTKPDYINLRPAP

In one embodiment, a variant of the amino acid sequence with SEQ ID NO: 8 comprises an amino acid sequence comprising at least 70%, preferably at least 75%, 80%, 85%, 90%, 95%, 96%, 97%, 98%, 99% or more contiguous amino acid residues of the amino acid sequence with SEQ ID NO: 8.

Additionally or alternatively, a variant of the amino acid sequence with SEQ ID NO: 8 comprises an amino acid sequence comprising at least 70%, preferably at least 75%, 80%, 85%, 90%, 95%, 96%, 97%, 98%, 99% or more sequence identity, preferably local sequence identity with the amino acid sequence with SEQ ID NO: 8.

In one embodiment, a variant of the amino acid sequence with SEQ ID NO: 8 retains at least one, two or three YxxL motif(s) and at least one, two, three or four PxxP motif(s). In one embodiment, a variant of the amino acid sequence with SEQ ID NO: 8 retains three YxxL motifs and four PxxP motifs.

In one embodiment, the nucleic acid according to the present invention comprises:
a sequence (i) coding for the N-terminal part of the S1 domain of a Spike protein from MERS, and a S1-C terminus of the Spike protein from SARS-CoV-2, preferably SARS-CoV-2 Alpha strain or a sublineage thereof, as defined hereinabove, and
a sequence (ii) coding for a modified S2 domain of a Spike protein from SARS-CoV-2, preferably SARS-CoV-2 Alpha strain or a sublineage thereof, optionally wherein said S2 domain is modified by the insertion of at least one proline (P) residue, preferably two consecutive proline residues, between the Lysine (K) and Valine (V) residues in the amino acid motif RLDKV (SEQ ID NO: 39), as defined hereinabove,
a linker, as defined hereinabove, and
a sequence (iii) coding for a pilot peptide, as defined hereinabove.

In one embodiment, the S1-C terminus is from the same Spike protein from which the S2 domain is derived. In one embodiment, the sequence (i) further comprises the signal peptide of the Spike protein from MERS.

In one embodiment, the nucleic acid according to the present invention comprises the sequence SEQ ID NO: 13, coding for an amino acid sequence with SEQ ID NO: 14, or a variant thereof sharing at least 70%, 75%, 80%, 85%, 90%, 95%, 96%, 97%, 98%, 99% or more sequence identity with said sequences.

In one embodiment, the nucleic acid according to the present invention comprises:
a sequence (i) coding for the N-terminal part of the S1 domain of a Spike protein from SARS-CoV-1, preferentially SARS-CoV-1 strain Tor2 or a sublineage thereof, and the S1-C terminus of the Spike protein from SARS-CoV-2, preferably SARS-CoV-2 Alpha strain or a sublineage thereof, as defined hereinabove, and
a sequence (ii) coding for a modified S2 domain of a Spike protein from SARS-CoV-2, preferably SARS-CoV-2 Alpha strain or a sublineage thereof, optionally wherein said S2 domain is modified by the insertion of at least one proline (P) residue, preferably two consecutive proline residues, between the Lysine (K) and Valine (V) residues in the amino acid motif RLDKV (SEQ ID NO: 39),
a linker, as defined hereinabove, and
a sequence (iii) coding for a pilot peptide, as defined hereinabove.

In one embodiment, the S1-C terminus is from the same Spike protein from which the S2 domain is derived. In one embodiment, the sequence (i) further comprises the signal peptide of the Spike protein from SARS-CoV-1, preferentially SARS-CoV-1 strain Tor2 or a sublineage thereof.

In one embodiment, the nucleic acid according to the present invention comprises the sequence SEQ ID NO: 11, coding for an amino acid sequence with SEQ ID NO: 12, or a variant thereof sharing at least 70%, 75%, 80%, 85%, 90%, 95%, 96%, 97%, 98%, 99% or more sequence identity with said sequences.

In one embodiment, the nucleic acid according to the present invention comprises:
a sequence (i) coding for the N-terminal part of the S1 domain of a Spike protein from SARS-CoV-2, preferentially SARS-CoV-2 Omicron strain or a sublineage thereof, and a S1-C terminal of the Spike protein from SARS-CoV-2, preferably SARS-CoV-2 Alpha strain or a sublineage thereof, as defined hereinabove, and
a sequence (ii) coding for a modified S2 domain of a Spike protein from SARS-CoV-2, preferably SARS-CoV-2 Alpha strain or a sublineage thereof, optionally wherein said S2 domain is modified by the insertion of at least one proline (P) residue, preferably two consecutive proline residues, between the Lysine (K) and Valine (V) residues in the amino acid motif RLDKV (SEQ ID NO: 39),
a linker, as defined hereinabove, and
a sequence (iii) coding for a pilot peptide, as defined hereinabove,
   wherein the sequences (i) and (ii) are from (or derived from) Spike proteins from different strains of SARS-CoV-2.

In one embodiment, the S1-C terminus is from the same Spike protein from which the S2 domain is derived. In one embodiment, the sequence (i) further comprises the signal peptide of the Spike protein from SARS-CoV-2, preferentially SARS-CoV-2 Omicron strain or a sublineage thereof.

In one embodiment, the nucleic acid according to the present invention comprises the sequence SEQ ID NO: 17, coding for an amino acid sequence with SEQ ID NO: 18, or a variant thereof sharing at least 70%, 75%, 80%, 85%, 90%, 95%, 96%, 97%, 98%, 99% or more sequence identity with said sequences.

In one embodiment, the nucleic acid according to the present invention is inserted into a nucleic acid expression vector, and is operably linked to regulatory elements.

The present invention further relates to a Spike protein from a virus of the Orthocoronavirinae subfamily, or a variant thereof, as defined hereinabove.

In one embodiment, the Spike protein comprises:
a sequence of a S1 domain of a Spike protein from MERS, as defined hereinabove, and
a sequence of a S2 domain of a Spike protein from SARS-CoV-2, preferably SARS-CoV-2 Alpha strain or a sublineage thereof, as defined hereinabove.

In one embodiment, the Spike protein comprises:
a sequence of a S1 domain of a Spike protein from MERS, wherein said S1 domain comprises two parts, wherein the S1-C terminus and the N-terminal part are from (or are derived from) different Spike proteins, as defined hereinabove, and
a sequence of a modified S2 domain of a Spike protein from SARS-CoV-2, preferably SARS-CoV-2 Alpha strain or a sublineage thereof, optionally wherein said S2 domain is modified by the insertion of at least one proline (P) residue, preferably two consecutive proline residues, between the Lysine (K) and Valine (V) residues in the amino acid motif RLDKV (SEQ ID NO: 39), as defined hereinabove.

In one embodiment, the S1-C terminus is from the same Spike protein from which the S2 domain is derived.

In one embodiment, the S1-C terminus is the S1-C terminus of a Spike protein from SARS-CoV-2, preferably from SARS-CoV-2 Alpha strain or a sublineage thereof.

In one embodiment, the Spike protein comprises:
a sequence of a S1 domain of a Spike protein from SARS-CoV-1, preferentially SARS-CoV-1 strain Tor2 or a sublineage thereof, as defined hereinabove, and
a sequence of a S2 domain of a Spike protein from SARS-CoV-2, preferably SARS-CoV-2 Alpha strain or a sublineage thereof, as defined hereinabove.

In one embodiment, the Spike protein comprises:
a sequence of a S1 domain of a Spike protein from SARS-Cov-1, preferentially SARS-CoV-1 strain Tor2 or a sublineage thereof, wherein said S1 domain comprises two parts, wherein the S1-C terminus and the N-terminal part are from (or are derived from) different Spike proteins as defined hereinabove, and
a sequence of a modified S2 domain of a Spike protein from SARS-CoV-2, preferably SARS-CoV-2 Alpha strain or a sublineage thereof, optionally wherein said S2 domain is modified by the insertion of at least one proline (P) residue, preferably two consecutive proline residues, between the Lysine (K) and Valine (V) residues in the amino acid motif RLDKV (SEQ ID NO: 39), as defined hereinabove.

In one embodiment, the S1-C terminus is from the same Spike protein from which the S2 domain is derived.

In one embodiment, the S1-C terminus is the S1-C terminus of a Spike protein from SARS-CoV-2, preferably from SARS-CoV-2 Alpha strain or a sublineage thereof.

In one embodiment, the Spike protein comprises:
a sequence of a S1 domain of a Spike protein from SARS-CoV-2, preferentially SARS-CoV-2 Omicron strain or a sublineage thereof, as defined hereinabove, and
a sequence of a S2 domain of a Spike protein from SARS-CoV-2, preferably SARS-CoV-2 Alpha strain or a sublineage thereof, as defined hereinabove, wherein the sequences (i) and (ii) are from (or derived from) Spike proteins from different strains of SARS-CoV-2.

In one embodiment, the Spike protein comprises:
a sequence of a S1 domain of a Spike protein from SARS-CoV-2, preferentially SARS-CoV-2 Omicron strain or a sublineage thereof, wherein said S1 domain comprises two parts, wherein the S1-C terminus and the N-terminal part are from (or are derived from) different Spike proteins, as defined hereinabove, and
a sequence of a modified S2 domain of a Spike protein from SARS-CoV-2, preferably SARS-CoV-2 Alpha strain or a sublineage thereof, optionally wherein said S2 domain is modified by the insertion of at least one proline (P) residue, preferably two consecutive proline residues, between the Lysine (K) and Valine (V) residues in the amino acid motif RLDKV (SEQ ID NO: 39), as defined hereinabove,
   wherein the sequences (i) and (ii) are from (or derived from) Spike proteins from different strains of SARS-CoV-2.

In one embodiment, the S1-C terminus is from the same Spike protein from which the S2 domain is derived.

In one embodiment, the S1-C terminus is the S1-C terminus of a Spike protein from SARS-CoV-2, preferably from SARS-CoV-2 Alpha strain or a sublineage thereof.

The present invention relates to a nucleic acid comprising:
(i) a sequence coding for a S1 domain of a Spike protein from a virus of the Orthocoronavirinae subfamily, or a variant thereof;
(ii) a sequence coding for a S2 domain of a Spike protein from a virus of the Orthocoronavirinae subfamily, or a variant thereof;
wherein the sequences (i) and (ii) are from (or derived from) the same Spike protein.

The present invention relates to a Spike protein from a virus of the Orthocoronavirinae subfamily, or a variant thereof, wherein said Spike protein comprises:
(i) a sequence of the S1 domain of a Spike protein from a virus of the Orthocoronavirinae subfamily, or a variant thereof, as defined hereinabove;
(ii) a sequence of the S2 domain of a Spike protein from a virus of the Orthocoronavirinae subfamily, or a variant thereof, as defined hereinabove; and
wherein the sequences (i) and (ii) are from (or derived from) the same Spike protein.

In one embodiment, the sequences (i) and (ii) are from (or are derived from) a Spike protein from a virus of the Orthocoronavirinae subfamily selected from the group comprising or consisting of Middle East respiratory syndrome-related coronavirus (MERS), Severe Acute Respiratory Syndrome Coronavirus 1 (SARS-CoV-1) and SARS-CoV-2.

In one embodiment, the sequences (i) and (ii) are from (or are derived from) a Spike protein from a virus of the Orthocoronavirinae subfamily selected from the group comprising or consisting of MERS, SARS-CoV-1 and the SARS-CoV-2 Alpha, Beta, Gamma, Delta, Omicron, Lambda, Mu, Kappa, Iota, Eta, Epsilon, Zeta and Theta strains or sublineages thereof.

In one embodiment, the sequences (i) and (ii) are from (or are derived from) a Spike protein from a virus of the Orthocoronavirinae subfamily selected from the group comprising or consisting of MERS, SARS-CoV-1 and the SARS-CoV-2 Alpha, Beta, Gamma, Delta and Omicron strains or sublineages thereof.

In one embodiment, the sequence (i) further comprises a sequence coding for a signal peptide. In one embodiment, said signal peptide is from the same Spike protein from which the S1 and S2 domains are derived (i.e. native signal peptide). In one embodiment, the signal peptide is not the native signal peptide. Examples of signal peptides are described hereinabove.

In one embodiment, the sequence (ii) codes for a modified S2 domain of a Spike protein.

In one embodiment, said modified S2 domain comprises the insertion of at least one proline (P) residue between the Lysine (K) and Valine (V) residues in the amino acid motif RLDKV (SEQ ID NO: 39).

In one embodiment, said modified S2 domain comprises the insertion of two consecutive proline (P) residues between the Lysine (K) and Valine (V) residues in the amino acid motif RLDKV (SEQ ID NO: 39).

In one embodiment, the nucleic acid comprises a sequence coding for a pilot peptide which interacts with ESCRT proteins, as defined hereinabove. In one embodiment, the nucleic acid comprises a sequence coding for a peptide linker, as defined hereinabove.

The present invention further relates to a nucleic acid comprising:
(i) a sequence coding for a S1 domain of a Spike protein from a virus of the Orthocoronavirinae subfamily, or a variant thereof;
(ii) a sequence coding for a S2 domain of a Spike protein from a virus of the Orthocoronavirinae subfamily, or a variant thereof; and
wherein the sequences (i) and (ii) are from (or derived from) the same Spike protein, as defined hereinabove, and wherein the totality or part of the cytosolic domain of the Spike protein is removed.

The present invention further relates to a Spike protein from a virus of the Orthocoronavirinae subfamily, or a variant thereof, wherein said Spike protein comprises:
(i) a sequence of the S1 domain of a Spike protein from a virus of the Orthocoronavirinae subfamily, or a variant thereof, as defined hereinabove;
(ii) a sequence of the S2 domain of a Spike protein from a virus of the Orthocoronavirinae subfamily, or a variant thereof, as defined hereinabove; and
wherein the sequences (i) and (ii) are from (or derived from) the same Spike protein, as defined hereinabove, and wherein the totality or part of the cytosolic domain of the Spike protein is removed.

In one embodiment, the S2 domain is modified to comprise the insertion of at least one proline (P) residue, preferably two consecutive proline residues, between the Lysine (K) and Valine (V) residues in the amino acid motif RLDKV (SEQ ID NO: 39).

In one embodiment, the S2 domain of a Spike protein from SARS-CoV-2, preferably SARS-CoV-2 Alpha strain or a sublineage thereof, does not comprise the totality of the cytosolic domain.

In one embodiment, the cytosolic domain of a Spike protein from SARS-CoV-2 consists of the amino acid sequence SEQ ID NO: 88.

In one embodiment, the totality or part of the transmembrane domain of the Spike protein is removed.

In one embodiment, the modified S2 domain of a Spike protein from SARS-CoV-2, preferably SARS-CoV-2 Alpha strain or a sublineage thereof, does not comprise the last two Cysteine (C) residues from the transmembrane domain.

In one embodiment, the transmembrane domain of a Spike protein from SARS-CoV-2 consists of the amino acid sequence SEQ ID NO: 89.

In one embodiment, the nucleic acid further comprises a sequence coding for a pilot peptide which interacts with ESCRT proteins, as defined hereinabove. In one embodiment, the nucleic acid comprises a sequence coding for a peptide linker, as defined hereinabove.

Examples of such modified Spike proteins are provided in the Example part, such as, for example, a Spike protein derived from SARS-CoV-2 Alpha strain with sequence SEQ ID NO: 4, coded by SEQ ID NO: 3, and a Spike protein derived from SARS-COV-2 Omicron strain with sequence SEQ ID NO: 20, coded by SEQ ID NO: 19.

In one embodiment, when the nucleic acid does not comprise a sequence coding for a pilot peptide, the nucleic acid may comprise two final codons coding for two Serine (S) residues.

Examples of such modified Spike proteins are provided in the Example part, such as, for example, a Spike protein derived from SARS-CoV-2 Alpha strain with sequence SEQ ID NO: 2, coded by SEQ ID NO: 1 and a Spike protein derived from SARS-CoV-2 Omicron strain with sequence SEQ ID NO: 22, coded by SEQ ID NO: 21.

In one embodiment, the modified S2 domain of a Spike protein from SARS-CoV-2, preferably SARS-CoV-2 Alpha strain or a sublineage thereof, does not comprise a part of the cytosolic domain.

In one embodiment, the modified S2 does not comprise a part of the cytosolic domain having the sequence KFDEDDSEPVLKGVKLHYT (SEQ ID NO: 90).

In one embodiment, the nucleic acid further comprises a sequence coding for a pilot peptide which interacts with ESCRT proteins, as defined hereinabove. In one embodiment, the nucleic acid comprises a sequence coding for a peptide linker, as defined hereinabove.

An example of such Spike protein is provided in the Example part, such as, for example, a Spike protein with sequence SEQ ID NO: 16, coded by SEQ ID NO: 15.

A further object of the present invention is an extracellular vesicle harboring at its external surface a Spike protein from a virus of the Orthocoronavirinae subfamily, or a variant thereof, as defined hereinabove.

Thus, a further object of the present invention is an extracellular vesicle, preferably an exosome, harboring at its external surface a Spike protein from a virus of the Orthocoronavirinae subfamily, or a variant thereof, wherein said Spike protein comprises:
(i) a sequence of the S1 domain of a Spike protein from a virus of the Orthocoronavirinae subfamily, or a variant thereof, as defined hereinabove;
(ii) a sequence of the S2 domain of a Spike protein from a virus of the Orthocoronavirinae subfamily, or a variant thereof, as defined hereinabove; and
wherein the sequences (i) and (ii) are from Spike proteins from a different virus of the Orthocoronavirinae subfamily, or from Spike proteins from different strains of the same virus of the Orthocoronavirinae subfamily.

Thus, a further object of the present invention is an extracellular vesicle, preferably an exosome, harboring at its external surface a Spike protein from a virus of the Orthocoronavirinae subfamily, or a variant thereof, wherein said Spike protein comprises:
(i) a sequence of the S1 domain of a Spike protein from a virus of the Orthocoronavirinae subfamily, or a variant thereof, as defined hereinabove;
(ii) a sequence of the S2 domain of a Spike protein from a virus of the Orthocoronavirinae subfamily, or a variant thereof, as defined hereinabove; and
wherein the sequences (i) and (ii) are from (or derived from) the same Spike protein, as defined hereinabove.

Thus, a further object of the present invention is an extracellular vesicle, preferably an exosome, harboring at its external surface a Spike protein from a virus of the Orthocoronavirinae subfamily, or a variant thereof, wherein said Spike protein comprises:
(i) a sequence of the S1 domain of a Spike protein from a virus of the Orthocoronavirinae subfamily, or a variant thereof, as defined hereinabove;
(ii) a sequence of the S2 domain of a Spike protein from a virus of the Orthocoronavirinae subfamily, or a variant thereof, as defined hereinabove; and
wherein the sequences (i) and (ii) are from (or derived from) the same Spike protein, and wherein the totality or part of the cytosolic domain of the Spike protein is removed, as defined hereinabove.

The expression “harboring at its external surface” means that a Spike protein is anchored, through its transmembrane domain, in the lipid bilayer of the extracellular vesicle, and exposed, partially or completely, outside said membrane vesicle, included (completely or partially) in the membrane of said extracellular vesicle.

In one embodiment, the sequence (i) is from a Spike protein from a virus of the Orthocoronavirinae subfamily selected from the group comprising or consisting of MERS, SARS-CoV-1 and SARS-CoV-2.

In one embodiment, the sequence (ii) is from a Spike protein from SARS-CoV-2.

In one embodiment, the extracellular vesicle is an exosome.

In one embodiment, exosomes have a diameter ranging from about 30 nm to about 150 nm, preferably from about 30 nm to about 120 nm, more preferably from about 40 nm to about 80 nm.

A further object of the present invention is a population of extracellular vesicles harboring at their external surface a Spike protein from a virus of the Orthocoronavirinae subfamily, or a variant thereof, as defined hereinabove.

In one embodiment, the population of extracellular vesicles according to the present invention is monodisperse in aqueous solutions, preferably in water and/or in PBS.

By “monodisperse”, it is meant that the extracellular vesicles in the population of extracellular vesicles are substantially uniform in size. By “substantially uniform”, it is meant that the extracellular vesicles have a narrow distribution of sizes around an average size. In one embodiment, the extracellular vesicles in water and/or in PBS have sizes exhibiting a standard deviation of less than 100% with respect to their average size, such as less than 75%, 50%, 40%, 30%, 20%, 10%, or less than 5%.

A further object of the present invention is a method of obtaining an extracellular vesicle or a population of extracellular vesicles harboring at its/their external surface a Spike protein from a virus of the Orthocoronavirinae subfamily, or a variant thereof, as defined hereinabove.

General means and methods for obtaining extracellular vesicles or a population of extracellular vesicles are well known in the art. See, e.g., Whitford & Guterstam, 2019. Future Med Chem. 11(10):1225-1236; Taylor & Shah, 2015. Methods. 87:3-10; Desplantes et al., 2017. Sci Rep. 7(1):1032.

In one embodiment, the method of obtaining an extracellular vesicle or a population of extracellular vesicles comprises a step of producing the extracellular vesicle or the population of extracellular vesicles, as defined hereinabove.

In one embodiment, this step of producing the extracellular vesicle or the population of extracellular vesicles comprises transfecting cells with a nucleic acid as defined hereinabove.

In one embodiment, the cells are HEK293 cells or cells from a derivative cell line. In one embodiment, the cells are immune cells, including, but not limited to, mastocytes, lymphocytes (such as, e.g., T-cells or B-cells), and dendritic cells.

In one embodiment, the method of obtaining an extracellular vesicle or a population of extracellular vesicles further comprises a step of culturing the transfected cells for a time sufficient to allow extracellular vesicle production, preferably in a medium devoid of EVs (i.e., a serum-free medium or a medium supplemented with EVs-depleted serum).

In one embodiment, the method of obtaining an extracellular vesicle or a population of extracellular vesicles further comprises a step of purifying said extracellular vesicle or population of extracellular vesicles.

In one embodiment, the step of purifying said extracellular vesicle or population of extracellular vesicles comprises clarification (such as, e.g., by centrifugation or by depth-filtration), filtration, ultra-filtration, diafiltration, size-exclusion purification and/or ion exchange chromatography of the transfected cell culture supernatant.

A further object of the invention is a composition comprising a nucleic acid comprising:
(i) a sequence coding for the S1 domain of a Spike protein from a virus of the Orthocoronavirinae subfamily, or a variant thereof;
(ii) a sequence coding for the S2 domain of a Spike protein from a virus of the Orthocoronavirinae subfamily, or a variant thereof; and
(iii) a sequence coding for a pilot peptide which interacts with ESCRT proteins,
wherein the sequences (i) and (ii) are from Spike proteins from a different virus of the Orthocoronavirinae subfamily, or from Spike proteins from different strains of the same virus of the Orthocoronavirinae subfamily, as defined hereinabove.

A further object of the invention is a composition comprising a nucleic acid comprising:
(i) a sequence coding for the S1 domain of a Spike protein from a virus of the Orthocoronavirinae subfamily, or a variant thereof;
(ii) a sequence coding for the S2 domain of a Spike protein from a virus of the Orthocoronavirinae subfamily, or a variant thereof; and
wherein the sequences (i) and (ii) are from (or derived from) the same Spike protein, as defined hereinabove.

In one embodiment, the nucleic acid comprises a sequence coding for a pilot peptide which interacts with ESCRT proteins, as defined hereinabove. In one embodiment, the nucleic acid comprises a sequence coding for a peptide linker, as defined hereinabove.

A further object of the invention is a composition comprising a nucleic acid comprising:
(i) a sequence coding for the S1 domain of a Spike protein from a virus of the Orthocoronavirinae subfamily, or a variant thereof;
(ii) a sequence coding for the S2 domain of a Spike protein from a virus of the Orthocoronavirinae subfamily, or a variant thereof; and
wherein the sequences (i) and (ii) are from (or derived from) the same Spike protein, and wherein the totality or part of the cytosolic domain of the Spike protein is removed, as defined hereinabove.

In one embodiment, the nucleic acid comprises a sequence coding for a pilot peptide which interacts with ESCRT proteins, as defined hereinabove. In one embodiment, the nucleic acid comprises a sequence coding for a peptide linker, as defined hereinabove.

A further object of the invention is a composition comprising an extracellular vesicle harboring at its external surface a Spike protein from a virus of the Orthocoronavirinae subfamily, or a variant thereof, as defined hereinabove.

A further object of the invention is a composition comprising a population of extracellular vesicles harboring at their external surface a Spike protein from a virus of the Orthocoronavirinae subfamily, or a variant thereof, as defined hereinabove.

In one embodiment, the compositions according to the present invention are pharmaceutical compositions and further comprise at least one pharmaceutically acceptable excipient.

The term “pharmaceutically acceptable excipient” includes any and all solvents, diluents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of vegetable oil saturated fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the pharmaceutical compositions comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

In one embodiment, the compositions according to the present invention do not comprise any adjuvant. In one embodiment, the compositions according to the present invention comprise at least one adjuvant.

Examples of adjuvants include, but are not limited to, cholera toxin, Escherichia coli heat-labile enterotoxin (LT), liposome, immune-stimulating complex (ISCOM), immunostimulatory sequences oligodeoxynucleotide (ISS-ODN), aluminum salts (such as, e.g., aluminum hydroxide or aluminum phosphate), Freund's complete adjuvant, Freund's incomplete adjuvant, Ribi solution, Corynebacterium parvum, Bacillus Calmette-Guérin (BCG), glucan, dextran sulfate, iron oxide, sodium alginate, and muramyl peptides.

A further object is a kit-of-parts comprising at least two parts:
(i) the first part comprising a nucleic acid as defined hereinabove; or a composition or pharmaceutical composition comprising the same; and
(ii) the second part comprising either one of:
   a. an extracellular vesicle harboring at its external surface a Spike protein from a virus of the Orthocoronavirinae subfamily, or a variant thereof, as defined hereinabove; or a composition or pharmaceutical composition comprising the same;
   b. a population of extracellular vesicles harboring at their external surface a Spike protein from a virus of the Orthocoronavirinae subfamily, or a variant thereof, as defined hereinabove; or a composition or pharmaceutical composition comprising the same.

In one embodiment, the kit-of-parts further comprises a nucleic acid comprising a sequence of a S gene coding for a Spike protein from a virus of the Orthocoronavirinae subfamily, or a variant thereof.

In one embodiment, the nucleic acid is as defined hereinabove.

In one embodiment, the nucleic acid comprises:
(i) a sequence coding for a S1 domain of a Spike protein from a virus of the Orthocoronavirinae subfamily, or a variant thereof;
(ii) a sequence coding for a S2 domain of a Spike protein from a virus of the Orthocoronavirinae subfamily, or a variant thereof; and
wherein the sequences (i) and (ii) are from (or derived from) the same Spike protein, as defined hereinabove.

In one embodiment, the nucleic acid comprises:
(i) a sequence coding for a S1 domain of a Spike protein from a virus of the Orthocoronavirinae subfamily, or a variant thereof;
(ii) a sequence coding for a S2 domain of a Spike protein from a virus of the Orthocoronavirinae subfamily, or a variant thereof; and
wherein the sequences (i) and (ii) are from (or derived from) the same Spike protein, and wherein the totality or part of the cytosolic domain of the Spike protein is removed, as defined hereinabove.

In one embodiment, the sequence (ii) codes for a modified S2 domain of a Spike protein. In one embodiment, said modified S2 domain comprises the insertion of at least one proline (P) residue between the Lysine (K) and Valine (V) residues in the amino acid motif RLDKV (SEQ ID NO: 39).

In one embodiment, said modified S2 domain comprises the insertion of two consecutive proline (P) residues between the Lysine (K) and Valine (V) residues in the amino acid motif RLDKV (SEQ ID NO: 39).

A further object of the present invention is a method of immunizing a subject against a virus of the Orthocoronavirinae subfamily.

In one embodiment, the virus of the Orthocoronavirinae subfamily is a virus of the betacoronavirus genus, as defined hereinabove. In one embodiment, the virus is selected from the group comprising or consisting of MERS, SARS-CoV-1 and SARS-CoV-2.

One of the advantages of the present invention is that the immunization of a subject with a nucleic acid or an extracellular vesicle as described hereinabove, wherein the sequences (i) and (ii) are from (or derived from) Spike proteins from different viruses of the Orthocoronavirinae subfamily, or from Spike proteins from different strains of the same virus of the Orthocoronavirinae subfamily, enables to target the evoked immune response against the S2 domain of the Spike protein, which is well conserved among the coronaviruses. Thus, said strategy may be of particular interest to develop a unique vaccine protecting against several strains of coronaviruses.

In one embodiment, the method of immunizing a subject against a virus of the Orthocoronavirinae subfamily comprises at least one step of administering to said subject at least one nucleic acid, or a variant thereof, as defined hereinabove; or a composition or pharmaceutical composition comprising the same.

In one embodiment, said step comprises the administration of a nucleic acid, as defined hereinabove. In one embodiment, said step comprises the administration of two or more nucleic acids, as defined hereinabove.

Means and methods for administering a nucleic acid to a subject (i.e., for DNA vaccine delivery) are well known in the art. Any convenient and appropriate method of delivery of the nucleic acid may be utilized. These include, e.g., delivery with cationic lipids (Goddard et al., 1997. Gene Ther. 4(11):1231-1236; Gorman et al., 1997. Gene Ther. 4(9):983-992; Chadwick et al., 1997. Gene Ther. 4(9):937-942; Gokhale et al., 1997. Gene Ther. 4(12):1289-1299; Gao & Huang, 1995. Gene Ther. 2(10):710-722), delivery by uptake of naked nucleic acid, and the like. Method of delivery of the nucleic acid can further include or be enhanced by electroporation, particle bombardment, sonoporation, magnetofection, hydrodynamic delivery and the like. Method of delivery of the nucleic acid can further include or be enhanced by the use of chemicals including, but not limited to, nucleic acid specifically modified to enhance delivery, lipoplexes, polymersomes, polyplexes, dendrimers, nanoparticles (e.g., inorganic nanoparticles), cell-penetrating peptides, cell-penetrating proteins (e.g., supercharged proteins), and the like.

In one embodiment, the method of immunizing a subject against a virus of the Orthocoronavirinae subfamily further comprises at least one step of administering to said subject either with:
a. an extracellular vesicle harboring at its external surface a Spike protein from a virus of the Orthocoronavirinae subfamily, or a variant thereof, as defined hereinabove; or a composition or pharmaceutical composition comprising the same; or
b. a population of extracellular vesicles harboring at their external surface a Spike protein from a virus of the Orthocoronavirinae subfamily, or a variant thereof, as defined hereinabove; or a composition or pharmaceutical composition comprising the same.

In one embodiment, the method of immunizing a subject against a virus of the Orthocoronavirinae subfamily comprises:
1) a first step, termed “priming step”, of administering to said subject a nucleic acid, or a variant thereof, as defined hereinabove; or a composition or pharmaceutical composition comprising the same; and
2) a second step, termed “boosting step”, of administering to said subject either of:
   a. an extracellular vesicle harboring at its external surface a Spike protein from a virus of the Orthocoronavirinae subfamily, or a variant thereof, as defined hereinabove; or a composition or pharmaceutical composition comprising the same; or
   b. a population of extracellular vesicles harboring at their external surface a Spike protein from a virus of the Orthocoronavirinae subfamily, or a variant thereof, as defined hereinabove; or a composition or pharmaceutical composition comprising the same.

In one embodiment, the method of immunizing a subject against a virus of the Orthocoronavirinae subfamily comprises:
1) at least one “priming step” of administering to said subject at least one nucleic acid, or a variant thereof, as defined hereinabove; or a composition or pharmaceutical composition comprising the same; and
2) optionally, at least one “boosting step” of administering to said subject either with:
   a. an extracellular vesicle harboring at its external surface a Spike protein from a virus of the Orthocoronavirinae subfamily, or a variant thereof as defined hereinabove; or a composition or pharmaceutical composition comprising the same; or
   b. a population of extracellular vesicles harboring at their external surface a Spike protein from a virus of the Orthocoronavirinae subfamily, or a variant thereof as defined hereinabove; or a composition or pharmaceutical composition comprising the same.

In one embodiment, the nucleic acid comprises sequences coding for S1 and S2 domains from (or derived from) Spike proteins from different viruses of the Orthocoronavirinae subfamily, or from Spike proteins from different strains of the same virus of the Orthocoronavirinae subfamily. In one embodiment, step 1) comprises the administration of a nucleic acid, as defined hereinabove. In one embodiment, step 1) comprises the administration of two or more nucleic acids, as defined hereinabove.

In one embodiment, the extracellular vesicle or the population of extracellular vesicles harbors a Spike protein, wherein the S1 and S2 domains are from (or derived from) the same Spike protein or wherein the S1 and S2 domains are from Spike proteins from a different virus of the Orthocoronavirinae subfamily, or from Spike proteins from different strains of the same virus of the Orthocoronavirinae subfamily.

In one embodiment, the method of immunizing a subject against a virus of the Orthocoronavirinae subfamily comprises:
1) at least one “priming step” of administering to said subject at least one nucleic acid, as defined hereinabove; or a composition or pharmaceutical composition comprising the same; wherein the sequences of the nucleic acid coding for S1 and S2 domains are from (or derived from) Spike proteins from different viruses of the Orthocoronavirinae subfamily, or from Spike proteins from different strains of the same virus of the Orthocoronavirinae subfamily,
2) optionally, at least one “boosting step” of administering to said subject either with:
   a. an extracellular vesicle harboring at its external surface a Spike protein from a virus of the Orthocoronavirinae subfamily, or a variant thereof as defined hereinabove; or a composition or pharmaceutical composition comprising the same; or
   b. a population of extracellular vesicles harboring at their external surface a Spike protein from a virus of the Orthocoronavirinae subfamily, or a variant thereof as defined hereinabove; or a composition or pharmaceutical composition comprising the same,
wherein the extracellular vesicle or the population of extracellular vesicles harbors a Spike protein, wherein the S1 and S2 domains are from (or derived from) the same Spike protein.

In one embodiment, the method comprises:
a priming step with two nucleic acids as defined hereinabove, wherein the sequences of the nucleic acid coding for S1 and S2 domains are from (or derived from) Spike proteins from different viruses of the Orthocoronavirinae subfamily, or from Spike proteins from different strains of the same virus of the Orthocoronavirinae subfamily,
a priming step with one nucleic acid, wherein the sequences (i) and (ii) are from (or derived from) the same Spike protein, and
a boosting step with an extracellular vesicle or a population of extracellular vesicles harboring a Spike protein, wherein the S1 and S2 domains are from (or derived from) the same Spike protein.

In one embodiment, the method of immunizing further comprises before, after or concomitantly to the steps described hereinabove, a step (c) of administering to said subject a nucleic acid comprising a sequence of a S gene coding for a Spike protein from a virus of the Orthocoronavirinae subfamily, or a variant thereof.

In one embodiment, the nucleic acid is as defined hereinabove.

In one embodiment, the nucleic acid comprises:
(i) a sequence coding for a S1 domain of a Spike protein from a virus of the Orthocoronavirinae subfamily, or a variant thereof;
(ii) a sequence coding for a S2 domain of a Spike protein from a virus of the Orthocoronavirinae subfamily, or a variant thereof; and
wherein the sequences (i) and (ii) are from (or derived from) the same Spike protein, as defined hereinabove.

In one embodiment, the nucleic acid comprises:
(i) a sequence coding for a S1 domain of a Spike protein from a virus of the Orthocoronavirinae subfamily, or a variant thereof;
(ii) a sequence coding for a S2 domain of a Spike protein from a virus of the Orthocoronavirinae subfamily, or a variant thereof; and
wherein the sequences (i) and (ii) are from (or derived from) the same Spike protein, and wherein the totality or part of the cytosolic domain of the Spike protein is removed, as defined hereinabove.

In one embodiment, the sequence (ii) codes for a modified S2 domain of a Spike protein. In one embodiment, said modified S2 domain comprises the insertion of at least one proline (P) residue between the Lysine (K) and Valine (V) residues in the amino acid motif RLDKV (SEQ ID NO: 39).

In one embodiment, said modified S2 domain comprises the insertion of two consecutive proline (P) residues between the Lysine (K) and Valine (V) residues in the amino acid motif RLDKV (SEQ ID NO: 39).

In one embodiment, said Spike protein has the totality or a part of the cytosolic domain removed. In one embodiment, said Spike protein comprises a modified S2 domain, wherein said modified S2 domain comprises the insertion of two consecutive proline (P) residues between the Lysine (K) and Valine (V) residues in the amino acid motif RLDKV (SEQ ID NO: 39), and the totality or a part of the cytosolic domain of said protein is removed. In one embodiment, said Spike protein has the totality or a part of the transmembrane domain removed.

In one embodiment, the “priming step” is to be carried out once, twice, three times, four times or more. In one embodiment, the “priming step” is to be carried out once, twice, three times, four times or more during the life of the subject. In one embodiment, the “boosting step” is to be carried out once, twice, three times, four times or more. In one embodiment, the“priming step” is to be carried out twice, and may be repeated once or more during the life of the subject, such as, for example, when no neutralizing antibodies are detected in the subject (e.g., in a serum sample of the subject).

In one embodiment, the “boosting step” is to be carried out once, twice, three times, four times or more during the life of the subject. In one embodiment, the step (c) is to be carried out once, twice, three times, four times or more. In one embodiment, the step (c) is to be carried out once, twice, three times, four times or more during the life of the subject.

In one embodiment, the “priming step” is to be carried out twice. In one embodiment, the “boosting step” is to be carried out once.

In one embodiment, the “priming step” is to be carried out twice and the “boosting step” is to be carried out once.

In one embodiment, the period of time between each iteration of the “priming step” and/or between each iteration of the “boosting step” and/or between the “priming step” and the “boosting step” ranges from about 1 day to about 6 months, preferably from about 1 week to about 3 months, more preferably from about 2 weeks to about 1 month.

In one embodiment, the period of time between each iteration of the “priming step” and/or between each iteration of the “boosting step” and/or between the “priming step” and the “boosting step” is of about 1 day, 2 days, 3 days, 4 days, 5 days, 6 days, 1 week, 2 weeks, 3 weeks, 1 month, 2 months, 3 months, 4 months, 5 months, or 6 months. In one embodiment, the period of time between each iteration of the “priming step” and/or between each iteration of the “boosting step” and/or between the “priming step” and the “boosting step” is of about 1 week, 2 weeks, 3 weeks, 1 month, 2 months, 3 months, 4 months, 5 months, or 6 months.

In one embodiment, the period of time between each iteration of the “priming step” and/or between each iteration of the “boosting step” and/or between the “priming step” and the “boosting step” is of about 3 weeks.

In one embodiment, the nucleic acid or the composition or pharmaceutical composition comprising the same, the extracellular vesicle or the population of extracellular vesicles or the composition or pharmaceutical composition comprising the same are formulated for administration to the subject.

In one embodiment, the nucleic acid or the composition or pharmaceutical composition comprising the same, the extracellular vesicle or the population of extracellular vesicles or the composition or pharmaceutical composition comprising the same is/are to be administered systemically or locally.

In one embodiment, the nucleic acid or the composition or pharmaceutical composition comprising the same, the extracellular vesicle or the population of extracellular vesicles or the composition or pharmaceutical composition comprising the same is/are to be administered by injection, orally, topically, nasally, buccally, rectally, vaginaly, intratracheally, by endoscopy, transmucosally, or by percutaneous administration.

In one embodiment, the nucleic acid or the composition or pharmaceutical composition comprising the same, the extracellular vesicle or the population of extracellular vesicles or the composition or pharmaceutical composition comprising the same is/are to be injected, preferably systemically injected.

Examples of formulations adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Examples of systemic injections include, but are not limited to, intravenous (iv) injection, subcutaneous injection, intramuscular (im) injection, intradermal (id) injection, intraperitoneal (ip), intranasal (in) injection and perfusion.

It will be understood that other suitable routes of administration are also contemplated in the present invention, and the administration mode will ultimately be decided by the attending physician within the scope of sound medical judgment.

In one embodiment, the nucleic acid or the composition or pharmaceutical composition comprising the same, the extracellular vesicle or the population of extracellular vesicles or the composition or pharmaceutical composition comprising the same is/are to be administered to the subject in need thereof in a therapeutically effective amount.

The term “therapeutically effective amount” or “therapeutically effective dose”, as used herein, refers to an amount effective, at dosages and for periods of time necessary, to achieve a desired preventive result. In particular, a therapeutically effective amount refers to an amount effective, at dosages and for periods of time necessary, to achieve (1) a humoral immune response as can be determined, e.g., by increased antibody titers against the S2 and/or the S1 domains of the Spike protein from the virus of the Orthocoronavirinae subfamily, or a variant thereof; and/or (2) a T-cell mediated immunity for the S2 and/or the S1 domains of the Spike protein from the virus of the Orthocoronavirinae subfamily, or a variant thereof; and/or (3) S2 and/or the S1 domains-specific interferon (IFN)-γ production.

It will be however understood that the dosage of the nucleic acid or the composition or pharmaceutical composition comprising the same, the extracellular vesicle or the population of extracellular vesicles or the composition or pharmaceutical composition comprising the same will be decided by the attending physician within the scope of sound medical judgment. The specific therapeutically effective dose for any particular patient will depend upon a variety of factors including the disease being prevented and the severity of the disease; the activity of the nucleic acid or the composition or pharmaceutical composition comprising the same, the extracellular vesicle “S-EV” or the population of extracellular vesicles or the composition or pharmaceutical composition comprising the same comprising the same employed; the age, body weight, general health, sex and diet of the subject; the time of administration, route of administration, and rate of excretion of the nucleic acid or the composition or pharmaceutical composition comprising the same, the extracellular vesicle or the population of extracellular vesicles or the composition or pharmaceutical composition comprising the same employed; the duration and regimen of the treatment; drugs used in combination or coincidental with the nucleic acid or the composition or pharmaceutical composition comprising the same, the extracellular vesicle or the population of extracellular vesicles or the composition or pharmaceutical composition comprising the same employed; and like factors well known in the medical arts.

Therapeutically effective human doses can be calculated from animal doses, by converting these doses to Human Equivalent Doses (HED) based on body surface area (BSA). According to the FDA “Guidance for Industry” of July 2005, the BSA of a human with a body weight of 60 kg is about 1.62 m², while the BSA of a mouse (such as, e.g., Balb/c mice used in the experimental design described below) with a body weight of 0.020 kg is about 0.007 m². To convert mouse dose in μg/kg to HED in μg/kg, the mouse dose can be divided by 12.3 (or alternatively multiplied by 0.081).

Additionally or alternatively, one skilled in the art is well aware that therapeutically effective human doses of nucleic acids for DNA vaccination typically range between about 0.1 mg and about 6 mg of nucleic acid per administration, with some studies describing doses up to about 10 mg of nucleic acid per administration.

In one embodiment, a therapeutically effective human dose of the nucleic acid or the composition or pharmaceutical composition comprising the same to be administered during the − or each one of the − “priming step” or step (c) ranges from about 0.1 μg/kg to about 50 μg/kg of nucleic acid.

In one embodiment, a therapeutically effective human dose of the nucleic acid or the composition or pharmaceutical composition comprising the same to be administered during the − or each one of the − “priming step” or step (c) is about 0.1 μg/kg±0.05 μg/kg, 0.2 μg/kg±0.05 μg/kg, 0.3 μg/kg±0.05 μg/kg, 0.4 μg/kg±0.05 μg/kg, 0.5 μg/kg±0.05 μg/kg, 0.6 μg/kg±0.05 μg/kg, 0.7 μg/kg±0.05 μg/kg, 0.8 μg/kg±0.05 μg/kg, 0.9 μg/kg±0.05 μg/kg, 1 μg/kg±0.5 μg/kg, 2 μg/kg±0.5 μg/kg, 3 μg/kg±0.5 μg/kg, 4 μg/kg±0.5 μg/kg, 5 μg/kg±0.5 μg/kg, 6 μg/kg±0.5 μg/kg, 7 μg/kg±0.5 μg/kg, 8 μg/kg±0.5 μg/kg, 9 μg/kg±0.5 μg/kg, 10 μg/kg±0.5 μg/kg, 11 μg/kg±0.5 μg/kg, 12 μg/kg±0.5 μg/kg, 13 μg/kg±0.5 μg/kg, 14 μg/kg±0.5 μg/kg, 15 μg/kg±0.5 μg/kg, 16 μg/kg±0.5 μg/kg, 17 μg/kg±0.5 μg/kg, 18 μg/kg±0.5 μg/kg, 19 μg/kg±0.5 μg/kg, 20 μg/kg±0.5 μg/kg, 21 μg/kg±0.5 μg/kg, 22 μg/kg±0.5 μg/kg, 23 μg/kg±0.5 μg/kg, 24 μg/kg±0.5 μg/kg, 25 μg/kg±0.5 μg/kg, 26 μg/kg±0.5 μg/kg, 27 μg/kg±0.5 μg/kg, 28 μg/kg±0.5 μg/kg, 29 μg/kg±0.5 μg/kg, 30 μg/kg±0.5 μg/kg, 31 μg/kg±0.5 μg/kg, 32 μg/kg±0.5 μg/kg, 33 μg/kg±0.5 μg/kg, 34 μg/kg±0.5 μg/kg, 35 μg/kg±0.5 μg/kg, 36 μg/kg±0.5 μg/kg, 37 μg/kg±0.5 μg/kg, 38 μg/kg±0.5 μg/kg, 39 μg/kg±0.5 μg/kg, 40 μg/kg±0.5 μg/kg, 41 μg/kg±0.5 μg/kg, 42 μg/kg±0.5 μg/kg, 43 μg/kg±0.5 μg/kg, 44 μg/kg±0.5 μg/kg, 45 μg/kg±0.5 μg/kg, 46 μg/kg±0.5 μg/kg, 47 μg/kg±0.5 μg/kg, 48 μg/kg±0.5 μg/kg, 49 μg/kg±0.5 μg/kg, or 50 μg/kg±0.5 μg/kg of nucleic acid.

In one embodiment, a therapeutically effective human dose of the extracellular vesicle or the population of extracellular vesicles or the composition or pharmaceutical composition comprising the same to be administered during the − or each one of the − “boosting step” ranges from about 0.1 μg/kg to about 50 μg/kg of extracellular vesicle.

In one embodiment, a therapeutically effective human dose of the extracellular vesicle or the population of extracellular vesicles or the composition or pharmaceutical composition comprising the same to be administered during the − or each one of the − “boosting step” is about 0.1 μg/kg±0.05 μg/kg, 0.2 μg/kg±0.05 μg/kg, 0.3 μg/kg±0.05 μg/kg, 0.4 μg/kg±0.05 μg/kg, 0.5 μg/kg±0.05 μg/kg, 0.6 μg/kg±0.05 μg/kg, 0.7 μg/kg±0.05 μg/kg, 0.8 μg/kg±0.05 μg/kg, 0.9 μg/kg±0.05 μg/kg, 1 μg/kg±0.5 μg/kg, 2 μg/kg±0.5 μg/kg, 3 μg/kg±0.5 μg/kg, 4 μg/kg±0.5 μg/kg, 5 μg/kg±0.5 μg/kg, 6 μg/kg±0.5 μg/kg, 7 μg/kg±0.5 μg/kg, 8 μg/kg±0.5 μg/kg, 9 μg/kg±0.5 μg/kg, 10 μg/kg±0.5 μg/kg, 11 μg/kg±0.5 μg/kg, 12 μg/kg±0.5 μg/kg, 13 μg/kg±0.5 μg/kg, 14 μg/kg±0.5 μg/kg, 15 μg/kg±0.5 μg/kg, 16 μg/kg±0.5 μg/kg, 17 μg/kg±0.5 μg/kg, 18 μg/kg±0.5 μg/kg, 19 μg/kg±0.5 μg/kg, 20 μg/kg±0.5 μg/kg, 21 μg/kg±0.5 μg/kg, 22 μg/kg±0.5 μg/kg, 23 μg/kg±0.5 μg/kg, 24 μg/kg±0.5 μg/kg, 25 μg/kg±0.5 μg/kg, 26 μg/kg±0.5 μg/kg, 27 μg/kg±0.5 μg/kg, 28 μg/kg±0.5 μg/kg, 29 μg/kg±0.5 μg/kg, 30 μg/kg±0.5 μg/kg, 31 μg/kg±0.5 μg/kg, 32 μg/kg±0.5 μg/kg, 33 μg/kg±0.5 μg/kg, 34 μg/kg±0.5 μg/kg, 35 μg/kg±0.5 μg/kg, 36 μg/kg±0.5 μg/kg, 37 μg/kg±0.5 μg/kg, 38 μg/kg±0.5 μg/kg, 39 μg/kg±0.5 μg/kg, 40 μg/kg±0.5 μg/kg, 41 μg/kg±0.5 μg/kg, 42 μg/kg±0.5 μg/kg, 43 μg/kg±0.5 μg/kg, 44 μg/kg±0.5 μg/kg, 45 μg/kg±0.5 μg/kg, 46 μg/kg±0.5 μg/kg, 47 μg/kg±0.5 μg/kg, 48 μg/kg±0.5 μg/kg, 49 μg/kg±0.5 μg/kg, 50 μg/kg±0.5 μg/kg, of extracellular vesicle.

A further object of the present invention is a method of production of neutralizing antibodies against a virus of the Orthocoronavirinae subfamily.

In one embodiment, the virus of the Orthocoronavirinae subfamily is a virus of the betacoronavirus genus, as defined hereinabove. In one embodiment, the virus is selected from the group comprising or consisting of MERS, SARS-CoV-1 and SARS-CoV-2.

In one embodiment, the method of production of neutralizing antibodies against a virus of the Orthocoronavirinae subfamily is an in vitro method. In vitro methods for producing antibodies include, without limitation, antibody phage display.

As used herein, antibody phage display is based on the genetic engineering of bacteriophages and repeated rounds of antigen-guided selection and phage propagation, which allows in vitro selection of antibodies.

In one embodiment, the in vitro method of production of neutralizing antibodies against a virus of the Orthocoronavirinae subfamily comprises the use of a nucleic acid, an extracellular vesicle or a population of extracellular vesicles as defined hereinabove.

In one embodiment, the method of production of neutralizing antibodies against a virus of the Orthocoronavirinae subfamily is an in vivo method.

In one embodiment, the method of production of neutralizing antibodies against a virus of the Orthocoronavirinae subfamily comprises a first step of administering a nucleic acid coding for a Spike protein of a virus of the Orthocoronavirinae subfamily, an extracellular vesicle or a population of extracellular vesicles harboring a Spike protein of a virus of the Orthocoronavirinae subfamily at the surface, to a subject.

In one embodiment, said method comprises the administration of at least one nucleic acid coding for a Spike protein of a virus of the Orthocoronavirinae subfamily, at least one extracellular vesicle or at least one population of extracellular vesicles harboring a Spike protein of a virus of the Orthocoronavirinae subfamily at the surface, to a subject.

In one embodiment, the nucleic acid, the extracellular vesicle or the population of extracellular vesicles is as defined hereinabove. In one embodiment, said administration is to be carried out once, twice, three times, four times or more.

In one embodiment, said subject is an animal, preferably a non-human animal. In one embodiment, the subject is a human.

The neutralizing antibodies may be chimeric, humanized or human antibodies.

Hence, the subject may be, e.g., a transgenic or transchromosomic non-human animal carrying parts of the human immune system for immunization or which have been engineered to express a human antibody repertoire.

In one embodiment, the method of production of neutralizing antibodies against a virus of the Orthocoronavirinae subfamily comprises a second step of collecting the serum of the subject. In one embodiment, said method of production of neutralizing antibodies against a virus of the Orthocoronavirinae subfamily comprises a third step of purifying the serum to collect the neutralizing antibodies.

In another embodiment, the method of production of neutralizing antibodies against a virus of the Orthocoronavirinae subfamily comprises a second step of collecting the splenocytes of the immunized subject to create hybridoma cell lines. In one embodiment, said method of production of neutralizing antibodies against a virus of the Orthocoronavirinae subfamily comprises a third step of culturing the hybridoma cell lines and a fourth step of purifying the neutralizing antibodies.

In another embodiment, the method of production of neutralizing antibodies against a virus of the Orthocoronavirinae subfamily comprises a second step of collecting the splenocytes of the immunized subject to clone the sequences of mRNA that encode antibodies or the variable domains of said antibodies. In one embodiment, the cloning of the mRNA is done with the single B lymphocyte cloning method.

As used herein, single B lymphocyte cloning method is based on immunization of subjects with the above described immunogens, isolation from blood or spleen and cloning of B-lymphocytes or B-lymphoblasts, that express antibodies that recognize specifically the Spike protein and then, cloning of the mRNA encoding the corresponding antibodies, or the variable domains of said antibodies.

In one embodiment, the method is for producing neutralizing antibodies against the S2 domain of a Spike protein of a virus of the Orthocoronavirinae subfamily, and further comprises a step of screening said antibodies.

In one embodiment, the screening comprises the steps of:
(a) contacting the antibodies with a first extracellular vesicle or a first population of extracellular vesicles harboring a Spike protein at the surface of the extracellular vesicle,
(b) selecting the antibodies which bind to the Spike protein harbored at the extracellular vesicle surface,
(c) contacting the antibodies selected in step (b) with a second extracellular vesicle or a second population of extracellular vesicles, wherein the extracellular vesicle(s) used in step (c) harbor(s) a Spike protein at the extracellular vesicle surface comprising the same S2 domain but a different S1 domain than the Spike protein harbored at the surface of the extracellular vesicle(s) used in step (a),
(d) selecting the antibodies which bind to the Spike protein harbored at the extracellular vesicle surface, thereby identifying antibodies which bind specifically to the S2 domain of a Spike protein.

In one embodiment, steps (c) and (d) are each reiterated more than once. In one embodiment, the extracellular vesicle or the population of extracellular vesicles is as defined hereinabove.

Thus, the present invention relates to a method of production of neutralizing antibodies against the S2 domain of a Spike protein of a virus of the Orthocoronavirinae subfamily, comprising the steps of:
(a) obtaining antibodies by an in vitro or an in vivo method as defined hereinabove,
(b) contacting the antibodies obtained in step (a) with a first extracellular vesicle or a first population of extracellular vesicles harboring a Spike protein at the surface of the extracellular vesicle,
(c) selecting the antibodies which bind to the Spike protein harbored at the extracellular vesicle surface,
(d) contacting the antibodies selected in step (c) with a second extracellular vesicle or a second population of extracellular vesicles, wherein the extracellular vesicle(s) used in step (d) harbor(s) a Spike protein comprising the same S2 domain but a different S1 domain than the Spike protein harbored at the surface of the extracellular vesicle(s) used in step (b),
(e) selecting the antibodies which bind to the Spike protein harbored at the extracellular vesicle surface, thereby identifying antibodies which bind specifically to the S2 domain of a Spike protein.

In one embodiment, the method is an in vivo method for producing neutralizing antibodies against the S2 domain of a Spike protein of a virus of the Orthocoronavirinae subfamily, and comprises the steps of:
(a₁) administering a nucleic acid coding for a Spike protein of a virus of the Orthocoronavirinae subfamily, an extracellular vesicle or a population of extracellular vesicles harboring a Spike protein of a virus of the Orthocoronavirinae subfamily at the surface, to a subject
(a₂) obtaining antibodies by either:
i) collecting the serum of the subject, and purifying the serum to collect the antibodies, or
ii) collecting the splenocytes of the immunized subject to create hybridoma cell lines, and culturing the hybridoma cell lines and purifying the antibodies, or
iii) collecting the splenocytes of the immunized subject to clone the mRNA sequences encoding antibodies or variable domains of said antibodies,
(b) contacting the antibodies obtained in step (a) with a first extracellular vesicle or a first population of extracellular vesicles harboring a Spike protein at the surface of the extracellular vesicle,
(c) selecting the antibodies which bind to the Spike protein harbored at the extracellular vesicle surface,
(d) contacting the antibodies selected in step (c) with a second extracellular vesicle or a second population of extracellular vesicles, wherein the extracellular vesicle(s) used in step (d) harbor(s) a Spike protein comprising the same S2 domain but a different S1 domain than the Spike protein harbored at the surface of the extracellular vesicle(s) used in step (b),
(e) selecting the antibodies which bind to the Spike protein harbored at the extracellular vesicle surface, thereby identifying antibodies which bind specifically to the S2 domain of Spike protein.

In one embodiment, steps (d) and (e) are each reiterated more than once. In one embodiment, the Spike protein coded by the nucleic acid or harbored at the extracellular vesicle surface in step (a₁) is identical to the Spike protein harbored at the extracellular vesicle surface in step (b).

A further object of the present invention is a method of screening antibodies against the S2 domain of a Spike protein of a virus of the Orthocoronavirinae subfamily comprising the steps of:
(a) contacting the antibodies to be screened with a first extracellular vesicle or a first population of extracellular vesicles harboring a Spike protein at the extracellular vesicle surface,
(b) selecting the antibodies which bind to the Spike protein harbored at the extracellular vesicle surface,
(c) contacting the antibodies selected in step (b) with a second extracellular vesicle or a second population of extracellular vesicles, wherein the extracellular vesicle(s) used in step (c) harbor(s) a Spike protein comprising the same S2 domain but a different S1 domain than the Spike protein harbored at the surface of the extracellular vesicle(s) used in step (a),
(d) selecting the antibodies which bind to the Spike protein harbored at the extracellular vesicle surface, thereby identifying antibodies which bind specifically to the S2 domain of Spike protein.

In one embodiment, steps (c) and (d) are each reiterated more than once.

In one embodiment, extracellular vesicles of step (a) harbor a Spike protein having S1 and S2 domains from Spike proteins from a different virus of the Orthocoronavirinae subfamily, or from Spike proteins from different strains of the same virus of the Orthocoronavirinae subfamily, and extracellular vesicles of step (c) harbor a Spike having either S1 and S2 domains from Spike proteins from a different virus of the Orthocoronavirinae subfamily, or from Spike proteins from different strains of the same virus of the Orthocoronavirinae subfamily; or having S1 and S2 domains from the same Spike proteins.

In one embodiment, extracellular vesicles of step (a) harbor a Spike protein having S1 and S2 domains from the same Spike proteins, and extracellular vesicles of step (c) harbor a Spike having S1 and S2 domains from Spike proteins from a different virus of the Orthocoronavirinae subfamily, or from Spike proteins from different strains of the same virus of the Orthocoronavirinae subfamily.

A further object of the present invention is a method of treating or preventing an Orthocoronavirinae infectious disease in a subject in need thereof, comprising a step of administering to said subject at least one nucleic acid, at least one extracellular vesicle, at least one population of extracellular vesicles, a composition, a pharmaceutical composition or a kit-of-parts, as described hereinabove.

A further object of the present invention is at least one nucleic acid, at least one extracellular vesicle, at least one population of extracellular vesicle, a composition, a pharmaceutical composition or a kit-of-parts as described hereinabove, for use in treating or preventing an Orthocoronavirinae infectious disease in a subject in need thereof.

A further object of the present invention is the use of at least one nucleic acid, at least one extracellular vesicle, or at least one population of extracellular vesicles for the manufacture of a medicament for treating or preventing an Orthocoronavirinae infectious disease. A further object of the present invention is the use of at least one nucleic acid and at least one extracellular vesicle or population of extracellular vesicles for the manufacture of a medicament for treating or preventing an Orthocoronavirinae infectious disease.

In one embodiment, the at least one nucleic acid, or a composition or pharmaceutical composition comprising the same, is to be administered alone to the subject in need thereof.

In one embodiment, the at least one extracellular vesicle or population of extracellular vesicle, or a composition or pharmaceutical composition comprising the same, is to be administered alone to the subject in need thereof.

In one embodiment, the at least one nucleic acid, or a composition or pharmaceutical composition comprising the same, is to be administered in combination with at least one extracellular vesicle or population of extracellular vesicle, or a composition or pharmaceutical composition comprising the same, wherein the at least one nucleic acid and the at least one extracellular vesicle or population of extracellular vesicle are to be administered sequentially.

In one embodiment, the Orthocoronavirinae infectious disease is selected from the group comprising or consisting of COVID-19, SARS, MERS, Coronavirus-induced Flu-like diseases, feline infectious peritonitis (FIP), porcine epidemic diarrhea (PED) and infections by a virus selected from the group comprising or consisting of bovine coronavirus (BCoV), and canine coronavirus (CCoV). In one embodiment, the Orthocoronavirinae infectious disease is selected from the group comprising or consisting of COVID-19, SARS and MERS.

### BRIEF DESCRIPTION OF THE DRAWINGS

FIGS. 1A-B are a set of two histograms showing the neutralizing humoral response and the cellular immune response induced by the immunization protocols 1 and 2. FIG. 1A: Neutralizing humoral response induced by protocols 1 and 2, measured by pseudovirus neutralization assay for different SARS-CoV-2 strains: SARS-CoV-2 UK strain #B.1.1.7 (UK), SARS-CoV-2 Indian strain #B.1.617 (IN), SARS-CoV-2 Brazilian strain #P.1 (BRZ) and SARS-CoV-2 South African strain #B.1.351 (SA). FIG. 1B: Antigen specific IFN-γ production induced by protocols 1 and 2, measured by ELISpot assay.

FIG. 2 is a histogram showing the neutralizing humoral response induced by protocols 3 and 4, measured by pseudovirus neutralization assay for different SARS-CoV-2 strains.

FIG. 3 is a combination of schemas showing the schematic representation of the following molecular constructs: SARS2-UK-CilPP, SARS1/SARS2-UK-CilPP, MERS/SARS2-UK-CilPP, SARS2-Om/UK-CilPP, SARS2-Om-CilPP, SARS2-Om4C-CilPP, SARS2-Om-Stop and SARS2-UK-Stop.

### EXAMPLES

The present invention is further illustrated by the following examples.

### Materials and Methods

### Expression Vectors and Molecular Cloning

Expression system to sort membrane proteins on extracellular vesicles (EVs) was developed by CILOA SAS (granted patents EP 2 268 816 and U.S. Pat. No. 9,546,371; De Gassart et al., 2009. Cell Biol Int. 33(1):36-48).

### DNA—SARS2-UK-STOP

The sequences of the modified S gene, and the modified Spike protein encoded by said gene, are provided in SEQ ID NO: 1 and SEQ ID NO: 2, respectively.

Briefly, the S gene from SARS-CoV-2 United Kingdom strain #B.1.1.7, encoding the Spike protein, was codon-optimized for human cells, and further modified as follows:
Substitution of the native Spike signal peptide by the signal peptide of the metabotropic glutamate receptor 5 (mGluR5 or GlutR5) (nucleic acid sequence with SEQ ID NO: 5, corresponding to the amino acid sequence with SEQ ID NO: 6);
Removal of the cytosolic domain, and
Insertion of two consecutive prolines in S2 domain, between the Lysine (K) and Valine (V) residues in the amino acid motif RLDKV (SEQ ID NO: 39).

### DNA^{EV}—SARS2-UK-CilPP

The sequences of the modified S gene, and the modified Spike protein encoded by said gene, are provided in SEQ ID NO: 3 and SEQ ID NO: 4, respectively.

Briefly, the S gene from SARS-CoV-2 United Kingdom strain #B.1.1.7, encoding the Spike protein, was codon-optimized for human cells, and further modified as follows:
Substitution of the native Spike signal peptide by the signal peptide of the metabotropic glutamate receptor 5 (nucleic acid sequence with SEQ ID NO: 5, corresponding to the amino acid sequence with SEQ ID NO: 6); and
Insertion of two consecutive prolines in S2 domain, between the Lysine (K) and Valine (V) residues in the amino acid motif RLDKV (SEQ ID NO: 39).

This modified S gene was cloned in PacI-NotI in an in-house pCilA-DC™ eukaryotic expression vector, in order to generate a “DNA^{S-EV}” vector. pCilA-DC™ comprises a cytomegalovirus-human T-lymphotropic virus type I (CMV-HTLV-I) promoter upstream of the PacI-NotI expression cassette; and a bGH poly(A) and Ad2 VA1 sequence downstream of the PacI-NotI expression cassette.

This modified S gene was fused in C-terminus to a pilot peptide (herein named “CilPP”) which sorts the Spike protein to the surface of EVs (nucleic acid sequence with SEQ ID NO: 7, encoding CilPP with amino acid sequence SEQ ID NO: 8), through a 3-amino acid linker (nucleic acid sequence TCTAGAGGC, encoding the linker with amino acid sequence SRG).

### DNA^{EV}—SARS1/SARS2-UK-CilPP

The sequences of the modified S gene, and the modified Spike protein encoded by said gene, are provided in SEQ ID NO: 11 and SEQ ID NO: 12, respectively.

Briefly, the part of the S gene coding the signal peptide and S1 domain of SARS-CoV-1 strain Tor2 was fused to the part of the S gene coding for the S1 C-terminus and S2 domain of SARS-CoV-2 United Kingdom strain #B.1.1.7. As mentioned hereinabove, two consecutive prolines were added in S2 domain, between the Lysine (K) and Valine (V) residues in the amino acid motif RLDKV (SEQ ID NO: 39).

Then, the modified S gene was cloned and fused in C-terminus to a pilot peptide as described hereinabove.

### DNA^{EV}—MERS/SARS2-UK-CilPP

The sequences of the modified S gene, and the modified Spike protein encoded by said gene, are provided in SEQ ID NO: 13 and SEQ ID NO: 14, respectively.

Briefly, the part of the S gene coding the signal peptide and S1 domain of MERS was fused to the part of the S gene coding for the S1 C-terminus and S2 domain of SARS-CoV-2 United Kingdom strain #B.1.1.7. As mentioned hereinabove, two consecutive prolines were added in S2 domain, between the Lysine (K) and Valine (V) residues in the amino acid motif RLDKV (SEQ ID NO: 39).

Then, the modified S gene was cloned and fused in C-terminus to a pilot peptide as described hereinabove.

### DNA^{EV}—SARS2-Om4C-CilPP

The sequences of the modified S gene, and the modified Spike protein encoded by said gene, are provided in SEQ ID NO: 15 and SEQ ID NO: 16, respectively.

Briefly, the modified S gene was obtained from the S gene coding for the signal peptide and S1 and S2 domains of SARS-CoV-2 Omicron with part of its cytosolic domain. As mentioned hereinabove, two consecutive prolines were added in S2 domain, between the Lysine (K) and Valine (V) residues in the amino acid motif RLDKV (SEQ ID NO: 39).

Then, the modified S gene was cloned and fused in C-terminus to a pilot peptide as described hereinabove.

### DNA^{EV}—SARS2-Om/UK-CilPP

The sequences of the modified S gene, and the modified Spike protein encoded by said gene, are provided in SEQ ID NO: 17 and SEQ ID NO: 18, respectively.

Briefly, the part of the S gene coding the signal peptide and S1 domain of SARS-CoV-2 Omicron was fused to the part of the S gene coding for the S1 C-terminus and S2 domain of SARS-CoV-2 United Kingdom strain #B.1.1.7. As mentioned hereinabove, two consecutive prolines were added in S2 domain, between the Lysine (K) and Valine (V) residues in the amino acid motif RLDKV (SEQ ID NO: 39).

Then, the modified S gene was cloned and fused in C-terminus to a pilot peptide as described hereinabove.

### DNA^{EV}—SARS2-Om-CilPP

The sequences of the modified S gene, and the modified Spike protein encoded by said gene, are provided in SEQ ID NO: 19 and SEQ ID NO: 20, respectively.

Briefly, the modified S gene was obtained from the S gene coding for the signal peptide and S1 and S2 domains of SARS-CoV-2 Omicron without its cytosolic domain.

As mentioned hereinabove, two consecutive prolines were added in S2 domain, between the Lysine (K) and Valine (V) residues in the amino acid motif RLDKV (SEQ ID NO: 39).

Then, the modified S gene was cloned and fused in C-terminus to a pilot peptide as described hereinabove.

### DNA—SARS2-Om-Stop

The sequences of the modified S gene, and the modified Spike protein encoded by said gene, are provided in SEQ ID NO: 21 and SEQ ID NO: 22, respectively.

Briefly, the S gene from SARS-CoV-2 Omicron, encoding the Spike protein, was codon-optimized for human cells, and further modified as follows:
Removal of the cytosolic domain, and
Insertion of two consecutive prolines in S2 domain, between the Lysine (K) and Valine (V) residues in the amino acid motif RLDKV (SEQ ID NO: 39).

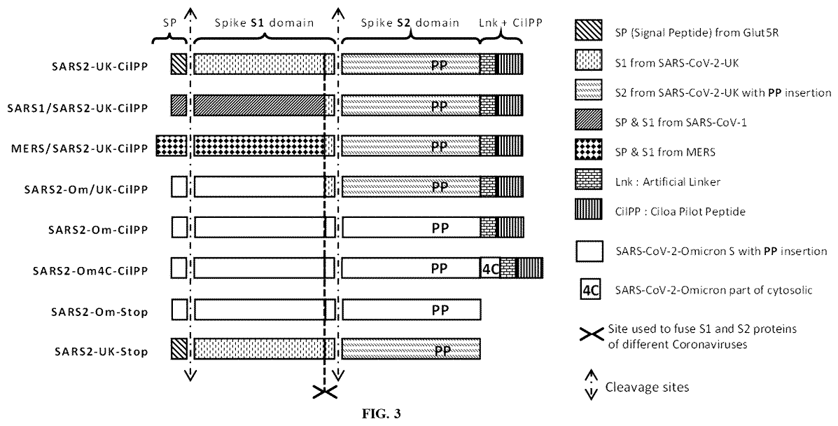

Schematic representations of the modified Spike proteins mentioned hereinabove are provided in FIG. 3.

### Production of EVs Harboring Spike Protein (S-EVs) in Mammalian Cells

EVs were produced in HEK293T cells obtained from American Type Culture Collection (ATCC). Cells were cultured in DMEM supplemented with 5% heat-inactivated fetal bovine serum (iFBS), 2 mM GlutaMAX and 5 μg/mL gentamicin at 37° C. in a 5% CO2 humidified incubator. HEK293T cells were routinely tested and found negative by MycoAlert™ mycoplasma detection kit (Lonza Nottingham, Ltd.).

DNA^{EV} were transfected into HEK293T cells using PEI. In order to generate large-scale exosome production, HEK293T cells were plated into cell chambers of 10 trays in 1 L of complete medium. Twenty-four hours post-transfection, cultures were fed with medium supplemented with EV-free iFBS and incubated for a further 48 hours.

### EVs Purification

Cell culture medium was harvested from transiently transfected HEK293T cells and S-EV isolation was performed as previously described (Taylor & Shah, 2015. Methods. 87:3-10; Desplantes et al., 2017. Sci Rep. 7(1):1032). Briefly, cell culture supernatant was clarified by two consecutive centrifugations: 10 minutes at 1300 rpm and 15 minutes at 4000 rpm, both at 4° C., followed by filtration through 0.22 μm membrane filters.

The supernatant was then concentrated by ultra-filtration and diafiltration and load onto size exclusion chromatography (SEC) columns (Sephacryl S1000, GE Healthcare). Fractions containing EVs biomarkers (CD81 and CD63) were identified by ELISA. EV fractions containing Spike protein identified by Western-blot were pooled, concentrated when necessary, and used for analysis and injections.

### Mice and Study Design

Female BALB/cAnNCrl mice, 6 weeks old, were purchased from Charles River (Italy) and placed into four groups of n=6. Mice were housed in individually ventilated cages with controlled environmental parameters: 24° C., 12 hours/12 hours light/dark cycle, nesting cotton squares for enrichment, free access to standard A04 irradiated food (SAFE, R04-25) and tap water. Behavior and health status were observed daily and weight checked weekly.

The four tested protocols are provided in the Table 1 below.

TABLE 1

| | Protocol 1 | Protocol 2 | Protocol 3 | Protocol 4 |
|---|---|---|---|---|
| Prime | DNA: | DNA^{EV}: | — | DNA^{EV}: |
| immunization | SARS2- | SARS2-UK- | | SARS1/ |
| (D 0) | UK-Stop | CilPP | | SARS2-UK- |
| | | | | CilPP |
| | | | | MERS/ |
| | | | | SARS2-UK- |
| | | | | CilPP |
| Prime | DNA: | DNA^{EV}: | DNA^{EV}: | DNA^{EV}: |
| immunization | SARS2- | SARS2-UK- | SARS2- | SARS2- |
| (D 21) | UK-Stop | CilPP | UK-CilPP | UK-CilPP |
| Boost | DNA: | EVs: | EVs: | EVs: |
| immunization | SARS2- | SARS2-UK- | SARS2- | SARS2-UK- |
| (D 42) | UK-Stop | CilPP | UK-CilPP | CilPP |

One week after their arrival, animals received 2 prime immunizations at day 0 and 21 and a boost immunization at day 42 for protocols 1, 2 and 4 or only received a prime immunization at day 21 and a boost immunization at day 42 for protocol 3.

The detailed procedure of administration of the products for the four protocols is provided hereinbelow:
group DNA^{EV}/EV (protocols 2, 3 and 4):
   prime immunization: DNA^{EV} vectors were injected using a Gene Gun (BioRad, Helios) into the abdomen of mice previously shaved. Each mouse received 3 cartridges coated with gold beads containing DNA^{E}v corresponding to a total of 3 μg of DNA, at day 0 and/or 21;
   boost immunization: 10 μg of EVs were injected subcutaneously in each mouse, in 100 μL volume of PBS, at day 42.
group DNA (protocol 1): each mouse received a total of 3 μg of DNA at days 0, 21 and 42.

Sera were collected via submandibular bleeding before each immunization: 70 to 100 mL of blood was collected with GOLDENROD animal Lancets (3 mm, Genobios).

All animals were euthanized at day 63, were exsanguinated to obtain all their sera, and spleens were collected for cellular response analysis.

### IFN-γ ELISpot Assay

Single cell suspensions were prepared from spleens of mice euthanized at day 63. Splenocytes from all immunized mice were analyzed in to pools of six animals. Total T-cells were isolated (EasySep™ Mouse T Cell Isolation Kit, StemCells #19851), and 2×10⁵ T-cells were stimulated for 18 hours in cell culture medium (RPMI 1640 with L-glutamine, 25 mM Hepes, 10% FBS and 5 μg/mL gentamycin) at 37° C. and 5% CO₂ with overlapping peptide pools representative of either S1 or S2 subunit of the Spike protein of SARS-CoV-2 (PepMix™ SARS-CoV-2 Spike glycoprotein, JPT, #PM-WCPV-S) at 1 μg/mL each, or DMSO in 96-well ELISpot IFN-γ plates (Mabtech #3321-4APW-2) in triplicates. As positive control stimuli, 0.5-1×10⁵ cells were stimulated with PHA-L (eBioscience, #15556286) at 1.25-5 μg/mL.

Following the 18-hour incubation, plates were treated according to the manufacturer's protocol. Acquisition and analysis were performed with a CTL Immunospot S6 analyzer.

Measurement values from DMSO-stimulated cells were subtracted from all the measurements. Negative values were corrected to 0. Results are represented as a number of spot forming cells (SPC) per 1×10⁶ T-cells.

### Pseudovirus Neutralization Assay

To produce SARS-CoV-2 pseudoviruses, SARS-CoV-2 truncated Spike expression and lentiviral pNL4-3.NanoLuc vectors were co-transfected into HEK293T cells using PEI. Pseudovirus-containing supernatant was collected 48 hours after, filtered through 0.45-μm filters, aliquoted and stored at −80° C.

The Spikes sequences used in the pseudotyped Lentiviruses are the following:
SARS-CoV-2 UK strain #B.1.1.7: SEQ ID NO: 23 and 24 for the DNA and protein sequences, respectively;
SARS-CoV-2 Indian strain #B.1.617: SEQ ID NO: 25 and 26 for the DNA and protein sequences, respectively;
SARS-CoV-2 Brazilian strain #P.1: SEQ ID NO: 27 and 28 for the DNA and protein sequences, respectively; and
SARS-CoV-2 South African strain #B.1.351: SEQ ID NO: 29 and 30 for the DNA and protein sequences, respectively; and
SARS-CoV-2 Omicron strain: SEQ ID NO: 31 and 32 for the DNA and protein sequences, respectively; and
MERS: SEQ ID NO: 33 and 34 for the DNA and protein sequences, respectively; and
SARS-CoV-1 Tor2 strain: SEQ ID NO: 35 and 36 for the DNA and protein sequences, respectively.

HEK293T cells transiently transfected with either DNAs encoding ACE2 SARS-CoV receptor and TMPRSS2 protease, or DNA encoding DPP4 PMERS receptor.

Sera from all 6 immunized mice of each protocol were pooled before analyzing.

To perform the neutralization assay, 2-fold mouse sera dilutions (starting from 1:10) collected at day 63 were mixed with equal volumes of pre-tittered (1000 times the background of fluorescence) Spike-pseudotyped HIV-NanoLuc viruses (1:2), incubated for 1 hour at 37° C., 5% C02 and then added to HEK293T cells transiently expressing either ACE2 receptor and TMPRSS2 protease or DPP4 receptor, in 96-well plates (in triplicates) for 1 hour at 37° C., 5% C02. Cell culture medium was then changed. After a 48-hour incubation, cells were lysed and luciferase activity (RLU, relative light units) was measured using the Bright-Glo™ Luciferase Assay System (Promega). Background luminescence produced by cells-only controls (no pseudotyped virus) and positive luminescence produced by pseudotyped virus-infected cells were included and served to determine percentage of neutralization as 100% and 0%, respectively. Neutralization titers were defined as the sera dilutions that neutralize 50% of the virus.

### Results

### Comparison of Protocol 1 vs Protocol 2

Mice were immunized at day 0, 21 and 42 with either injections of DNA coding for a modified Spike protein from SARS-CoV-2 United Kingdom strain #B.1.1.7 without its cytosolic domain and without CilPP peptide (protocol 1), or with two injections of DNA coding for a Spike protein from SARS-CoV-2 United Kingdom strain #B.1.1.7 with CilPP peptide and one injection of extracellular vesicles expressing said Spike protein fused to CilPP (protocol 2).

The humoral immune response and the cellular immune response induced by said protocols were assessed by pseudovirus neutralization assay and ELISpot assay measuring antigen specific IFN-γ production, respectively.

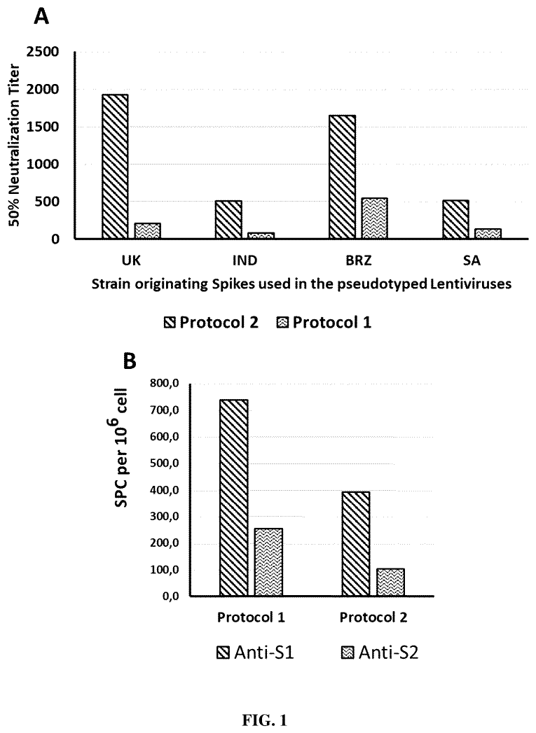

As seen in FIG. 1A, the protocol 2 induces a strong neutralizing humoral response targeting Spikes from the targeted UK strain, but also Spikes from non-targeting strains, such as the Indian and South African strains, which is not the case with the protocol 1. Thus, these results show that the protocol 2 is effective for developing neutralizing antibodies targeting Spike proteins from different SARS-CoV-2 strains.

Interestingly, mice immunized with the protocol 2 also develop a cellular immune response against both S1 and S2 domains of Spike proteins as measured by IFN-γ ELISpot, although to a lesser extent than protocol 1 (FIG. 1B).

Altogether, these results show that the protocol 2 trigger both a neutralizing humoral response and a T-cell mediated immunity, and that the neutralizing response is not limited to the coronavirus strains targeted by the immunization protocol.

### Comparison of Protocol 3 vs Protocol 4

Mice were immunized at day 0, 21 and 42 with i) one injection of DNA coding for a mix of chimeric modified Spike proteins with CilPP peptide, ii) one injection of DNA coding for a Spike protein from SARS-CoV-2 United Kingdom strain #B.1.1.7 with CilPP peptide and iii) one injection of extracellular vesicles expressing the Spike protein of ii) (protocol 4), or mice were immunized at day 21 and 42 with two injections of DNA coding for a Spike protein from SARS-CoV-2 United Kingdom strain #B.1.1.7 with CilPP peptide (protocol 3). The humoral immune response induced by said protocols was assessed by pseudovirus neutralization assay.

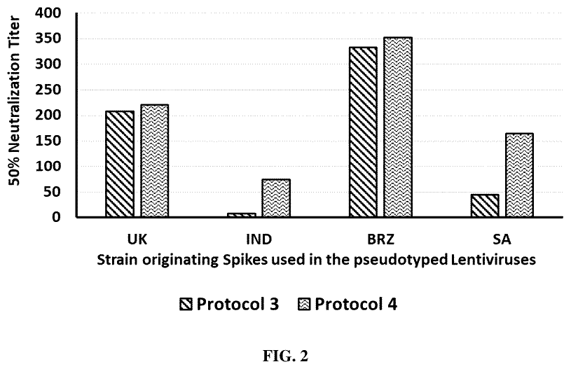

As seen in FIG. 2, the protocol 3 induces neutralizing humoral responses against the SARS-CoV-2 strains from UK and Brazil but was poorly effective in inducing humoral responses in other SARS-CoV-2 strains that were not targeted by the immunization protocol, such as, the SARS-CoV-2 strains from South Africa and India.

Interestingly, a supplementary injection of DNA coding for a mix of chimeric Spike proteins with CilPP peptide at DO did not impact the level of humoral responses against the SARS-CoV-2 strains from UK and Brazil but greatly stimulates the humoral responses against the SARS-CoV-2 strains from South Africa and India.

Thus, these results show that the use of DNA coding for a mix of chimeric Spike proteins with CilPP peptide enhances the efficacy of the humoral responses against SARS-CoV-2 strains that are not targeted by the immunization protocol.

These results strongly suggest that this injection promotes the humoral responses against the S2 domain of the Spike protein from a coronavirus. Since the S2 domain is extremely conserved among the coronaviruses, the immunization strategy presented hereinabove could be used to obtain a universal vaccine against the coronaviruses.

1-17. (canceled)

18. A nucleic acid comprising:
(i) a sequence coding for the S1 domain of a Spike protein from a virus of the Orthocoronavirinae subfamily, or a variant thereof;
(ii) a sequence coding for the S2 domain of a Spike protein from a virus of the Orthocoronavirinae subfamily, or a variant thereof; and
(iii) a sequence coding for a pilot peptide which interacts with ESCRT proteins,
wherein the sequences (i) and (ii) are from Spike proteins from a different virus of the Orthocoronavirinae subfamily, or from Spike proteins from different strains of the same virus of the Orthocoronavirinae subfamily.

19. The nucleic acid according to claim 18, wherein the sequence (ii) is from a Spike protein from Severe Acute Respiratory Syndrome Coronavirus 2 (SARS-CoV-2).

20. The nucleic acid according to claim 18, wherein the sequence (i) is from a Spike protein from a virus of the Orthocoronavirinae subfamily selected from the group comprising or consisting of Middle East respiratory syndrome related coronavirus (MERS), Severe Acute Respiratory Syndrome Coronavirus 1 (SARS-CoV-1) and SARS-CoV-2.

21. The nucleic acid according to claim 18, wherein the sequence (ii) is from a Spike protein from SARS-CoV-2 Alpha strain or a sublineage thereof, and the sequence (i) is from a Spike protein from a virus of the Orthocoronavirinae subfamily selected from the group comprising or consisting of MERS, SARS CoV 1 and the SARS-CoV2 Beta, Gamma, Delta and Omicron strains or sublineages thereof.

22. The nucleic acid according to claim 18, wherein the S2 domain of a Spike protein is modified to comprise at least one proline residue.

23. The nucleic acid according to claim 18, wherein the S2 domain of a Spike protein is modified to comprise two consecutive proline residues, between the amino acid motifs K and V in the amino acid motif RLDKV (SEQ ID NO: 39).

24. The nucleic acid according to claim 18, wherein the pilot peptide comprises at least one YxxL motif or DYxxL motif, and at least one PxxP motif, in which “x” represents any amino acid residue.

25. The nucleic acid according to claim 18, wherein the pilot peptide comprises an amino acid sequence with SEQ ID NO: 8 or a variant thereof, with the proviso that a variant of SEQ ID NO: 8 retains three YxxL motifs and four PxxP motifs, in which “x” represents any amino acid residue.

26. The nucleic acid according to claim 18, wherein the nucleic acid is inserted into a nucleic acid expression vector and operably linked to regulatory elements.

27. An extracellular vesicle harboring at its external surface a Spike protein from a virus of the Orthocoronavirinae subfamily, or a variant thereof, wherein said Spike protein comprises:
(i) a sequence of the S1 domain of a Spike protein from a virus of the Orthocoronavirinae subfamily, or a variant thereof;
(ii) a sequence of the S2 domain of a Spike protein from a virus of the Orthocoronavirinae subfamily, or a variant thereof; and
wherein the sequences (i) and (ii) are from Spike proteins from a different virus of the Orthocoronavirinae subfamily, or from Spike proteins from different strains of the same virus of the Orthocoronavirinae subfamily.

28. The extracellular vesicle according to claim 27, wherein the sequence (ii) is from a Spike protein from SARS-CoV-2.

29. The extracellular vesicle according to claim 28, wherein the sequence (i) is from a Spike protein from a virus of the Orthocoronavirinae subfamily selected from the group comprising or consisting of MERS, SARS-CoV-1 and SARS-CoV-2.

30. The extracellular vesicle according to claim 27, wherein the extracellular vesicle is obtainable by a method comprising steps of:
1) transfecting cells with a nucleic acid comprising:
(i) a sequence coding for the S1 domain of a Spike protein from a virus of the Orthocoronavirinae subfamily, or a variant thereof;
(ii) a sequence coding for the S2 domain of a Spike protein from a virus of the Orthocoronavirinae subfamily, or a variant thereof; and
(iii) a sequence coding for a pilot peptide which interacts with ESCRT proteins,
wherein the sequences (i) and (ii) are from Spike proteins from a different virus of the Orthocoronavirinae subfamily, or from Spike proteins from different strains of the same virus of the Orthocoronavirinae subfamily;
2) culturing the transfected cells for a time sufficient to allow extracellular vesicle production; and
3) purifying said extracellular vesicle.

31. A population of extracellular vesicles according to claim 27.

32. A method of immunizing a subject against a virus of the Orthocoronavirinae subfamily, said method comprising:
at least one priming step, wherein at least one nucleic acid according to claim 18 is administered to said subject,
thereby immunizing the subject against a virus of the Orthocoronavirinae subfamily.

33. The method according to claim 32, wherein said method further comprises at least one boosting step, wherein an extracellular vesicle harboring at its external surface a Spike protein from a virus of the Orthocoronavirinae subfamily, or a variant thereof, is administered to said subject, and wherein said Spike protein comprises:
(i) a sequence of the S1 domain of a Spike protein from a virus of the Orthocoronavirinae subfamily, or a variant thereof;
(ii) a sequence of the S2 domain of a Spike protein from a virus of the Orthocoronavirinae subfamily, or a variant thereof; and
wherein the sequences (i) and (ii) are from Spike proteins from a different virus of the Orthocoronavirinae subfamily, or from Spike proteins from different strains of the same virus of the Orthocoronavirinae subfamily.

34. The method according to claim 32, wherein said method further comprises at least one step of administering a nucleic acid comprising a sequence of a S gene coding for a Spike protein from a virus of the Orthocoronavirinae subfamily, or a variant thereof.

35. An in vitro method of producing neutralizing antibodies, said method comprising the use of the nucleic acid according to claim 18.

36. A method of producing neutralizing antibodies in a subject in need thereof, said method comprising at least one step of administering at least one nucleic acid according to claim 18 to the subject, thereby preventing or treating an Orthocoronavirinae infectious disease in the subject in need thereof.

37. A method of screening antibodies against the S2 domain of a Spike protein of a virus of the Orthocoronavirinae subfamily comprising the steps of:
(a) contacting the antibodies to be screened with a first extracellular vesicle, or a first population of extracellular vesicle harboring a Spike protein at the extracellular vesicle surface,
(b) selecting the antibodies which bind to the Spike protein harbored at the extracellular vesicle surface,
(c) contacting the antibodies selected in step (b) with a second extracellular vesicle or a second population of extracellular vesicle, wherein the extracellular vesicle(s) used in step (c) harbor(s) a Spike protein comprising the same S2 domain but a different S1 domain than the Spike protein harbored at the surface of the extracellular vesicle(s) used in step (a),
(d) selecting the antibodies which bind to the Spike protein harbored at the extracellular vesicle surface, thereby identifying antibodies which bind specifically to the S2 domain of Spike protein.


SEQUENCE LISTING (90 sequences)

SEQ ID NO: 1 - DNA, 3750 bp, synthetic construct
  misc_feature 1..3750: note = SARS2-UK-STOP
  source 1..3750: mol_type = other DNA
atggttctgctgctgatcctgtctgtgctgctgctcaaagaagatgtgcggggcagcgcccagtctacaggccagtgtgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagctctggatggacagctggcgccgctgcctactacgttggctatctccagccaagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgtttcggaagtccaatctgaagcccttcgagcgggacatctccaccgagatctatcaggccggcagcaccccttgtaacggcgtggaaggcttcaactgctacttcccactgcaaagctacggcttccagccaacatacggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggacattgccgataccacagatgccgtcagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggaatctgtgctagctaccagacacagaccaacagtcccagacgggccagatctgtggccagccagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataagcctcctgtggaagccgaggtgcagatcgacagactgatcaccggcagactccagagcctccagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtccggcaactgcgacgtcgtgatcggcatcgtgaacaataccgtgtacgaccctctgcaacccgagctggacagcttcaaagaggaactggacaagtacttcaagaaccacacaagccccgacgtggacctgggcgatattagcggcatcaatgccagcgtcgtgaacatccagaaagagatcgaccggctgaacgaggtggccaagaacctgaacgagagcctgatcgacctgcaagagctggggaagtacgagcagtatatcaagtggccctggtacatctggctgggctttatcgccggcctgattgccatcgtgatggtcacaatcatgctgtctagctag

SEQ ID NO: 2 - protein, 1249 aa, synthetic construct
  REGION 1..1249: note = SARS2-UK-STOP
MVLLLILSVLLLKEDVRGSAQSTGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSS

SEQ ID NO: 3 - DNA, 3888 bp, synthetic construct
  misc_feature 1..3888: note = SARS2-UK-CILPP
  source 1..3888: mol_type = other DNA
atggttctgctgctgatcctgtctgtgctgctgctcaaagaagatgtgcggggcagcgcccagtctacaggccagtgtgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagctctggatggacagctggcgccgctgcctactacgttggctatctccagccaagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgtttcggaagtccaatctgaagcccttcgagcgggacatctccaccgagatctatcaggccggcagcaccccttgtaacggcgtggaaggcttcaactgctacttcccactgcaaagctacggcttccagccaacatacggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggacattgccgataccacagatgccgtcagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggaatctgtgctagctaccagacacagaccaacagtcccagacgggccagatctgtggccagccagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataagcctcctgtggaagccgaggtgcagatcgacagactgatcaccggcagactccagagcctccagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtccggcaactgcgacgtcgtgatcggcatcgtgaacaataccgtgtacgaccctctgcaacccgagctggacagcttcaaagaggaactggacaagtacttcaagaaccacacaagccccgacgtggacctgggcgatattagcggcatcaatgccagcgtcgtgaacatccagaaagagatcgaccggctgaacgaggtggccaagaacctgaacgagagcctgatcgacctgcaagagctggggaagtacgagcagtatatcaagtggccctggtacatctggctgggctttatcgccggcctgattgccatcgtgatggtcacaatcatgctgtctagaggcgcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 4 - protein, 1295 aa, synthetic construct
  REGION 1..1295: note = SARS2-UK-CILPP
MVLLLILSVLLLKEDVRGSAQSTGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSRGAPHFPEISFPPKPDSDYQALLPSAPEIYSHLSPTKPDYINLRPAP

SEQ ID NO: 5 - DNA, 69 bp, synthetic construct
  misc_feature 1..69: note = MGLUR5 SIGNAL PEPTIDE
  source 1..69: mol_type = other DNA
atggttctgctgctgatcctgtctgtgctgctgctcaaagaagatgtgcggggcagcgcccagtctaca

SEQ ID NO: 6 - protein, 23 aa, synthetic construct
  REGION 1..23: note = MGLUR5 SIGNAL PEPTIDE
MVLLLILSVLLLKEDVRGSAQST

SEQ ID NO: 7 - DNA, 138 bp, synthetic construct
  misc_feature 1..138: note = CILPP
  source 1..138: mol_type = other DNA
gcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 8 - protein, 45 aa, synthetic construct
  REGION 1..45: note = CILPP
APHFPEISFPPKPDSDYQALLPSAPEIYSHLSPTKPDYINLRPAP

SEQ ID NO: 9 - skipped

SEQ ID NO: 10 - protein, 4 aa, synthetic construct
  REGION 1..4: note = RRAR MOTIF
RRAR

SEQ ID NO: 11 - DNA, 3813 bp, synthetic construct
  misc_feature 1..3813: note = SARS1/SARS2-UK-CILPP
  source 1..3813: mol_type = other DNA
atgtttatctttctgctgtttctgaccctgaccagcggcagcgacctggatagatgcaccaccttcgacgatgtgcaggcccctaactacacccagcacaccagctctatgcggggcgtgtactaccccgacgagatcttcagaagcgacaccctgtatctgacccaggacctgttcctgcctttctacagcaacgtgaccggcttccacaccatcaaccacaccttcggcaaccctgtgatccccttcaaggacggcatctactttgccgccaccgagaagtccaacgtcgtcagaggatgggtgttcggcagcaccatgaacaacaagagccagagcgtgatcatcatcaacaacagcaccaacgtggtcatccgggcctgcaacttcgagctgtgcgacaacccattcttcgccgtgtccaagcctatgggcacccagacacacaccatgatcttcgacaacgccttcaactgcaccttcgagtacatcagcgacgccttcagcctggacgtgtccgagaaaagcggcaacttcaagcacctgagggaattcgtgttcaagaacaaggatggcttcctgtacgtgtacaagggctaccagcctatcgacgtcgtgcgggatctgcccagcggcttcaataccctgaagcctatcttcaagctgcccctgggcatcaacatcaccaacttcagagccatcctgaccgctttcagccccgctcaggatatttggggcacaagcgccgctgcctacttcgtgggctatctgaagcccaccacctttatgctgaagtacgacgagaacggcaccatcaccgacgccgtggactgtagccaaaatcctctggccgagctgaagtgcagcgtgaagtccttcgagatcgacaagggcatctaccagaccagcaatttcagagtggtgccctccggggatgtcgtgcggttccccaatatcaccaatctgtgccccttcggcgaggtgttcaacgccaccaagtttccctctgtgtacgcctgggagcgcaagaaaatcagcaactgcgtggccgactacagcgtgctgtacaactccaccttcttcagcaccttcaagtgctacggcgtgtccgccacaaagctgaacgacctgtgcttctccaacgtgtacgccgacagcttcgtggtcaaaggcgacgacgttcggcagattgcccctggacaaacaggcgtgatcgccgattacaactacaagctgcctgacgacttcatgggctgcgtgctggcctggaacaccagaaacatcgatgccaccagcaccggcaactacaattacaagtacagatacctgcggcacggcaagctgcggccttttgagagggacatcagcaatgtgccttttagccccgacggcaagccctgcacacctcctgctctgaattgctactggcccctgaacgactacggcttttacaccaccacaggcatcggctatcagccctatagagtggtggtcctgtcctttgagctgctgaatgcccctgccacagtgtgcggacctaagctgagcaccgacctgatcaagaaccagtgcgtgaacttcaacttcaacggcctgaccggcaccggcgtgctgacaccaagcagcaagagattccagcctttccagcagttcggccgggatgtgtccgacttcacagacagcgtcagagatcccaagaccagcgagatcctggacatctccccttgtgcctttggcggagtgtccgtgatcacccctggcacaaatgcctctagcgaagtggccgtgctgtatcaggacgtgaactgcaccgatgtgtccaccgccattcacgccgatcagctgactcccgcttggcggatctatagcacaggcaacaacgtgttccagacacaagccggctgtctgatcggagccgagcatgtggataccagctacgagtgcgacatccctatcggcgctggcatctgtgctagctaccagacacagaccaacagtcccagacgggccagatctgtggccagccagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataagcctcctgtggaagccgaggtgcagatcgacagactgatcaccggcagactccagagcctccagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtccggcaactgcgacgtcgtgatcggcatcgtgaacaataccgtgtacgaccctctgcaacccgagctggacagcttcaaagaggaactggacaagtacttcaagaaccacacaagccccgacgtggacctgggcgatattagcggcatcaatgccagcgtcgtgaacatccagaaagagatcgaccggctgaacgaggtggccaagaacctgaacgagagcctgatcgacctgcaagagctggggaagtacgagcagtatatcaagtggccctggtacatctggctgggctttatcgccggcctgattgccatcgtgatggtcacaatcatgctgtctagaggcgcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 12 - protein, 1270 aa, synthetic construct
  REGION 1..1270: note = SARS1/SARS2-UK-CILPP
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSRGAPHFPEISFPPKPDSDYQALLPSAPEIYSHLSPTKPDYINLRPAP

SEQ ID NO: 13 - DNA, 4050 bp, synthetic construct
  misc_feature 1..4050: note = MERS/SARS2-UK-CILPP
  source 1..4050: mol_type = other DNA
atgatccactccgtgtttctgctgatgttcctgctgacccctaccgagagctatgtggacgtgggccccgattctgtgaagtccgcctgtatcgaagtggacatccagcagacctttttcgacaagacctggcctcggcctatcgacgtgtccaaggccgacggcatcatctaccctcaaggccggacctacagcaacatcaccattacctaccaggggctgttcccctaccaaggcgatcacggcgatatgtacgtgtactctgccggacacgccaccggcaccacacctcagaaactgttcgtggccaactacagccaggacgtgaagcagttcgccaacggctttgttgtgcggattggcgccgctgccaatagcacaggcacagtgatcatcagccctagcaccagcgccaccatcagaaagatctaccccgccttcatgctgggcagcagcgtgggcaatttcagcgacggcaagatgggcagattcttcaatcacaccctggtgctgctgcctgatggctgtggaacactgctgagagccttctactgcatcctggaacctcggagcggcaatcactgtcctgccggcaatagctacaccagcttcgccacctatcacacccctgccaccgattgctccgacggcaactacaacagaaacgccagcctgaacagcttcaaagagtacttcaacctgcggaactgcaccttcatgtacacctacaatatcaccgaggacgagatcctcgagtggttcggcatcacacagacagctcagggcgtgcacctgttcagcagcagatacgtggacctgtacggcggcaacatgttccagtttgccacactgcccgtgtacgacaccatcaagtactacagcatcatccctcacagcatccggtccatccagagcgacagaaaagcctgggccgccttctacgtgtacaagctccagcctctgaccttcctgctggactttagcgtggacggctacatcagacgggccatcgactgcggcttcaacgatctgtctcagctgcactgctcctacgagagcttcgacgtggaaagcggcgtgtacagcgtgtccagctttgaggccaagcctagcggctctgtggtggaacaagctgaaggcgtggaatgcgacttcagccctctgctgtctggcacacctcctcaggtgtacaacttcaagcggctggtgttcaccaactgcaattacaacctgaccaagctgctgagcctgttctccgtgaacgacttcacctgtagccagatcagccctgccgccattgccagcaactgttacagcagcctgatcctggactacttcagctaccctctgagcatgaagtccgatctgagcgtgtcctccgccggaccaatcagccagttcaactacaagcagtccttcagcaaccctacctgcctgattctggccaccgtgcctcacaatctgaccaccatcaccaagcctctgaagtacagctacatcaacaagtgcagcagactgctgagcgacgacagaaccgaagtgccccagctggtcaacgccaaccagtacagcccttgcgtgtccatcgtgcctagcaccgtttgggaggacggcgactactatcggaagcagctgtctcctctggaaggcggaggatggctggtggcctctggatctacagtggccatgacagagcagctccagatgggctttggcatcaccgtgcagtacggcaccgacaccaatagcgtgtgccctaagctggaattcgccaatgacaccaagatcgccagccagctgggcaactgcgtggaatactctctctacggcgttagcggcagaggcgtgttccagaattgcaccgccgtgggcgtccgccagcagagatttgtgtacgacgcctaccagaacctcgtgggctactacagcgacgacggcaattactactgcctgcgggcctgtgtgtccgtgcctgtgtctgtgatctacgacaaagagacaaagacccacgccacactgttcggcagcgtggcctgtgaacacatcagctccaccatgagccagtactccaggtctacccggtccatgctgaagcggagagacagcacatacggccctctccagacacctgtgggatgtgtgctgggcctcgtgaacagctccctgtttgtggaagattgcaagctgcccctgggccagtctctgtgtgctagctaccagacacagaccaacagtcccagacgggccagatctgtggccagccagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataagcctcctgtggaagccgaggtgcagatcgacagactgatcaccggcagactccagagcctccagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtccggcaactgcgacgtcgtgatcggcatcgtgaacaataccgtgtacgaccctctgcaacccgagctggacagcttcaaagaggaactggacaagtacttcaagaaccacacaagccccgacgtggacctgggcgatattagcggcatcaatgccagcgtcgtgaacatccagaaagagatcgaccggctgaacgaggtggccaagaacctgaacgagagcctgatcgacctgcaagagctggggaagtacgagcagtatatcaagtggccctggtacatctggctgggctttatcgccggcctgattgccatcgtgatggtcacaatcatgctgtctagaggcgcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 14 - protein, 1349 aa, synthetic construct
  REGION 1..1349: note = MERS/SARS2-UK-CILPP
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSRGAPHFPEISFPPKPDSDYQALLPSAPEIYSHLSPTKPDYINLRPAP

SEQ ID NO: 15 - DNA, 3906 bp, synthetic construct
  misc_feature 1..3906: note = SARS2-OM4C-CILPP
  source 1..3906: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatcagcggcaccaatggcaccaagagattcgacaaccccgtgctgcccttcaacgacggggtgtactttgccagcatcgagaagtccaacatcatccgcggctggatcttcggcaccacactggatagcaagacccagagcctgctgatcgtgaacaacgccaccaacgtggtcatcaaagtgtgcgagttccagttctgcaacgacccattcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgcgagcctgaggatctgcctcagggcttttctgccctggaacctctggtggatctgcccatcggcatcaacatcacccggtttcagacactgctggccctgcacagaagctacctgacacctggcgatagcagcagcggatggacagctggtgccgccgcttactacgtgggctatctccagccaagaaccttcctgctgaagtacaacgagaacggcaccatcaccgacgccgtggattgtgctctggatcccctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaatctggccccattcttcaccttcaagtgctacggcgtgtcccctaccaagctgaacgacctgtgcttcaccaatgtgtacgccgacagcttcgtgatccggggagatgaagtgcggcagattgcccctggacagaccggcaatatcgccgactacaactacaagctgcccgacgacttcaccggctgtgtgatcgcctggaatagcaacaagctggacagcaaggtgtccggcaactacaattacctgtaccggctgtttcggaagtccaatctgaagcccttcgagcgggacatcagcaccgagatctatcaggccggcaacaagccctgtaatggcgtggccggcttcaactgctacttcccactgcggagctacagcttcagacccacatacggcgtgggccaccagccttatagagtggtggtgctgagcttcgagctgctgcatgctcctgccacagtgtgcggccctaagaaaagcaccaacctcgtgaagaacaaatgcgtgaacttcaacttcaacggcctgaaaggcaccggcgtgctgaccgagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgttccagaccagagccggctgtctgattggcgccgagtatgtgaacaacagctacgagtgcgacatccccatcggagccggcatctgtgctagctaccagacacagaccaagagccacagacgggccagaagtgtggcctctcagagcatcattgcctacacaatgagcctgggcgtcgagaacagcgtggcctacagcaacaactctatcgctatccccaccaacttcaccatcagcgtgaccaccgagattctgcccgtgtccatgaccaagaccagcgtggactgcaccatgtacatctgcggcgattccaccgagtgctccaacctgctgctccagtacggcagcttctgcacccagctgaagagagccctgacagggattgccgtggaacaggacaagaacacccaagaggtgttcgcccaagtgaagcagatctacaagacccctcctatcaagtacttcggcgggttcaacttctcccagatccttccggaccctagcaagcccagcaagcggagctttatcgaggacctgctgttcaacaaagtgacactggccgacgccggctttatcaagcagtacggcgattgcctgggagatatcgccgccagggatctgatttgcgcccagaagtttaagggcctgacagtgctgcctcctctgctgacagatgagatgatcgcccagtacacaagcgccctgctggccggcacaatcacctctggatggacatttggagccggcgctgccctgcaaatcccctttgctatgcagatggcctatcggttcaacggcatcggagtgacccagaatgtgctgtacgagaaccagaagctgatcgccaaccagttcaacagcgccatcggcaagatccaggacagcctgagcagcacagcctctgctctgggaaagctccaggacgtggtcaaccacaatgctcaggccctgaacaccctggtcaagcagctgtctagcaagttcggcgccatctctagcgtgctgaatgacatcttctccaggctggacaagcctcctgtggaagccgaggtgcagatcgacagactgatcaccggtagactccagtctctccagacctacgtgacccagcagctgattagagccgccgagatcagagccagcgccaatctggctgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcaacccgagctggactccttcaaagaggaactggataagtactttaagaaccacacaagccccgacgtggacctgggcgatattagcggcatcaatgccagcgtcgtgaacatccagaaagagatcgaccggctgaacgaggtggcaaagaacctgaacgagagcctgatcgacctgcaagaactggggaagtacgagcagtacatcaagtggccttggtacatctggctgggctttatcgccggactgattgccatcgtgatggtcacaatcatgctgtgctgtatgaccagctgctgtagctgcctgaagggctgttgtagctgtggcagctgttgctctagaggcgcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 16 - protein, 1301 aa, synthetic construct
  REGION 1..1301: note = SARS2-OM4C-CILPP
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGVENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCSRGAPHFPEISFPPKPDSDYQALLPSAPEIYSHLSPTKPDYINLRPAP

SEQ ID NO: 17 - DNA, 3846 bp, synthetic construct
  misc_feature 1..3846: note = SARS2-OM/UK-CILPP
  source 1..3846: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatcagcggcaccaatggcaccaagagattcgacaaccccgtgctgcccttcaacgacggggtgtactttgccagcatcgagaagtccaacatcatccgcggctggatcttcggcaccacactggatagcaagacccagagcctgctgatcgtgaacaacgccaccaacgtggtcatcaaagtgtgcgagttccagttctgcaacgacccattcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgcgagcctgaggatctgcctcagggcttttctgccctggaacctctggtggatctgcccatcggcatcaacatcacccggtttcagacactgctggccctgcacagaagctacctgacacctggcgatagcagcagcggatggacagctggtgccgccgcttactacgtgggctatctccagccaagaaccttcctgctgaagtacaacgagaacggcaccatcaccgacgccgtggattgtgctctggatcccctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaatctggccccattcttcaccttcaagtgctacggcgtgtcccctaccaagctgaacgacctgtgcttcaccaatgtgtacgccgacagcttcgtgatccggggagatgaagtgcggcagattgcccctggacagaccggcaatatcgccgactacaactacaagctgcccgacgacttcaccggctgtgtgatcgcctggaatagcaacaagctggacagcaaggtgtccggcaactacaattacctgtaccggctgtttcggaagtccaatctgaagcccttcgagcgggacatcagcaccgagatctatcaggccggcaacaagccctgtaatggcgtggccggcttcaactgctacttcccactgcggagctacagcttcagacccacatacggcgtgggccaccagccttatagagtggtggtgctgagcttcgagctgctgcatgctcctgccacagtgtgcggccctaagaaaagcaccaacctcgtgaagaacaaatgcgtgaacttcaacttcaacggcctgaaaggcaccggcgtgctgaccgagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgttccagaccagagccggctgtctgattggcgccgagtatgtgaacaacagctacgagtgcgacatccccatcggagccggcatctgtgctagctaccagacacagaccaacagtcccagacgggccagatctgtggccagccagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataagcctcctgtggaagccgaggtgcagatcgacagactgatcaccggcagactccagagcctccagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtccggcaactgcgacgtcgtgatcggcatcgtgaacaataccgtgtacgaccctctgcaacccgagctggacagcttcaaagaggaactggacaagtacttcaagaaccacacaagccccgacgtggacctgggcgatattagcggcatcaatgccagcgtcgtgaacatccagaaagagatcgaccggctgaacgaggtggccaagaacctgaacgagagcctgatcgacctgcaagagctggggaagtacgagcagtatatcaagtggccctggtacatctggctgggctttatcgccggcctgattgccatcgtgatggtcacaatcatgctgtctagaggcgcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 18 - protein, 1281 aa, synthetic construct
  REGION 1..1281: note = SARS2-OM/UK-CILPP
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSRGAPHFPEISFPPKPDSDYQALLPSAPEIYSHLSPTKPDYINLRPAP

SEQ ID NO: 19 - DNA, 3846 bp, synthetic construct
  misc_feature 1..3846: note = SARS2-OM-CILPP
  source 1..3846: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatcagcggcaccaatggcaccaagagattcgacaaccccgtgctgcccttcaacgacggggtgtactttgccagcatcgagaagtccaacatcatccgcggctggatcttcggcaccacactggatagcaagacccagagcctgctgatcgtgaacaacgccaccaacgtggtcatcaaagtgtgcgagttccagttctgcaacgacccattcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgcgagcctgaggatctgcctcagggcttttctgccctggaacctctggtggatctgcccatcggcatcaacatcacccggtttcagacactgctggccctgcacagaagctacctgacacctggcgatagcagcagcggatggacagctggtgccgccgcttactacgtgggctatctccagccaagaaccttcctgctgaagtacaacgagaacggcaccatcaccgacgccgtggattgtgctctggatcccctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaatctggccccattcttcaccttcaagtgctacggcgtgtcccctaccaagctgaacgacctgtgcttcaccaatgtgtacgccgacagcttcgtgatccggggagatgaagtgcggcagattgcccctggacagaccggcaatatcgccgactacaactacaagctgcccgacgacttcaccggctgtgtgatcgcctggaatagcaacaagctggacagcaaggtgtccggcaactacaattacctgtaccggctgtttcggaagtccaatctgaagcccttcgagcgggacatcagcaccgagatctatcaggccggcaacaagccctgtaatggcgtggccggcttcaactgctacttcccactgcggagctacagcttcagacccacatacggcgtgggccaccagccttatagagtggtggtgctgagcttcgagctgctgcatgctcctgccacagtgtgcggccctaagaaaagcaccaacctcgtgaagaacaaatgcgtgaacttcaacttcaacggcctgaaaggcaccggcgtgctgaccgagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgttccagaccagagccggctgtctgattggcgccgagtatgtgaacaacagctacgagtgcgacatccccatcggagccggcatctgtgctagctaccagacacagaccaagagccacagacgggccagaagtgtggcctctcagagcatcattgcctacacaatgagcctgggcgtcgagaacagcgtggcctacagcaacaactctatcgctatccccaccaacttcaccatcagcgtgaccaccgagattctgcccgtgtccatgaccaagaccagcgtggactgcaccatgtacatctgcggcgattccaccgagtgctccaacctgctgctccagtacggcagcttctgcacccagctgaagagagccctgacagggattgccgtggaacaggacaagaacacccaagaggtgttcgcccaagtgaagcagatctacaagacccctcctatcaagtacttcggcgggttcaacttctcccagatccttccggaccctagcaagcccagcaagcggagctttatcgaggacctgctgttcaacaaagtgacactggccgacgccggctttatcaagcagtacggcgattgcctgggagatatcgccgccagggatctgatttgcgcccagaagtttaagggcctgacagtgctgcctcctctgctgacagatgagatgatcgcccagtacacaagcgccctgctggccggcacaatcacctctggatggacatttggagccggcgctgccctgcaaatcccctttgctatgcagatggcctatcggttcaacggcatcggagtgacccagaatgtgctgtacgagaaccagaagctgatcgccaaccagttcaacagcgccatcggcaagatccaggacagcctgagcagcacagcctctgctctgggaaagctccaggacgtggtcaaccacaatgctcaggccctgaacaccctggtcaagcagctgtctagcaagttcggcgccatctctagcgtgctgaatgacatcttctccaggctggacaagcctcctgtggaagccgaggtgcagatcgacagactgatcaccggtagactccagtctctccagacctacgtgacccagcagctgattagagccgccgagatcagagccagcgccaatctggctgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcaacccgagctggactccttcaaagaggaactggataagtactttaagaaccacacaagccccgacgtggacctgggcgatattagcggcatcaatgccagcgtcgtgaacatccagaaagagatcgaccggctgaacgaggtggcaaagaacctgaacgagagcctgatcgacctgcaagaactggggaagtacgagcagtacatcaagtggccttggtacatctggctgggctttatcgccggactgattgccatcgtgatggtcacaatcatgctgtctagaggcgcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 20 - protein, 1281 aa, synthetic construct
  REGION 1..1281: note = SARS2-OM-CILPP
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGVENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSRGAPHFPEISFPPKPDSDYQALLPSAPEIYSHLSPTKPDYINLRPAP

SEQ ID NO: 21 - DNA, 3708 bp, synthetic construct
  misc_feature 1..3708: note = SARS2-OM-STOP
  source 1..3708: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatcagcggcaccaatggcaccaagagattcgacaaccccgtgctgcccttcaacgacggggtgtactttgccagcatcgagaagtccaacatcatccgcggctggatcttcggcaccacactggatagcaagacccagagcctgctgatcgtgaacaacgccaccaacgtggtcatcaaagtgtgcgagttccagttctgcaacgacccattcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgcgagcctgaggatctgcctcagggcttttctgccctggaacctctggtggatctgcccatcggcatcaacatcacccggtttcagacactgctggccctgcacagaagctacctgacacctggcgatagcagcagcggatggacagctggtgccgccgcttactacgtgggctatctccagccaagaaccttcctgctgaagtacaacgagaacggcaccatcaccgacgccgtggattgtgctctggatcccctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaatctggccccattcttcaccttcaagtgctacggcgtgtcccctaccaagctgaacgacctgtgcttcaccaatgtgtacgccgacagcttcgtgatccggggagatgaagtgcggcagattgcccctggacagaccggcaatatcgccgactacaactacaagctgcccgacgacttcaccggctgtgtgatcgcctggaatagcaacaagctggacagcaaggtgtccggcaactacaattacctgtaccggctgtttcggaagtccaatctgaagcccttcgagcgggacatcagcaccgagatctatcaggccggcaacaagccctgtaatggcgtggccggcttcaactgctacttcccactgcggagctacagcttcagacccacatacggcgtgggccaccagccttatagagtggtggtgctgagcttcgagctgctgcatgctcctgccacagtgtgcggccctaagaaaagcaccaacctcgtgaagaacaaatgcgtgaacttcaacttcaacggcctgaaaggcaccggcgtgctgaccgagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgttccagaccagagccggctgtctgattggcgccgagtatgtgaacaacagctacgagtgcgacatccccatcggagccggcatctgtgctagctaccagacacagaccaagagccacagacgggccagaagtgtggcctctcagagcatcattgcctacacaatgagcctgggcgtcgagaacagcgtggcctacagcaacaactctatcgctatccccaccaacttcaccatcagcgtgaccaccgagattctgcccgtgtccatgaccaagaccagcgtggactgcaccatgtacatctgcggcgattccaccgagtgctccaacctgctgctccagtacggcagcttctgcacccagctgaagagagccctgacagggattgccgtggaacaggacaagaacacccaagaggtgttcgcccaagtgaagcagatctacaagacccctcctatcaagtacttcggcgggttcaacttctcccagatccttccggaccctagcaagcccagcaagcggagctttatcgaggacctgctgttcaacaaagtgacactggccgacgccggctttatcaagcagtacggcgattgcctgggagatatcgccgccagggatctgatttgcgcccagaagtttaagggcctgacagtgctgcctcctctgctgacagatgagatgatcgcccagtacacaagcgccctgctggccggcacaatcacctctggatggacatttggagccggcgctgccctgcaaatcccctttgctatgcagatggcctatcggttcaacggcatcggagtgacccagaatgtgctgtacgagaaccagaagctgatcgccaaccagttcaacagcgccatcggcaagatccaggacagcctgagcagcacagcctctgctctgggaaagctccaggacgtggtcaaccacaatgctcaggccctgaacaccctggtcaagcagctgtctagcaagttcggcgccatctctagcgtgctgaatgacatcttctccaggctggacaagcctcctgtggaagccgaggtgcagatcgacagactgatcaccggtagactccagtctctccagacctacgtgacccagcagctgattagagccgccgagatcagagccagcgccaatctggctgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcaacccgagctggactccttcaaagaggaactggataagtactttaagaaccacacaagccccgacgtggacctgggcgatattagcggcatcaatgccagcgtcgtgaacatccagaaagagatcgaccggctgaacgaggtggcaaagaacctgaacgagagcctgatcgacctgcaagaactggggaagtacgagcagtacatcaagtggccttggtacatctggctgggctttatcgccggactgattgccatcgtgatggtcacaatcatgctgtctagctag

SEQ ID NO: 22 - protein, 1235 aa, synthetic construct
  REGION 1..1235: note = SARS2-OM-STOP
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGVENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSS

SEQ ID NO: 23 - DNA, 3789 bp, synthetic construct
  misc_feature 1..3789: note = SARS-COV2-UK
  source 1..3789: mol_type = other DNA
atggttctgctgctgatcctgtctgtgctgctgctcaaagaagatgtgcggggcagcgcccagtctacaggccagtgtgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatcagcggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtgtaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagctctggatggacagctggcgccgctgcctactacgttggctatctccagccaagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaactgcgtggccgattactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgtttcggaagtccaatctgaagcccttcgagcgggacatctccaccgagatctatcaggccggcagcaccccttgtaacggcgtggaaggcttcaactgctacttcccactgcaaagctacggcttccagccaacatacggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggacatcgacgataccacagatgccgtcagagatccccagacactggaaatcctggacatcaccccttgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgtttcagaccagagccggctgtctgatcggagccgagcacgtgaacaatagctacgagtgcgacatccccatcggcgctggaatctgtgctagctaccagacacagaccaacagccacagacgggccagaagtgtggcctctcagagcatcattgcctacaccatgtctctgggcgccgagaacagcgtggcctacagcaacaactctatcgctatccccatcaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggtatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctggctagactggacaaggtggaagccgaggtgcagatcgacagactgatcaccggcagactccagagcctccagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccacacacaacaccttcgtgtccggcaactgcgacgtcgtgatcggcatcgtgaacaataccgtgtacgaccctctccagccagagctggacagcttcaaagaggaactggataagtacttcaagaaccacacaagccccgacgtggacctgggcgatattagcggcatcaatgccagcgtcgtgaacatccagaaagagatcgaccggctgaacgaggtggcaaagaacctgaacgagagcctgatcgacctgcaagagctggggaagtacgagcagtatatcaagtggccctggtacatctggctgggctttatcgccggcctgattgccatcgtgatggtcacaatcatgctgtgctgcatgaccagctgctgtagctgcctgaagggctgttgtagctgtggcagctgttgctaa

SEQ ID NO: 24 - protein, 1262 aa, synthetic construct
  REGION 1..1262: note = SARS-COV2-UK
MVLLLILSVLLLKEDVRGSAQSTGQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 25 - DNA, 3798 bp, synthetic construct
  misc_feature 1..3798: note = SARS-COV2-IN
  source 1..3798: mol_type = other DNA
atggttctgctgctgatcctgtctgtgctgctgctcaaagaagatgtgcggggcagcgcccagtctacaggccagtgtgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagctctggatggacagctggcgccgctgcctactacgttggctatctccagccaagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaactgcgtggccgattactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccgctaccggctgttccgcaagagcaacctgaagcctttcgagcgggacatctccaccgagatctatcaggccggcagcaccccttgtaatggcgtgcagggcttcaactgctacttcccactgcaaagctacggctttcagcccacaaacggcgtgggctaccagccttatagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggacattgccgataccacagatgccgtcagagatccccagacactggaaatcctggacatcaccccttgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgtttcagaccagagccggctgtctgatcggagccgagcacgtgaacaatagctacgagtgcgacatccccatcggcgctggaatctgtgctagctaccagacacagaccaactctcggcggagagccagatctgtggcctctcagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggtatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataaggtggaagccgaggtgcagatcgaccggctgattacaggcagactccagagcctccagacctacgtgacacagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgactcagcggaacttctacgagccccagatcatcaccaccgacaacaccttcgtgtccggcaactgcgacgtcgtgatcggcatcgtgaacaataccgtgtacgaccctctccagccagagctggacagcttcaaagaggaactggacaagtacttcaagaaccacacaagccccgacgtggacctgggcgatattagcggcatcaatgccagcgtcgtgaacatccagaaagagatcgacagactgaacgaggtcgccaagaacctgaacgagagcctgatcgacctgcaagagctggggaagtacgagcagtatatcaagtggccctggtacatctggctgggctttatcgccggcctgattgccatcgtgatggtcacaatcatgctgtgctgcatgaccagctgttgcagctgcctgaagggctgctgtagctgcggctcttgttgttaa

SEQ ID NO: 26 - protein, 1265 aa, synthetic construct
  REGION 1..1265: note = SARS-COV2-IN
MVLLLILSVLLLKEDVRGSAQSTGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 27 - DNA, 3798 bp, synthetic construct
  misc_feature 1..3798: note = SARS-COV2-BR
  source 1..3798: mol_type = other DNA
atggttctgctgctgatcctgtctgtgctgctgctcaaagaagatgtgcggggcagcgcccagtctacaggccagtgtgtgaacttcaccaacagaacccagctgcctagcgcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaactaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgagcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagctctggatggacagctggcgccgctgcctactacgttggctatctccagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaactgcgtggccgattactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcacaatcgccgactacaactacaagctgcccgacgacttcaccggctgtgtgattgcctggaacagcaacaacctggactccaaagtcggcggcaactacaattacctgtaccggctgtttcggaagtccaatctgaagcccttcgagcgggacatctccaccgagatctatcaggccggcagcaccccttgcaatggcgtgaagggctttaactgctacttcccactgcaaagctacggcttccagccaacatacggcgtgggctaccagccttacagagtggtggtgctgagcttcgagctgctgcatgctcctgccacagtgtgcggccctaagaaaagcaccaatctcgtgaagaacaaatgcgtcaacttcaatttcaacggcctgaccggcaccggcgtgctgacagagagcaacaagaagttcctgccattccagcagttcggccgggacattgccgataccacagatgccgtcagagatccccagacactggaaatcctggacatcaccccatgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgtttcagaccagagccggctgtctgatcggagccgagtatgtgaacaacagctacgagtgcgacatccccatcggcgctggaatctgtgctagctaccagacacagacaaacagccccagacgggccagatctgtggcctctcagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggtatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataaggtggaagccgaggtgcagatcgaccggctgattacaggcagactccagagcctccagacctacgtgacacagcagctgatcagagccgccgagattagagcctctgccaatctggccgccatcaagatgagcgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgactcagcggaacttctacgagccccagatcatcaccaccgacaacaccttcgtgtccggcaactgcgacgtcgtgatcggcatcgtgaacaataccgtgtacgaccctctccagccagagctggacagcttcaaagaggaactggacaagtacttcaagaaccacacaagccccgacgtggacctgggcgatattagcggaatcaacgccagcttcgtgaacatccagaaagagatcgacagactgaacgaggtcgccaagaacctgaacgagagcctgatcgacctgcaagagctggggaagtacgagcagtatatcaagtggccctggtacatctggctgggctttatcgccggcctgattgccatcgtgatggtcacaatcatgctgtgctgcatgaccagctgttgcagctgcctgaagggctgctgtagctgcggctcttgttgttaa

SEQ ID NO: 28 - protein, 1265 aa, synthetic construct
  REGION 1..1265: note = SARS-COV2-BR
MVLLLILSVLLLKEDVRGSAQSTGQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCC

SEQ ID NO: 29 - DNA, 3789 bp, synthetic construct
  misc_feature 1..3789: note = SARS-COV2-SA
  source 1..3789: mol_type = other DNA
atggttctgctgctgatcctgtctgtgctgctgctcaaagaagatgtgcggggcagcgcccagtctacaggccagtgtgtgaacttcaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaatcctgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcggggactgcctcagggcttttctgctctggaacccctggtggatctgcccatcggcatcaacatcacccggtttcagaccctgcaccgcagctatctgacacccggcgattctagctctggatggacagctggcgccgctgcctactacgttggctatctccagccaagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaactgcgtggccgattactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagaccggcaatatcgccgactacaactacaagctgcccgacgacttcaccggctgtgtgattgcctggaacagcaacaacctggactccaaagtcggcggcaactacaattacctgtaccggctgtttcggaagtccaatctgaagcccttcgagcgggacatctccaccgagatctatcaggccggcagcaccccttgcaatggcgtgaagggctttaactgctacttcccactgcaaagctacggcttccagccaacatacggcgtgggctaccagccttacagagtggtggtgctgagcttcgagctgctgcatgctcctgccacagtgtgcggccctaagaaaagcaccaatctcgtgaagaacaaatgcgtcaacttcaatttcaacggcctgaccggcaccggcgtgctgacagagagcaacaagaagttcctgccattccagcagttcggccgggacattgccgataccacagatgctgtcagagatccccagacactggaaatcctggacatcaccccatgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgttccagaccagagccggctgtctgatcggagccgagcacgtgaacaatagctacgagtgcgacatccccatcggcgctggaatctgtgctagctaccagacacagacaaacagccccagacgggccagatctgtggcctctcagagcatcattgcctacaccatgagcctgggcgtcgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggtatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgctcaggccctgaacaccctggtcaagcagctgagcagcaacttcggcgccatcagcagcgtgctgaacgacatcctgagccgcctggataaggtggaagccgaggtgcagatcgaccggctgattacaggcagactccagagcctccagacctacgtgacacagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgactcagcggaacttctacgagccccagatcatcaccaccgacaacaccttcgtgtccggcaactgcgacgtcgtgatcggcatcgtgaacaataccgtgtacgaccctctccagccagagctggacagcttcaaagaggaactggacaagtacttcaagaaccacacaagccccgacgtggacctgggcgatattagcggcatcaatgccagcgtcgtgaacatccagaaagagatcgacagactgaacgaggtcgccaagaacctgaacgagagcctgatcgacctgcaagagctggggaagtacgagcagtatatcaagtggccctggtacatctggctgggctttatcgccggcctgattgccatcgtgatggtcacaatcatgctgtgctgcatgaccagctgttgcagctgcctgaagggctgctgtagctgcggctcttgttgttaa

SEQ ID NO: 30 - protein, 1262 aa, synthetic construct
  REGION 1..1262: note = SARS-COV2-SA
MVLLLILSVLLLKEDVRGSAQSTGQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 31 - DNA, 3762 bp, synthetic construct
  misc_feature 1..3762: note = SARS-COV2-OMICRON
  source 1..3762: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatcagcggcaccaatggcaccaagagattcgacaaccccgtgctgcccttcaacgacggggtgtactttgccagcatcgagaagtccaacatcatccgcggctggatcttcggcaccacactggatagcaagacccagagcctgctgatcgtgaacaacgccaccaacgtggtcatcaaagtgtgcgagttccagttctgcaacgacccattcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgcgagcctgaggatctgcctcagggcttttctgccctggaacctctggtggatctgcccatcggcatcaacatcacccggtttcagacactgctggccctgcacagaagctacctgacacctggcgatagcagcagcggatggacagctggtgccgccgcttactacgtgggctatctccagccaagaaccttcctgctgaagtacaacgagaacggcaccatcaccgacgccgtggattgtgctctggatcccctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaatctggccccattcttcaccttcaagtgctacggcgtgtcccctaccaagctgaacgacctgtgcttcaccaatgtgtacgccgacagcttcgtgatccggggagatgaagtgcggcagattgcccctggacagaccggcaatatcgccgactacaactacaagctgcccgacgacttcaccggctgtgtgatcgcctggaatagcaacaagctggacagcaaggtgtccggcaactacaattacctgtaccggctgtttcggaagtccaatctgaagcccttcgagcgggacatcagcaccgagatctatcaggccggcaacaagccctgtaatggcgtggccggcttcaactgctacttcccactgcggagctacagcttcagacccacatacggcgtgggccaccagccttatagagtggtggtgctgagcttcgagctgctgcatgctcctgccacagtgtgcggccctaagaaaagcaccaacctcgtgaagaacaaatgcgtgaacttcaacttcaacggcctgaaaggcaccggcgtgctgaccgagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgttccagaccagagccggctgtctgattggcgccgagtatgtgaacaacagctacgagtgcgacatccccatcggagccggcatctgtgctagctaccagacacagaccaagagccacagacgggccagaagtgtggcctctcagagcatcattgcctacacaatgagcctgggcgtcgagaacagcgtggcctacagcaacaactctatcgctatccccaccaacttcaccatcagcgtgaccaccgagattctgcccgtgtccatgaccaagaccagcgtggactgcaccatgtacatctgcggcgattccaccgagtgctccaacctgctgctccagtacggcagcttctgcacccagctgaagagagccctgacagggattgccgtggaacaggacaagaacacccaagaggtgttcgcccaagtgaagcagatctacaagacccctcctatcaagtacttcggcgggttcaacttctcccagatccttccggaccctagcaagcccagcaagcggagctttatcgaggacctgctgttcaacaaagtgacactggccgacgccggctttatcaagcagtacggcgattgcctgggagatatcgccgccagggatctgatttgcgcccagaagtttaagggcctgacagtgctgcctcctctgctgacagatgagatgatcgcccagtacacaagcgccctgctggccggcacaatcacctctggatggacatttggagccggcgctgccctgcaaatcccctttgctatgcagatggcctatcggttcaacggcatcggagtgacccagaatgtgctgtacgagaaccagaagctgatcgccaaccagttcaacagcgccatcggcaagatccaggacagcctgagcagcacagcctctgctctgggaaagctccaggacgtggtcaaccacaatgctcaggccctgaacaccctggtcaagcagctgtctagcaagttcggcgccatctctagcgtgctgaatgacatcttctccaggctggacaaggtggaagccgaggtgcagatcgacagactgatcaccggtagactccagtctctccagacctacgtgacccagcagctgattagagccgccgagatcagagccagcgccaatctggctgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcaacccgagctggactccttcaaagaggaactggataagtactttaagaaccacacaagccccgacgtggacctgggcgatattagcggcatcaatgccagcgtcgtgaacatccagaaagagatcgaccggctgaacgaggtggcaaagaacctgaacgagagcctgatcgacctgcaagaactggggaagtacgagcagtacatcaagtggccttggtacatctggctgggctttatcgccggactgattgccatcgtgatggtcacaatcatgctgtgctgtatgaccagctgctgtagctgcctgaagggctgttgtagctgtggcagctgttgctctagctag

SEQ ID NO: 32 - protein, 1253 aa, synthetic construct
  REGION 1..1253: note = SARS-COV2-OMICRON
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGVENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCSS

SEQ ID NO: 33 - DNA, 4014 bp, synthetic construct
  misc_feature 1..4014: note = MERS
  source 1..4014: mol_type = other DNA
atgatccactccgtgtttctgctgatgttcctgctgacccctaccgagagctatgtggacgtgggccccgattctgtgaagtccgcctgtatcgaagtggacatccagcagacctttttcgacaagacctggcctcggcctatcgacgtgtccaaggccgacggcatcatctaccctcaaggccggacctacagcaacatcaccattacctaccaggggctgttcccctaccaaggcgatcacggcgatatgtacgtgtactctgccggacacgccaccggcaccacacctcagaaactgttcgtggccaactacagccaggacgtgaagcagttcgccaacggctttgttgtgcggattggcgccgctgccaatagcacaggcacagtgatcatcagccctagcaccagcgccaccatcagaaagatctaccccgccttcatgctgggcagcagcgtgggcaatttcagcgacggcaagatgggcagattcttcaatcacaccctggtgctgctgcctgatggctgtggaacactgctgagagccttctactgcatcctggaacctcggagcggcaatcactgtcctgccggcaatagctacaccagcttcgccacctatcacacccctgccaccgattgctccgacggcaactacaacagaaacgccagcctgaacagcttcaaagagtacttcaacctgcggaactgcaccttcatgtacacctacaatatcaccgaggacgagatcctcgagtggttcggcatcacacagacagctcagggcgtgcacctgttcagcagcagatacgtggacctgtacggcggcaacatgttccagtttgccacactgcccgtgtacgacaccatcaagtactacagcatcatccctcacagcatccggtccatccagagcgacagaaaagcctgggccgccttctacgtgtacaagctccagcctctgaccttcctgctggactttagcgtggacggctacatcagacgggccatcgactgcggcttcaacgatctgtctcagctgcactgctcctacgagagcttcgacgtggaaagcggcgtgtacagcgtgtccagctttgaggccaagcctagcggctctgtggtggaacaagctgaaggcgtggaatgcgacttcagccctctgctgtctggcacacctcctcaggtgtacaacttcaagcggctggtgttcaccaactgcaattacaacctgaccaagctgctgagcctgttctccgtgaacgacttcacctgtagccagatcagccctgccgccattgccagcaactgttacagcagcctgatcctggactacttcagctaccctctgagcatgaagtccgatctgagcgtgtcctccgccggaccaatcagccagttcaactacaagcagtccttcagcaaccctacctgcctgattctggccaccgtgcctcacaatctgaccaccatcaccaagcctctgaagtacagctacatcaacaagtgcagcagactgctgagcgacgacagaaccgaagtgccccagctggtcaacgccaaccagtacagcccttgcgtgtccatcgtgcctagcaccgtttgggaggacggcgactactatcggaagcagctgtctcctctggaaggcggaggatggctggtggcctctggatctacagtggccatgacagagcagctccagatgggctttggcatcaccgtgcagtacggcaccgacaccaatagcgtgtgccctaagctggaattcgccaatgacaccaagatcgccagccagctgggcaactgcgtggaatactctctctacggcgttagcggcagaggcgtgttccagaattgcaccgccgtgggcgtccgccagcagagatttgtgtacgacgcctaccagaacctcgtgggctactacagcgacgacggcaattactactgcctgcgggcctgtgtgtccgtgcctgtgtctgtgatctacgacaaagagacaaagacccacgccacactgttcggcagcgtggcctgtgaacacatcagctccaccatgagccagtactccaggtctacccggtccatgctgaagcggagagacagcacatacggccctctccagacacctgtgggatgtgtgctgggcctcgtgaacagctccctgtttgtggaagattgcaagctgcccctgggccagtctctgtgtgctagccctgacacaccctctacactgacccctagatctgtgcgctctgtgcccggcgaaatgagactggcctctatcgccttcaatcaccccatccaggtggaccagctgaacagcagctacttcaagctgagcatccccaccaacttcagcttcggcgtgacccaagagtacatccagaccaccatccagaaagtgaccgtggactgcaagcagtacgtgtgcaacggcttccagaagtgcgaacagctgctgagagagtacggccagttctgctccaagatcaatcaggccctgcacggcgccaacctgagacaggatgatagcgtgcggaacctgttcgccagcgtgaagtcctctcagagcagccctatcatccctggcttcggcggcgacttcaatctgacactgctggaacccgtgtccatcagcacaggctccagaagcgccagatccgccattgaggacctgctgttcgacaaagtgacaatcgccgatcctggctacatgcagggctacgacgattgcatgcagcagggacctgccagcgccagggatctgatttgtgcccagtatgtggccggctacaaggtgctgcctcctctgatggacgtgaacatggaagccgcctacacctctagcctgctgggatctattgccggcgttggatggacagccggcctgtctagctttgccgccattcctttcgctcagagcatcttctaccggctgaacggcgtgggcatcacccagcaggttctgagcgagaaccagaagctgatcgccaacaagttcaaccaggctctgggcgccatgcagaccggctttaccacaacaaacgaggcctttcagaaagtgcaggacgccgtgaacaacaacgcccaggcactgtctaagctggcctccgagctgagcaataccttcggagccatctctgcctccatcggcgacatcatccagagactggacgtgctggaacaggacgcccagatcgacagactgatcaacggcagactgaccacactgaacgccttcgtggcacagcagctcgtgcgatctgaaagcgctgctctgtctgcccagctggctaaggacaaagtcaacgagtgcgtgaaggcccagagcaagagaagcggcttttgtggacagggcacccacatcgtgtccttcgtggtcaatgcccctaacggcctgtacttcatgcacgtgggctactaccccagcaaccacatcgaagtggtgtccgcctacggcctgtgtgatgccgccaatcctaccaactgtatcgcccctgtgaacggctacttcatcaagaccaacaacacccggatcgtggacgagtggtcttacaccggcagcagcttttacgcccctgagcctatcaccagcctgaacaccaaatacgtggcccctcaagtgacctaccagaacatcagcaccaacctgcctccacctctgctgggcaatagcaccggcatcgacttccaggacgagctggacgagttcttcaagaacgtgtccacatctatccccaacttcggcagcctgacacagatcaacacaaccctgctggacctgacctacgagatgctgagcctgcaacaggtggtcaaggccctgaacgagagctacatcgacctgaaagagctgggcaactacacctactacaacaagtggccctggtacatctggctgggctttatcgctggactggtggcactggccctgtgcgtgttcttcatcctgtgctgtaccggctgcggcaccaactgcatgggcaagctgaaatgcaacagatgctgctga

SEQ ID NO: 34 - protein, 1337 aa, synthetic construct
  REGION 1..1337: note = MERS
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCASPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCC

SEQ ID NO: 35 - DNA, 3711 bp, synthetic construct
  misc_feature 1..3711: note = SARS-COV-1
  source 1..3711: mol_type = other DNA
atgtttatctttctgctgtttctgaccctgaccagcggcagcgacctggatagatgcaccaccttcgacgatgtgcaggcccctaactacacccagcacaccagctctatgcggggcgtgtactaccccgacgagatcttcagaagcgacaccctgtatctgacccaggacctgttcctgcctttctacagcaacgtgaccggcttccacaccatcaaccacaccttcggcaaccctgtgatccccttcaaggacggcatctactttgccgccaccgagaagtccaacgtcgtcagaggatgggtgttcggcagcaccatgaacaacaagagccagagcgtgatcatcatcaacaacagcaccaacgtggtcatccgggcctgcaacttcgagctgtgcgacaacccattcttcgccgtgtccaagcctatgggcacccagacacacaccatgatcttcgacaacgccttcaactgcaccttcgagtacatcagcgacgccttcagcctggacgtgtccgagaaaagcggcaacttcaagcacctgagggaattcgtgttcaagaacaaggatggcttcctgtacgtgtacaagggctaccagcctatcgacgtcgtgcgggatctgcccagcggcttcaataccctgaagcctatcttcaagctgcccctgggcatcaacatcaccaacttcagagccatcctgaccgctttcagccccgctcaggatatttggggcacaagcgccgctgcctacttcgtgggctatctgaagcccaccacctttatgctgaagtacgacgagaacggcaccatcaccgacgccgtggactgtagccaaaatcctctggccgagctgaagtgcagcgtgaagtccttcgagatcgacaagggcatctaccagaccagcaatttcagagtggtgccctccggggatgtcgtgcggttccccaatatcaccaatctgtgccccttcggcgaggtgttcaacgccaccaagtttccctctgtgtacgcctgggagcgcaagaaaatcagcaactgcgtggccgactacagcgtgctgtacaactccaccttcttcagcaccttcaagtgctacggcgtgtccgccacaaagctgaacgacctgtgcttctccaacgtgtacgccgacagcttcgtggtcaaaggcgacgacgttcggcagattgcccctggacaaacaggcgtgatcgccgattacaactacaagctgcctgacgacttcatgggctgcgtgctggcctggaacaccagaaacatcgatgccaccagcaccggcaactacaattacaagtacagatacctgcggcacggcaagctgcggccttttgagagggacatcagcaatgtgccttttagccccgacggcaagccctgcacacctcctgctctgaattgctactggcccctgaacgactacggcttttacaccaccacaggcatcggctatcagccctatagagtggtggtcctgtcctttgagctgctgaatgcccctgccacagtgtgcggacctaagctgagcaccgacctgatcaagaaccagtgcgtgaacttcaacttcaacggcctgaccggcaccggcgtgctgacaccaagcagcaagagattccagcctttccagcagttcggccgggatgtgtccgacttcacagacagcgtcagagatcccaagaccagcgagatcctggacatctccccttgtgcctttggcggagtgtccgtgatcacccctggcacaaatgcctctagcgaagtggccgtgctgtatcaggacgtgaactgcaccgatgtgtccaccgccattcacgccgatcagctgactcccgcttggcggatctatagcacaggcaacaacgtgttccagacacaagccggctgtctgatcggagccgagcatgtggataccagctacgagtgcgacatccctatcggcgctggcatctgtgctagctaccacaccgtgtctctgctgagaagcaccagccagaaatccatcgtggcctacaccatgagcctgggcgccgatagctctatcgcctacagcaacaacacaatcgctatccccaccaacttcagcatcagcatcaccaccgaagtgatgcccgtgtccatggctaagaccagcgtggactgcaacatgtacatctgcggcgacagcaccgagtgcgccaatctgctgctccagtacggcagcttctgcacccagctgaatagagccctgtctggaattgccgccgagcaggacagaaacaccagagaagtgttcgcccaagtgaagcagatgtataagaccccgacactcaagtacttcggcggcttcaacttctcccagatcctgcctgatcctctgaagcccaccaagcggagcttcatcgaggacctgctgttcaacaaagtgaccctggccgacgccggctttatgaagcagtatggcgagtgcctgggcgacatcaacgccagggatctgatctgcgcccagaagttcaacggcctgacagtgctgcctcctctgctgaccgatgatatgatcgccgcctacacagccgctctggtgtctggaacagctaccgccggatggacatttggagctggcgccgctctccagattccattcgctatgcagatggcctaccgcttcaacggcatcggcgtgacccagaacgtgctgtacgagaaccagaagcagatcgccaaccagttcaacaaggccatcagccagatccaagagagcctgaccaccacctctacagccctgggaaagctccaggacgtggtcaaccagaatgctcaggccctgaacaccctggtcaagcagctgagcagcaacttcggcgccatcagcagcgtgctgaacgacatcctgagccggctggataaggtggaagccgaggtgcagatcgaccggctgattacaggcagactccagagcctccagacctacgtgacacagcagctgatcagagccgccgagattagagcctctgccaatctggccgccaccaagatgtctgagtgtgtgctgggccagagcaagagagtggacttttgcggcaagggctaccacctgatgagcttccctcaggctgctcctcacggcgtggtctttctgcacgtgacatacgtgcccagccaagagcggaacttcacaacagcccctgccatctgccacgagggcaaagcctactttcccagagaaggcgtgttcgtgttcaatggcacctcctggtttatcacccagcggaatttcttcagcccgcaaatcatcaccacagacaacaccttcgtgtccggcaactgcgacgtcgtgatcggcatcatcaacaataccgtgtacgaccctctccagcctgagctggacagcttcaaagaggaactggataagtacttcaagaaccacacaagccccgacgtggacctgggcgatattagcggcatcaatgccagcgtcgtgaacatccagaaagagatcgacagactgaacgaggtcgccaagaacctgaacgagtccctgatcgacctgcaagagctggggaagtacgagcagtacatcaagtggccttggtacgtgtggctgggctttatcgccggactgatcgccatcgtgatggtcacaatcctgctgtgctgcatgaccagctgttgcagctgtctgaagggcgcctgtagctgcggctcttgttgttga

SEQ ID NO: 36 - protein, 1236 aa, synthetic construct
  REGION 1..1236: note = SARS-COV-1
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCC

SEQ ID NO: 37 - DNA, 39 bp, synthetic construct
  misc_feature 1..39: note = S1-C TERMINUS
  source 1..39: mol_type = other DNA
agctaccagacacagaccaacagtcccagacgggccaga

SEQ ID NO: 38 - protein, 13 aa, synthetic construct
  REGION 1..13: note = S1-C TERMINUS
SYQTQTNSPRRAR

SEQ ID NO: 39 - protein, 5 aa, synthetic construct
  REGION 1..5: note = PROLINE MOTIF
RLDKV

SEQ ID NO: 40 - DNA, 1653 bp, synthetic construct
  misc_feature 1..1653: note = SARS-COV-2 S2 DOMAIN
  source 1..1653: mol_type = other DNA
tctgtggccagccagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataagcctcctgtggaagccgaggtgcagatcgacagactgatcaccggcagactccagagcctccagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtccggcaactgcgacgtcgtgatcggcatcgtgaacaataccgtgtacgaccctctgcaacccgagctggacagcttcaaagaggaactggacaagtacttcaagaaccacacaagccccgacgtggacctgggcgatattagcggcatcaatgccagcgtcgtgaacatccagaaagagatcgaccggctgaacgaggtggccaagaacctgaacgagagcctgatcgacctgcaagagctggggaagtacgagcagtatatcaagtggccctggtacatctggctgggctttatcgccggcctgattgccatcgtgatggtcacaatcatgctg

SEQ ID NO: 41 - protein, 551 aa, synthetic construct
  REGION 1..551: note = SARS-COV-2 S2 DOMAIN
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIML

SEQ ID NO: 42 - DNA, 2121 bp, synthetic construct
  misc_feature 1..2121: note = MERS S1 DOMAIN
  source 1..2121: mol_type = other DNA
atcgaagtggacatccagcagacctttttcgacaagacctggcctcggcctatcgacgtgtccaaggccgacggcatcatctaccctcaaggccggacctacagcaacatcaccattacctaccaggggctgttcccctaccaaggcgatcacggcgatatgtacgtgtactctgccggacacgccaccggcaccacacctcagaaactgttcgtggccaactacagccaggacgtgaagcagttcgccaacggctttgttgtgcggattggcgccgctgccaatagcacaggcacagtgatcatcagccctagcaccagcgccaccatcagaaagatctaccccgccttcatgctgggcagcagcgtgggcaatttcagcgacggcaagatgggcagattcttcaatcacaccctggtgctgctgcctgatggctgtggaacactgctgagagccttctactgcatcctggaacctcggagcggcaatcactgtcctgccggcaatagctacaccagcttcgccacctatcacacccctgccaccgattgctccgacggcaactacaacagaaacgccagcctgaacagcttcaaagagtacttcaacctgcggaactgcaccttcatgtacacctacaatatcaccgaggacgagatcctcgagtggttcggcatcacacagacagctcagggcgtgcacctgttcagcagcagatacgtggacctgtacggcggcaacatgttccagtttgccacactgcccgtgtacgacaccatcaagtactacagcatcatccctcacagcatccggtccatccagagcgacagaaaagcctgggccgccttctacgtgtacaagctccagcctctgaccttcctgctggactttagcgtggacggctacatcagacgggccatcgactgcggcttcaacgatctgtctcagctgcactgctcctacgagagcttcgacgtggaaagcggcgtgtacagcgtgtccagctttgaggccaagcctagcggctctgtggtggaacaagctgaaggcgtggaatgcgacttcagccctctgctgtctggcacacctcctcaggtgtacaacttcaagcggctggtgttcaccaactgcaattacaacctgaccaagctgctgagcctgttctccgtgaacgacttcacctgtagccagatcagccctgccgccattgccagcaactgttacagcagcctgatcctggactacttcagctaccctctgagcatgaagtccgatctgagcgtgtcctccgccggaccaatcagccagttcaactacaagcagtccttcagcaaccctacctgcctgattctggccaccgtgcctcacaatctgaccaccatcaccaagcctctgaagtacagctacatcaacaagtgcagcagactgctgagcgacgacagaaccgaagtgccccagctggtcaacgccaaccagtacagcccttgcgtgtccatcgtgcctagcaccgtttgggaggacggcgactactatcggaagcagctgtctcctctggaaggcggaggatggctggtggcctctggatctacagtggccatgacagagcagctccagatgggctttggcatcaccgtgcagtacggcaccgacaccaatagcgtgtgccctaagctggaattcgccaatgacaccaagatcgccagccagctgggcaactgcgtggaatactctctctacggcgttagcggcagaggcgtgttccagaattgcaccgccgtgggcgtccgccagcagagatttgtgtacgacgcctaccagaacctcgtgggctactacagcgacgacggcaattactactgcctgcgggcctgtgtgtccgtgcctgtgtctgtgatctacgacaaagagacaaagacccacgccacactgttcggcagcgtggcctgtgaacacatcagctccaccatgagccagtactccaggtctacccggtccatgctgaagcggagagacagcacatacggccctctccagacacctgtgggatgtgtgctgggcctcgtgaacagctccctgtttgtggaagattgcaagctgcccctgggccagtctctgtgtgct

SEQ ID NO: 43 - protein, 707 aa, synthetic construct
  REGION 1..707: note = MERS S1 DOMAIN
IEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCA

SEQ ID NO: 44 - DNA, 1932 bp, synthetic construct
  misc_feature 1..1932: note = SARS-COV-1 S1 DOMAIN
  source 1..1932: mol_type = other DNA
gacctggatagatgcaccaccttcgacgatgtgcaggcccctaactacacccagcacaccagctctatgcggggcgtgtactaccccgacgagatcttcagaagcgacaccctgtatctgacccaggacctgttcctgcctttctacagcaacgtgaccggcttccacaccatcaaccacaccttcggcaaccctgtgatccccttcaaggacggcatctactttgccgccaccgagaagtccaacgtcgtcagaggatgggtgttcggcagcaccatgaacaacaagagccagagcgtgatcatcatcaacaacagcaccaacgtggtcatccgggcctgcaacttcgagctgtgcgacaacccattcttcgccgtgtccaagcctatgggcacccagacacacaccatgatcttcgacaacgccttcaactgcaccttcgagtacatcagcgacgccttcagcctggacgtgtccgagaaaagcggcaacttcaagcacctgagggaattcgtgttcaagaacaaggatggcttcctgtacgtgtacaagggctaccagcctatcgacgtcgtgcgggatctgcccagcggcttcaataccctgaagcctatcttcaagctgcccctgggcatcaacatcaccaacttcagagccatcctgaccgctttcagccccgctcaggatatttggggcacaagcgccgctgcctacttcgtgggctatctgaagcccaccacctttatgctgaagtacgacgagaacggcaccatcaccgacgccgtggactgtagccaaaatcctctggccgagctgaagtgcagcgtgaagtccttcgagatcgacaagggcatctaccagaccagcaatttcagagtggtgccctccggggatgtcgtgcggttccccaatatcaccaatctgtgccccttcggcgaggtgttcaacgccaccaagtttccctctgtgtacgcctgggagcgcaagaaaatcagcaactgcgtggccgactacagcgtgctgtacaactccaccttcttcagcaccttcaagtgctacggcgtgtccgccacaaagctgaacgacctgtgcttctccaacgtgtacgccgacagcttcgtggtcaaaggcgacgacgttcggcagattgcccctggacaaacaggcgtgatcgccgattacaactacaagctgcctgacgacttcatgggctgcgtgctggcctggaacaccagaaacatcgatgccaccagcaccggcaactacaattacaagtacagatacctgcggcacggcaagctgcggccttttgagagggacatcagcaatgtgccttttagccccgacggcaagccctgcacacctcctgctctgaattgctactggcccctgaacgactacggcttttacaccaccacaggcatcggctatcagccctatagagtggtggtcctgtcctttgagctgctgaatgcccctgccacagtgtgcggacctaagctgagcaccgacctgatcaagaaccagtgcgtgaacttcaacttcaacggcctgaccggcaccggcgtgctgacaccaagcagcaagagattccagcctttccagcagttcggccgggatgtgtccgacttcacagacagcgtcagagatcccaagaccagcgagatcctggacatctccccttgtgcctttggcggagtgtccgtgatcacccctggcacaaatgcctctagcgaagtggccgtgctgtatcaggacgtgaactgcaccgatgtgtccaccgccattcacgccgatcagctgactcccgcttggcggatctatagcacaggcaacaacgtgttccagacacaagccggctgtctgatcggagccgagcatgtggataccagctacgagtgcgacatccctatcggcgctggcatctgtgct

SEQ ID NO: 45 - protein, 644 aa, synthetic construct
  REGION 1..644: note = SARS-COV-1 S1 DOMAIN
DLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICA

SEQ ID NO: 46 - DNA, 1971 bp, synthetic construct
  misc_feature 1..1971: note = SARS-COV-2 OMICRON S1 DOMAIN
  source 1..1971: mol_type = other DNA
agccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatcagcggcaccaatggcaccaagagattcgacaaccccgtgctgcccttcaacgacggggtgtactttgccagcatcgagaagtccaacatcatccgcggctggatcttcggcaccacactggatagcaagacccagagcctgctgatcgtgaacaacgccaccaacgtggtcatcaaagtgtgcgagttccagttctgcaacgacccattcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgcgagcctgaggatctgcctcagggcttttctgccctggaacctctggtggatctgcccatcggcatcaacatcacccggtttcagacactgctggccctgcacagaagctacctgacacctggcgatagcagcagcggatggacagctggtgccgccgcttactacgtgggctatctccagccaagaaccttcctgctgaagtacaacgagaacggcaccatcaccgacgccgtggattgtgctctggatcccctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaatctggccccattcttcaccttcaagtgctacggcgtgtcccctaccaagctgaacgacctgtgcttcaccaatgtgtacgccgacagcttcgtgatccggggagatgaagtgcggcagattgcccctggacagaccggcaatatcgccgactacaactacaagctgcccgacgacttcaccggctgtgtgatcgcctggaatagcaacaagctggacagcaaggtgtccggcaactacaattacctgtaccggctgtttcggaagtccaatctgaagcccttcgagcgggacatcagcaccgagatctatcaggccggcaacaagccctgtaatggcgtggccggcttcaactgctacttcccactgcggagctacagcttcagacccacatacggcgtgggccaccagccttatagagtggtggtgctgagcttcgagctgctgcatgctcctgccacagtgtgcggccctaagaaaagcaccaacctcgtgaagaacaaatgcgtgaacttcaacttcaacggcctgaaaggcaccggcgtgctgaccgagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgttccagaccagagccggctgtctgattggcgccgagtatgtgaacaacagctacgagtgcgacatccccatcggagccggcatctgtgct

SEQ ID NO: 47 - protein, 657 aa, synthetic construct
  REGION 1..657: note = SARS-COV-2 OMICRON S1 DOMAIN
SQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICA

SEQ ID NO: 48 - DNA, 3813 bp, synthetic construct
  misc_feature 1..3813: note = MERS/SARS-COV-2 S DOMAIN
  source 1..3813: mol_type = other DNA
atcgaagtggacatccagcagacctttttcgacaagacctggcctcggcctatcgacgtgtccaaggccgacggcatcatctaccctcaaggccggacctacagcaacatcaccattacctaccaggggctgttcccctaccaaggcgatcacggcgatatgtacgtgtactctgccggacacgccaccggcaccacacctcagaaactgttcgtggccaactacagccaggacgtgaagcagttcgccaacggctttgttgtgcggattggcgccgctgccaatagcacaggcacagtgatcatcagccctagcaccagcgccaccatcagaaagatctaccccgccttcatgctgggcagcagcgtgggcaatttcagcgacggcaagatgggcagattcttcaatcacaccctggtgctgctgcctgatggctgtggaacactgctgagagccttctactgcatcctggaacctcggagcggcaatcactgtcctgccggcaatagctacaccagcttcgccacctatcacacccctgccaccgattgctccgacggcaactacaacagaaacgccagcctgaacagcttcaaagagtacttcaacctgcggaactgcaccttcatgtacacctacaatatcaccgaggacgagatcctcgagtggttcggcatcacacagacagctcagggcgtgcacctgttcagcagcagatacgtggacctgtacggcggcaacatgttccagtttgccacactgcccgtgtacgacaccatcaagtactacagcatcatccctcacagcatccggtccatccagagcgacagaaaagcctgggccgccttctacgtgtacaagctccagcctctgaccttcctgctggactttagcgtggacggctacatcagacgggccatcgactgcggcttcaacgatctgtctcagctgcactgctcctacgagagcttcgacgtggaaagcggcgtgtacagcgtgtccagctttgaggccaagcctagcggctctgtggtggaacaagctgaaggcgtggaatgcgacttcagccctctgctgtctggcacacctcctcaggtgtacaacttcaagcggctggtgttcaccaactgcaattacaacctgaccaagctgctgagcctgttctccgtgaacgacttcacctgtagccagatcagccctgccgccattgccagcaactgttacagcagcctgatcctggactacttcagctaccctctgagcatgaagtccgatctgagcgtgtcctccgccggaccaatcagccagttcaactacaagcagtccttcagcaaccctacctgcctgattctggccaccgtgcctcacaatctgaccaccatcaccaagcctctgaagtacagctacatcaacaagtgcagcagactgctgagcgacgacagaaccgaagtgccccagctggtcaacgccaaccagtacagcccttgcgtgtccatcgtgcctagcaccgtttgggaggacggcgactactatcggaagcagctgtctcctctggaaggcggaggatggctggtggcctctggatctacagtggccatgacagagcagctccagatgggctttggcatcaccgtgcagtacggcaccgacaccaatagcgtgtgccctaagctggaattcgccaatgacaccaagatcgccagccagctgggcaactgcgtggaatactctctctacggcgttagcggcagaggcgtgttccagaattgcaccgccgtgggcgtccgccagcagagatttgtgtacgacgcctaccagaacctcgtgggctactacagcgacgacggcaattactactgcctgcgggcctgtgtgtccgtgcctgtgtctgtgatctacgacaaagagacaaagacccacgccacactgttcggcagcgtggcctgtgaacacatcagctccaccatgagccagtactccaggtctacccggtccatgctgaagcggagagacagcacatacggccctctccagacacctgtgggatgtgtgctgggcctcgtgaacagctccctgtttgtggaagattgcaagctgcccctgggccagtctctgtgtgctagctaccagacacagaccaacagtcccagacgggccagatctgtggccagccagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataagcctcctgtggaagccgaggtgcagatcgacagactgatcaccggcagactccagagcctccagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtccggcaactgcgacgtcgtgatcggcatcgtgaacaataccgtgtacgaccctctgcaacccgagctggacagcttcaaagaggaactggacaagtacttcaagaaccacacaagccccgacgtggacctgggcgatattagcggcatcaatgccagcgtcgtgaacatccagaaagagatcgaccggctgaacgaggtggccaagaacctgaacgagagcctgatcgacctgcaagagctggggaagtacgagcagtatatcaagtggccctggtacatctggctgggctttatcgccggcctgattgccatcgtgatggtcacaatcatgctg

SEQ ID NO: 49 - protein, 1271 aa, synthetic construct
  REGION 1..1271: note = MERS/SARS-COV-2 S DOMAIN
IEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIML

SEQ ID NO: 50 - DNA, 3624 bp, synthetic construct
  misc_feature 1..3624: note = SARS-COV-1/SARS-COV-2 S DOMAIN
  source 1..3624: mol_type = other DNA
gacctggatagatgcaccaccttcgacgatgtgcaggcccctaactacacccagcacaccagctctatgcggggcgtgtactaccccgacgagatcttcagaagcgacaccctgtatctgacccaggacctgttcctgcctttctacagcaacgtgaccggcttccacaccatcaaccacaccttcggcaaccctgtgatccccttcaaggacggcatctactttgccgccaccgagaagtccaacgtcgtcagaggatgggtgttcggcagcaccatgaacaacaagagccagagcgtgatcatcatcaacaacagcaccaacgtggtcatccgggcctgcaacttcgagctgtgcgacaacccattcttcgccgtgtccaagcctatgggcacccagacacacaccatgatcttcgacaacgccttcaactgcaccttcgagtacatcagcgacgccttcagcctggacgtgtccgagaaaagcggcaacttcaagcacctgagggaattcgtgttcaagaacaaggatggcttcctgtacgtgtacaagggctaccagcctatcgacgtcgtgcgggatctgcccagcggcttcaataccctgaagcctatcttcaagctgcccctgggcatcaacatcaccaacttcagagccatcctgaccgctttcagccccgctcaggatatttggggcacaagcgccgctgcctacttcgtgggctatctgaagcccaccacctttatgctgaagtacgacgagaacggcaccatcaccgacgccgtggactgtagccaaaatcctctggccgagctgaagtgcagcgtgaagtccttcgagatcgacaagggcatctaccagaccagcaatttcagagtggtgccctccggggatgtcgtgcggttccccaatatcaccaatctgtgccccttcggcgaggtgttcaacgccaccaagtttccctctgtgtacgcctgggagcgcaagaaaatcagcaactgcgtggccgactacagcgtgctgtacaactccaccttcttcagcaccttcaagtgctacggcgtgtccgccacaaagctgaacgacctgtgcttctccaacgtgtacgccgacagcttcgtggtcaaaggcgacgacgttcggcagattgcccctggacaaacaggcgtgatcgccgattacaactacaagctgcctgacgacttcatgggctgcgtgctggcctggaacaccagaaacatcgatgccaccagcaccggcaactacaattacaagtacagatacctgcggcacggcaagctgcggccttttgagagggacatcagcaatgtgccttttagccccgacggcaagccctgcacacctcctgctctgaattgctactggcccctgaacgactacggcttttacaccaccacaggcatcggctatcagccctatagagtggtggtcctgtcctttgagctgctgaatgcccctgccacagtgtgcggacctaagctgagcaccgacctgatcaagaaccagtgcgtgaacttcaacttcaacggcctgaccggcaccggcgtgctgacaccaagcagcaagagattccagcctttccagcagttcggccgggatgtgtccgacttcacagacagcgtcagagatcccaagaccagcgagatcctggacatctccccttgtgcctttggcggagtgtccgtgatcacccctggcacaaatgcctctagcgaagtggccgtgctgtatcaggacgtgaactgcaccgatgtgtccaccgccattcacgccgatcagctgactcccgcttggcggatctatagcacaggcaacaacgtgttccagacacaagccggctgtctgatcggagccgagcatgtggataccagctacgagtgcgacatccctatcggcgctggcatctgtgctagctaccagacacagaccaacagtcccagacgggccagatctgtggccagccagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataagcctcctgtggaagccgaggtgcagatcgacagactgatcaccggcagactccagagcctccagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtccggcaactgcgacgtcgtgatcggcatcgtgaacaataccgtgtacgaccctctgcaacccgagctggacagcttcaaagaggaactggacaagtacttcaagaaccacacaagccccgacgtggacctgggcgatattagcggcatcaatgccagcgtcgtgaacatccagaaagagatcgaccggctgaacgaggtggccaagaacctgaacgagagcctgatcgacctgcaagagctggggaagtacgagcagtatatcaagtggccctggtacatctggctgggctttatcgccggcctgattgccatcgtgatggtcacaatcatgctg

SEQ ID NO: 51 - protein, 1208 aa, synthetic construct
  REGION 1..1208: note = SARS-COV-1/SARS-COV-2 S DOMAIN
DLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIML

SEQ ID NO: 52 - DNA, 3663 bp, synthetic construct
  misc_feature 1..3663: note = SARS-COV-2 OMICRON/SARS-COV-2 ALPHA
  source 1..3663: mol_type = other DNA
agccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatcagcggcaccaatggcaccaagagattcgacaaccccgtgctgcccttcaacgacggggtgtactttgccagcatcgagaagtccaacatcatccgcggctggatcttcggcaccacactggatagcaagacccagagcctgctgatcgtgaacaacgccaccaacgtggtcatcaaagtgtgcgagttccagttctgcaacgacccattcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgcgagcctgaggatctgcctcagggcttttctgccctggaacctctggtggatctgcccatcggcatcaacatcacccggtttcagacactgctggccctgcacagaagctacctgacacctggcgatagcagcagcggatggacagctggtgccgccgcttactacgtgggctatctccagccaagaaccttcctgctgaagtacaacgagaacggcaccatcaccgacgccgtggattgtgctctggatcccctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaatctggccccattcttcaccttcaagtgctacggcgtgtcccctaccaagctgaacgacctgtgcttcaccaatgtgtacgccgacagcttcgtgatccggggagatgaagtgcggcagattgcccctggacagaccggcaatatcgccgactacaactacaagctgcccgacgacttcaccggctgtgtgatcgcctggaatagcaacaagctggacagcaaggtgtccggcaactacaattacctgtaccggctgtttcggaagtccaatctgaagcccttcgagcgggacatcagcaccgagatctatcaggccggcaacaagccctgtaatggcgtggccggcttcaactgctacttcccactgcggagctacagcttcagacccacatacggcgtgggccaccagccttatagagtggtggtgctgagcttcgagctgctgcatgctcctgccacagtgtgcggccctaagaaaagcaccaacctcgtgaagaacaaatgcgtgaacttcaacttcaacggcctgaaaggcaccggcgtgctgaccgagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggcagtgctgtaccagggcgtgaactgtacagaggtgcccgtggctattcacgccgatcagctgacccctacatggcgggtgtactccacaggcagcaatgtgttccagaccagagccggctgtctgattggcgccgagtatgtgaacaacagctacgagtgcgacatccccatcggagccggcatctgtgctagctaccagacacagaccaacagtcccagacgggccagatctgtggccagccagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctccagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctccagattccattcgctatgcagatggcctatcggttcaacggcatcggcgtgacccagaacgtgctgtacgagaaccagaagctgatcgccaaccagttcaacagcgccatcggcaagatccaggacagcctgagcagtacagccagcgctctgggaaagctccaggacgtggtcaaccagaatgcccaggctctgaacaccctggtcaagcagctgagcagcaacttcggcgccatcagcagcgtgctgaacgacatcctgagccgcctggataagcctcctgtggaagccgaggtgcagatcgacagactgatcaccggcagactccagagcctccagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtccggcaactgcgacgtcgtgatcggcatcgtgaacaataccgtgtacgaccctctgcaacccgagctggacagcttcaaagaggaactggacaagtacttcaagaaccacacaagccccgacgtggacctgggcgatattagcggcatcaatgccagcgtcgtgaacatccagaaagagatcgaccggctgaacgaggtggccaagaacctgaacgagagcctgatcgacctgcaagagctggggaagtacgagcagtatatcaagtggccctggtacatctggctgggctttatcgccggcctgattgccatcgtgatggtcacaatcatgctg

SEQ ID NO: 53 - protein, 1221 aa, synthetic construct
  REGION 1..1221: note = SARS-COV-2 OMICRON/SARS-COV-2 ALPHA
SQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIML

SEQ ID NO: 54 - skipped

SEQ ID NO: 55 - protein, 4 aa, synthetic construct
  REGION 1..4: note = Pilot peptide, YxxL motif
YINL

SEQ ID NO: 56 - protein, 4 aa, synthetic construct
  REGION 1..4: note = Pilot peptide, YxxL motif
YSHL

SEQ ID NO: 57 - skipped

SEQ ID NO: 58 - protein, 5 aa, synthetic construct
  REGION 1..5: note = Pilot peptide, DYxxL motif
DYINL

SEQ ID NO: 59-61 - skipped (3 entries)

SEQ ID NO: 62 - protein, 4 aa, synthetic construct
  REGION 1..4: note = Pilot peptide, PxxP motif
PSAP

SEQ ID NO: 63 - protein, 4 aa, synthetic construct
  REGION 1..4: note = Pilot peptide, PxxP motif
PTAP

SEQ ID NO: 64 - protein, 40 aa, synthetic construct
  REGION 1..40: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
PXXPXXXXPXXPXSXYXXLXPXXPEXYXXLXPXXPDYXXL

SEQ ID NO: 65 - protein, 37 aa, synthetic construct
  REGION 1..37: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 14: note = X represents any amino acid residue
  VARIANT 15: note = X represents any amino acid residue
  VARIANT 17: note = X represents any one or several amino acid residue(s)
  VARIANT 19: note = X represents any amino acid residue
  VARIANT 20: note = X represents any amino acid residue
  VARIANT 23: note = X represents any one or several amino acid residue(s)
  VARIANT 25: note = X represents any amino acid residue
  VARIANT 26: note = X represents any amino acid residue
  VARIANT 28: note = X represents any one or several amino acid residue(s)
  VARIANT 30: note = X represents any amino acid residue
  VARIANT 31: note = X represents any amino acid residue
  VARIANT 35: note = X represents any amino acid residue
  VARIANT 36: note = X represents any amino acid residue
PXXPXPXXPXSXYXXLXPXXPEXYXXLXPXXPDYXXL

SEQ ID NO: 66 - protein, 44 aa, synthetic construct
  REGION 1..44: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
  VARIANT 41: note = X represents any amino acid residue
  VARIANT 42: note = X represents any amino acid residue
  VARIANT 43: note = X represents any amino acid residue
  VARIANT 44: note = X represents any amino acid residue
PXXPXXXXPXXPXSXYXXLXPXXPEXYXXLXPXXPDYXXLXXXX

SEQ ID NO: 67 - protein, 41 aa, synthetic construct
  REGION 1..41: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 15: note = X represents any amino acid residue
  VARIANT 17: note = X represents any one or several amino acid residue(s)
  VARIANT 19: note = X represents any amino acid residue
  VARIANT 20: note = X represents any amino acid residue
  VARIANT 23: note = X represents any one or several amino acid residue(s)
  VARIANT 25: note = X represents any amino acid residue
  VARIANT 26: note = X represents any amino acid residue
  VARIANT 28: note = X represents any one or several amino acid residue(s)
  VARIANT 30: note = X represents any amino acid residue
  VARIANT 31: note = X represents any amino acid residue
  VARIANT 35: note = X represents any amino acid residue
  VARIANT 36: note = X represents any amino acid residue
  VARIANT 38: note = X represents any amino acid residue
  VARIANT 39: note = X represents any amino acid residue
  VARIANT 40: note = X represents any amino acid residue
  VARIANT 41: note = X represents any amino acid residue
  VARIANT 14: note = X represents any amino acid residue
PXXPXPXXPXSXYXXLXPXXPEXYXXLXPXXPDYXXLXXXX

SEQ ID NO: 68 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 16: note = X represents any amino acid residue
  VARIANT 18: note = X represents any amino acid residue
  VARIANT 19: note = X represents any amino acid residue
PXXPXXXXXXXXXXXXYXXL

SEQ ID NO: 69 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 18: note = X represents any amino acid residue
  VARIANT 19: note = X represents any amino acid residue
PXXPXXXXXXXXXXXDYXXL

SEQ ID NO: 70 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 16: note = X represents any amino acid residue
  VARIANT 18: note = X represents any amino acid residue
  VARIANT 19: note = X represents any amino acid residue
PXXPXXYXXXXXXXXXYXXL

SEQ ID NO: 71 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 18: note = X represents any amino acid residue
  VARIANT 19: note = X represents any amino acid residue
PXXPXXYXXXXXXXXDYXXL

SEQ ID NO: 72 - protein, 20 aa, synthetic construct
  REGION 1..20: note = Pilot peptide
  VARIANT 2: note = X represents any amino acid residue
  VARIANT 3: note = X represents any amino acid residue
  VARIANT 6: note = X represents any amino acid residue
  VARIANT 8: note = X represents any amino acid residue
  VARIANT 9: note = X represents any amino acid residue
  VARIANT 11: note = X represents any amino acid residue
  VARIANT 13: note = X represents any amino acid residue
  VARIANT 14: note = X represents any amino acid residue
  VARIANT 18: note = X represents any amino acid residue
  VARIANT 19: note = X represents any amino acid residue
PXXPEXYXXLXPXXPDYXXL

SEQ ID NO: 73 - protein, 9 aa, synthetic construct
  REGION 1..9: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
PXXPXYXXL

SEQ ID NO: 74 - protein, 10 aa, synthetic construct
  REGION 1..10: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 8: note = X represents any amino acid residue
  VARIANT 9: note = X represents any amino acid residue
PXXPXDYXXL

SEQ ID NO: 75 - protein, 11 aa, synthetic construct
  REGION 1..11: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any one or several amino acid residue(s)
  VARIANT 9: note = X represents any amino acid residue
  VARIANT 10: note = X represents any amino acid residue
PXXPXYXYXXL

SEQ ID NO: 76 - protein, 12 aa, synthetic construct
  REGION 1..12: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any one or several amino acid residue(s)
  VARIANT 10: note = X represents any amino acid residue
  VARIANT 11: note = X represents any amino acid residue
PXXPXYXDYXXL

SEQ ID NO: 77 - protein, 20 aa, synthetic construct
  REGION 1..20: note = Pilot peptide
  VARIANT 2: note = X represents any amino acid residue
  VARIANT 3: note = X represents any amino acid residue
  VARIANT 6: note = X represents any one or several amino acid residue(s)
  VARIANT 8: note = X represents any amino acid residue
  VARIANT 9: note = X represents any amino acid residue
  VARIANT 11: note = X represents any one or several amino acid residue(s)
  VARIANT 13: note = X represents any amino acid residue
  VARIANT 14: note = X represents any amino acid residue
  VARIANT 18: note = X represents any amino acid residue
  VARIANT 19: note = X represents any amino acid residue
PXXPEXYXXLXPXXPDYXXL

SEQ ID NO: 78 - protein, 40 aa, synthetic construct
  REGION 1..40: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 14: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
PXXPXXXXPXXPXXXYXXLXPXXPEXYXXLXPXXPDYXXL

SEQ ID NO: 79 - protein, 35 aa, synthetic construct
  REGION 1..35: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 13: note = X represents any amino acid residue
  VARIANT 15: note = X represents any one or several amino acid residue(s)
  VARIANT 17: note = X represents any amino acid residue
  VARIANT 18: note = X represents any amino acid residue
  VARIANT 21: note = X represents any one or several amino acid residue(s)
  VARIANT 23: note = X represents any amino acid residue
  VARIANT 24: note = X represents any amino acid residue
  VARIANT 26: note = X represents any one or several amino acid residue(s)
  VARIANT 28: note = X represents any amino acid residue
  VARIANT 29: note = X represents any amino acid residue
  VARIANT 33: note = X represents any amino acid residue
  VARIANT 34: note = X represents any amino acid residue
PXXPXPXXPXYXXLXPXXPEXYXXLXPXXPDYXXL

SEQ ID NO: 80 - protein, 44 aa, synthetic construct
  REGION 1..44: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 14: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
  VARIANT 41: note = X represents any amino acid residue
  VARIANT 42: note = X represents any amino acid residue
  VARIANT 43: note = X represents any amino acid residue
  VARIANT 44: note = X represents any amino acid residue
PXXPXXXXPXXPXXXYXXLXPXXPEXYXXLXPXXPDYXXLXXXX

SEQ ID NO: 81 - protein, 39 aa, synthetic construct
  REGION 1..39: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several mino acid residue(s)
  VARIANT 12: note = X represents any amino acid residue
  VARIANT 13: note = X represents any amino acid residue
  VARIANT 15: note = X represents any one or several amino acid residue(s)
  VARIANT 17: note = X represents any amino acid residue
  VARIANT 18: note = X represents any amino acid residue
  VARIANT 21: note = X represents any one or several amino acid residue(s)
  VARIANT 23: note = X represents any amino acid residue
  VARIANT 24: note = X represents any amino acid residue
  VARIANT 26: note = X represents any one or several amino acid residue(s)
  VARIANT 28: note = X represents any amino acid residue
  VARIANT 29: note = X represents any amino acid residue
  VARIANT 33: note = X represents any amino acid residue
  VARIANT 34: note = X represents any amino acid residue
  VARIANT 36: note = X represents any amino acid residue
  VARIANT 37: note = X represents any amino acid residue
  VARIANT 38: note = X represents any amino acid residue
  VARIANT 39: note = X represents any amino acid residue
PXXPXPXXPXYXXLXPXXPEXYXXLXPXXPDYXXLXXXX

SEQ ID NO: 82 - protein, 14 aa, synthetic construct
  REGION 1..14: note = SIGNAL PEPTIDE SARS-COV-1 STRAIN TOR2
MFIFLLFLTLTSGS

SEQ ID NO: 83 - DNA, 42 bp, synthetic construct
  misc_feature 1..42: note = SIGNAL PEPTIDE SARS-COV-1 STRAIN TOR2
  source 1..42: mol_type = other DNA
atgtttatctttctgctgtttctgaccctgaccagcggcagc

SEQ ID NO: 84 - protein, 30 aa, synthetic construct
  REGION 1..30: note = SIGNAL PEPTIDE MERS
MIHSVFLLMFLLTPTESYVDVGPDSVKSAC

SEQ ID NO: 85 - DNA, 90 bp, synthetic construct
  misc_feature 1..90: note = SIGNAL PEPTIDE MERS
  source 1..90: mol_type = other DNA
atgatccactccgtgtttctgctgatgttcctgctgacccctaccgagagctatgtggacgtgggccccgattctgtgaagtccgcctgt

SEQ ID NO: 86 - protein, 12 aa, synthetic construct
  REGION 1..12: note = SIGNAL PEPTIDE SARS-COV-2 OMICRON
MFVFLVLLPLVS

SEQ ID NO: 87 - DNA, 36 bp, synthetic construct
  misc_feature 1..36: note = SIGNAL PEPTIDE SARS-COV-2 OMICRON
  source 1..36: mol_type = other DNA
atgttcgtgtttctggtgctgctgcctctggtgtcc

SEQ ID NO: 88 - protein, 37 aa, synthetic construct
  REGION 1..37: note = CYTOSOLIC DOMAIN
MTSCCSCLKGCCSCGSCCKFDEDDSEPVLKGVKLHYT

SEQ ID NO: 89 - protein, 23 aa, synthetic construct
  REGION 1..23: note = TRANSMEMBRANE DOMAIN
WYIWLGFIAGLIAIVMVTIMLCC

SEQ ID NO: 90 - protein, 19 aa, synthetic construct
  REGION 1..19: note = CYTOSOLIC DOMAIN
KFDEDDSEPVLKGVKLHYT